(19) United States
(12) Patent Application Publication
(10) Pub. No.: US 2010/0144760 A1
(43) Pub. Date: Jun. 10, 2010

(54) NOVEL COMPOUNDS

(76) Inventors: Giuseppe ALVARO, Verona (IT); David Amantini, Verona (IT); Emiliano Castiglioni, Verona (IT); Romano Di Fabio, Verona (IT); Francesca Pavone, Verona (IT)

Correspondence Address: GlaxoSmithKline;GLOBAL PATENTS -US, UW2220, P. O. BOX 1539, KING OF PRUSSIA, PA, 19406-0939, US

(21) Appl. No.: 12/627,134

(22) Filed: Nov. 30, 2009

Related U.S. Application Data
(60) Provisional application No. 61/119,126, filed on Dec. 2, 2008.
(60) Provisional application No. 61/185,316, filed on Jun. 9, 2009.

(51) Int. Cl. A61K 31/506; C07D 401/14; A61P 25/20; A61P 25/24; A61P 25/22; A61P 25/30; A61P 11/00; A61P 9/00

(52) U.S. Cl. 514/256; 544/333

(57) ABSTRACT

Disclosed are N-{[(1R,4S,6R)-3-(2-pyridinylcarbonyl)-3-azabicyclo[4.1.0]hept-4-yl]methyl}-2-heteroarylamine derivatives and their use as pharmaceuticals.

This application claims the benefit of U.S. Provisional Application Nos. 61/185,316, filed 9 Jun. 2009, and 61/119,126, filed 2 Dec. 2008.

### BACKGROUND OF THE INVENTION

This invention relates to N-{[(1R,4S,6R)-3-(2-pyridinylcarbonyl)-3-azabicyclo[4.1.0]hept-4-yl]methyl}-2-heteroarylamine derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP875565, EP875566 and WO 96/34877. Polypeptides and polynucleotides encoding a second human orexin receptor, orexin-2 (HFGANP), have been identified and are disclosed in EP893498.

Polypeptides and polynucleotides encoding polypeptides which are ligands for the orexin-1 receptor, e.g. orexin-A (Lig72A) are disclosed in EP849361.

The orexin ligand and receptor system has been well characterised since its discovery (see for example Sakurai, T. et al (1998) Cell, 92 pp 573 to 585; Smart et al (1999) British Journal of Pharmacology 128 pp 1 to 3; Willie et al (2001) Ann. Rev. Neurosciences 24 pp 429 to 458; Sakurai (2007) Nature Reviews Neuroscience 8 pp 171 to 181; Ohno and Sakurai (2008) Front. Neuroendocrinology 29 pp 70 to 87). From these studies it has become clear that orexins and orexin receptors play a number of important physiological roles in mammals and open up the possibility of the development of new therapeutic treatments for a variety of diseases and disorders as described hereinbelow.

Experiments have shown that central administration of the ligand orexin-A stimulated food intake in freely-feeding rats during a 4 hour time period. This increase was approximately four-fold over control rats receiving vehicle. These data suggest that orexin-A may be an endogenous regulator of appetite (Sakurai, T. et al (1998) Cell, 92 pp 573 to 585; Peyron et al (1998) J. Neurosciences 18 pp 9996 to 10015; Willie et al (2001) Ann. Rev. Neurosciences 24 pp 429 to 458). Therefore, antagonists of the orexin-A receptor(s) may be useful in the treatment of obesity and diabetes. In support of this it has been shown that orexin receptor antagonist SB334867 potently reduced hedonic eating in rats (White et al (2005) Peptides 26 pp 2231 to 2238) and also attenuated high-fat pellet self-administration in rats (Nair et al (2008) British Journal of Pharmacology, published online 28 Jan. 2008).

The search for new therapies to treat obesity and other eating disorders is an important challenge. According to WHO definitions a mean of 35% of subjects in 39 studies were overweight and a further 22% clinically obese in westernised societies. It has been estimated that 5.7% of all healthcare costs in the USA are a consequence of obesity. About 85% of Type 2 diabetics are obese. Diet and exercise are of value in all diabetics. The incidence of diagnosed diabetes in westernised countries is typically 5% and there are estimated to be an equal number undiagnosed. The incidence of obesity and Type 2 diabetes is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

As well as having a role in food intake, the orexin system is also involved in sleep and wakefulness. Rat sleep/EEG studies have shown that central administration of orexin-A, an agonist of the orexin receptors, causes a dose-related increase in arousal, largely at the expense of a reduction in paradoxical sleep and slow wave sleep 2, when administered at the onset of the normal sleep period (Hagan et al (1999) Proc.Natl.Acad.Sci. 96 pp 10911 to 10916). The role of the orexin system in sleep and wakefulness is now well established (Sakurai (2007) Nature Reviews Neuroscience 8 pp 171 to 181; Ohno and Sakurai (2008) Front. Neuroendocrinology 29 pp 70 to 87; Chemelli et al (1999) Cell 98 pp 437 to 451; Lee et al (2005) J. Neuroscience 25 pp 6716 to 6720; Piper et al (2000) European J Neuroscience 12 pp 726-730 and Smart and Jerman (2002) Pharmacology and Therapeutics 94 pp 51 to 61). Antagonists of the orexin receptors may therefore be useful in the treatment of sleep disorders including insomnia. Studies with orexin receptor antagonists, for example SB334867, in rats (see for example Smith et al (2003) Neuroscience Letters 341 pp 256 to 258) and more recently dogs and humans (Brisbare-Roch et al (2007) Nature Medicine 13(2) pp 150 to 155) further support this.

In addition, recent studies have suggested a role for orexin antagonists in the treatment of motivational disorders, such as disorders related to reward seeking behaviours for example drug addiction and substance abuse (Borgland et al (2006) Neuron 49(4) pp 589-601; Boutrel et al (2005) Proc.Natl.Acad.Sci. 102(52) pp 19168 to 19173; Harris et al (2005) Nature 437 pp 556 to 559).

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO05/118548 discloses substituted 1,2,3,4-tetrahydroisoquinoline derivatives as orexin antagonists.

WO01/96302, WO02/44172, WO02/89800, WO03/002559, WO03/002561, WO03/032991, WO03/037847, WO03/041711, WO08/038,251, WO09/003,993, WO09/003,997 and WO09/124,956 all disclose cyclic amine derivatives.

WO08/038,251 discloses 3-aza-bicyclo[3.1.0]hexane derivatives as orexin antagonists.

### SUMMARY OF THE INVENTION

We have now found that N-{[(1R,4S,6R)-3-(2-pyridinylcarbonyl)-3-azabicyclo[4.1.0]hept-4-yl]methyl}-2-heteroarylamine derivatives have beneficial properties including, for example, high potency, good brain penetration and good bioavailability. Such properties make these N-{[(1R,4S,6R)-3-(2-pyridinylcarbonyl)-3-azabicyclo[4.1.0]hept-4-yl]methyl}-2-heteroarylamine derivatives very attractive as potential pharmaceutical agents which may be useful in the prevention or treatment of obesity, including obesity observed in Type 2 (non-insulin-dependent) diabetes patients, sleep disorders, anxiety, depression, schizophrenia, drug dependency or compulsive behaviour. Additionally these compounds may be useful in the treatment of stroke, particularly ischemic or haemorrhagic stroke, and/or blocking the emetic response, i.e. useful in the treatment of nausea and vomiting.

Accordingly the present invention provides a compound of formula (I)

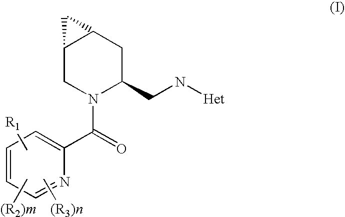

wherein:
Het is a heteroaryl group selected from the group consisting of pyridinyl, pyrimidinyl, pyridazinyl or pyrazinyl, said heteroaryl group being optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano;
R₁ is C₁₋₄alkyl, halo, C₁₋₄alkoxy, haloC₁₋₄alkyl, haloC₁₋₄alkoxy, cyano, C₁₋₄alkylSO₂, C₃₋₈ cycloalkylSO₂, C₃₋₈cycloalkylCH₂SO₂, phenyl or a 5 or 6 membered heterocyclyl group containing 1, 2 or 3 atoms selected from N, O or S, which phenyl or heterocyclyl group is optionally substituted with C₁₋₄alkyl, halo, C₁₋₄alkoxy, haloC₁₋₄alkyl, haloC₁₋₄alkoxy or cyano;
R₂ is C₁₋₄alkyl, halo, C₁₋₄alkoxy, haloC₁₋₄alkyl, haloC₁₋₄alkoxy, cyano, phenyl or a 5 or 6 membered heterocyclyl group containing 1, 2 or 3 atoms selected from N, O or S, which phenyl or heterocyclyl group is optionally substituted with C₁₋₄alkyl, halo, C₁₋₄alkoxy, haloC₁₋₄alkyl, haloC₁₋₄alkoxy or cyano;
R₃ is C₁₋₄alkyl, halo, C₁₋₄alkoxy, haloC₁₋₄alkyl, haloC₁₋₄alkoxy or cyano;
m is 0 or 1; and
n is 0 or 1;
or a pharmaceutically acceptable salt thereof.

### DETAILED DESCRIPTION OF THE INVENTION

In one embodiment Het is a heteroaryl group selected from the group consisting of pyridinyl, pyrimidinyl, pyridazinyl or pyrazinyl, said heteroaryl group being optionally substituted with 1, 2 or 3 substituents independently selected from the group consisting of: C₁₋₄alkyl, halo, C₁₋₄alkoxy, haloC₁₋₄alkyl, haloC₁₋₄alkoxy and cyano;

R₁ is C₁₋₄alkyl, halo, C₁₋₄alkoxy, haloC₁₋₄alkyl, haloC₁₋₄alkoxy, cyano, C₁₋₄alkylSO₂, C₃₋₈ cycloalkylSO₂, C₃₋₈cycloalkylCH₂SO₂, phenyl or a 5 or 6 membered heterocyclyl group containing 1, 2 or 3 atoms selected from N, O or S, which phenyl or heterocyclyl group is optionally substituted with 1 or 2 groups selected from C₁₋₄alkyl, halo, C₁₋₄alkoxy, haloC₁₋₄alkyl, haloC₁₋₄alkoxy or cyano;
R₂ is C₁₋₄alkyl, halo, C₁₋₄alkoxy, haloC₁₋₄alkyl, haloC₁₋₄alkoxy, cyano, phenyl or a 5 or 6 membered heterocyclyl group containing 1, 2 or 3 atoms selected from N, O or S, which phenyl or heterocyclyl group is optionally substituted with 1 or 2 groups selected from C₁₋₄alkyl, halo, C₁₋₄alkoxy, haloC₁₋₄alkyl, haloC₁₋₄alkoxy or cyano;
R₃ is C₁₋₄alkyl, halo, C₁₋₄alkoxy, haloC₁₋₄alkyl, haloC₁₋₄alkoxy or cyano;
m is 0 or 1; and
n is 0 or 1;
or a pharmaceutically acceptable salt thereof.

In one embodiment Het is substituted with haloC₁₋₄alkyl.

In another embodiment Het is substituted with trifluoromethyl.

In one embodiment Het is pyridinyl.

In one embodiment Het is pyridazinyl.

In one embodiment Het is pyrazinyl.

In one embodiment Het is pyrimidinyl.

In another embodiment Het is pyridinyl substituted with trifluoromethyl or cyano.

In another embodiment Het is pyrimidinyl substituted with 1 or 2 CH₃ groups.

In one embodiment m and n are both 0.

In one embodiment m is 1 and n is 0.

In one embodiment R₁ is CH₃.

In another embodiment R₁ is CH₃ and m and n are both 0.

In one embodiment R₂ is methoxy, ethoxy or propoxy.

In another embodiment R₂ is phenyl, pyrimidinyl, oxadiazolyl, oxazolyl, isoxazolyl, thiazolyl, triazolyl, imidazolyl, pyrazolinyl, pyridazinyl, pyrazinyl or pyridinyl.

In a further embodiment R₂ is phenyl substituted with fluoro.

In a still further embodiment R₂ is oxadiazolyl, oxazolyl or thiazolyl substituted with methyl.

In a still further embodiment R₂ is oxadiazolyl, oxazolyl or thiazolyl substituted with ethyl.

In one embodiment m is 1, n is 0, R₁ is CH₃ and R₂ is methoxy, ethoxy or propoxy.

In one embodiment Het is pyridinyl, m is 1, n is 0, R₁ is CH₃, R₂ is methoxy, ethoxy or propoxy.

In another embodiment Het is pyridinyl substituted with trifluoromethyl or cyano, m is 1, n is 0, R₁ is CH₃ and R₂ is methoxy, ethoxy or propoxy.

In one embodiment Het is pyrimidinyl, m is 1, n is 0, R₁ is CH₃ and R₂ is methoxy, ethoxy or propoxy.

In another embodiment Het is pyrimidinyl substituted with 1 or 2 CH₃ groups, m is 1, n is 0, R₁ is CH₃ and R₂ is methoxy, ethoxy or propoxy.

In one embodiment Het is pyridinyl substituted with trifluoromethyl, m is 1, n is 0, R₁ is CH₃ and R₂ is pyrimidinyl.

In one embodiment Het is pyrazinyl substituted with trifluoromethyl, m is 1, n is 0, R₁ is CH₃ and R₂ is pyrimidinyl, or a pharmaceutically acceptable salt thereof.

In one embodiment the invention provides the compound of formula (I) selected from the group consisting of:
N-[((1R,4S,6R)-3-{[6-methyl-3-(propyloxy)-2-pyridinyl]carbonyl}-3-azabicyclo[4.1.0]hept-4-yl)methyl]-5-(trifluoromethyl)-2-pyridinamine;
N-({(1R,4S,6R)-3-[(6-methyl-2-pyridinyl)carbonyl]-3-azabicyclo[4.1.0]hept-4-yl}methyl)-5-(trifluoromethyl)-2-pyridinamine;
N-[((1R,4S,6R)-3-{[6-methyl-3-(methyloxy)-2-pyridinyl]carbonyl}-3-azabicyclo[4.1.0]hept-4-yl)methyl]-5-(trifluoromethyl)-2-pyridinamine;
N-[((1R,4S,6R)-3-{[3-(ethyloxy)-6-methyl-2-pyridinyl]carbonyl}-3-azabicyclo[4.1.0]hept-4-yl)methyl]-5-(trifluoromethyl)-2-pyridinamine;
N-[((1R,4S,6R)-3-{[3-(4-fluorophenyl)-6-methyl-2-pyridinyl]carbonyl}-3-azabicyclo[4.1.0]hept-4-yl)methyl]-5-(trifluoromethyl)-2-pyridinamine;
N-({(1R,4S,6R)-3-[(6-methyl-3-phenyl-2-pyridinyl)carbonyl]-3-azabicyclo[4.1.0]hept-4-yl}methyl)-5-(trifluoromethyl)-2-pyridinamine;
N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(3-methyl-1,2,4-oxadiazol-5-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[3-(5-ethyl-1,3-oxazol-2-yl)-6-methyl-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(4-methyl-1,3-thiazol-2-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(2H-1,2,3-triazol-2-yl)-2-pyridinyl]carbonyl}-3-azabicyclo[4.1.0]hept-4-yl)methyl]-5-(trifluoromethyl)-2-pyridinamine;
6-{[((1R,4S,6R)-3-{[6-methyl-3-(propyloxy)-2-pyridinyl]carbonyl}-3-azabicyclo[4.1.0]hept-4-yl)methyl]amino}-3-pyridinecarbonitrile;
N-[((1R,4S,6R)-3-{[3-(ethyloxy)-6-methyl-2-pyridinyl]carbonyl}-3-azabicyclo[4.1.0]hept-4-yl)methyl]-4,6-dimethyl-2-pyrimidinamine,
N-[((1R,4S,6R)-3-{[6-methyl-3-(3-methyl-1H-pyrazol-1-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(1H-pyrazol-1-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[3-(4,5-dimethyl-2H-1,2,3-triazol-2-yl)-6-methyl-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(4-methyl-2H-1,2,3-triazol-2-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(2-methyl-4-pyrimidinyl)-2-pyridinyl]carbonyl}-3-azabicyclo[4.1.0]hept-4-yl)methyl]-5-(trifluoromethyl)-2-pyridinamine;
N-({(1R,4S,6R)-3-[(6,6′-dimethyl-2,3′-bipyridin-2′-yl)carbonyl]-3-azabicyclo[4.1.0]hept-4-yl}methyl)-5-(trifluoromethyl)-2-pyridinamine;
N-[((1R,4S,6R)-3-{[6-methyl-3-(3-methyl-1H-1,2,4-triazol-1-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[3-(5-fluoro-2-pyrimidinyl)-6-methyl-2-pyridinyl]carbonyl}-3-azabicyclo[4.1.0]hept-4-yl)methyl]-5-(trifluoromethyl)-2-pyridinamine;
N-{[(1R,4S,6R)-3-({6-methyl-3-[5-(trifluoromethyl)-2-pyrimidinyl]-2-pyridinyl}carbonyl)-3-azabicyclo[4.1.0]hept-4-yl]methyl}-5-(trifluoromethyl)-2-pyridinamine;
N-[((1R,4S,6R)-3-{[6-methyl-3-(3-pyridazinyl)-2-pyridinyl]carbonyl}-3-azabicyclo[4.1.0]hept-4-yl)methyl]-5-(trifluoromethyl)-2-pyridinamine;
N-({(1R,4S,6R)-3-[(6′-methyl-2,3′-bipyridin-2′-yl)carbonyl]-3-azabicyclo[4.1.0]hept-4-yl}methyl)-5-(trifluoromethyl)-2-pyridinamine;
N-[((1R,4S,6R)-3-{[6-methyl-3-(2-pyrazinyl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(5-methyl-2-pyryridinyl]carbonyl}-3-azabicyclo[4.1imidinyl)-2-p.0]hept-4-yl)methyl]-5-(trifluoromethyl)-2-pyridinamine;
N-[((1R,4S,6R)-3-{[3-(4,6-dimethyl-2-pyrimidinyl)-6-methyl-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(4-methyl-2-pyrimidinyl)-2-pyridinyl]carbonyl}-3-azabicyclo[4.1.0]hept-4-yl)methyl]-5-(trifluoromethyl)-2-pyridinamine;
N-({(1R,4S,6R)-3-[(6-methyl-3,3′-bipyridin-2-yl)carbonyl]-3-azabicyclo[4.1.0]hept-4-yl}methyl)-5-(trifluoromethyl)-2-pyridinamine;
N-[((1R,4S,6R)-3-{[6-methyl-3-(1H-1,2,4-triazol-1-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-4-(2-pyrimidinyl)-2-pyridinyl]carbonyl}-3-azabicyclo[4.1.0]hept-4-yl)methyl]-5-(trifluoromethyl)-2-pyridinamine;
N-[((1R,4S,6R)-3-{[3-(2-pyrimidinyl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(2-pyrimidinyl)-2-pyridinyl]carbonyl}-3-azabicyclo[4.1.0]hept-4-yl)methyl]-6-(trifluoromethyl)-3-pyridazinamine;
N-[((1R,4S,6R)-3-{[6-methyl-3-(2-pyrimidinyl)-2-pyridinyl]carbonyl}-3-azabicyclo[4.1.0]hept-4-yl)methyl]-6-(trifluoromethyl)-3-pyrimidinamine;
N-[((1R,4S,6R)-3-{[6-methyl-3-(4-methyl-1H-imidazol-1-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(5-methyl-1,3-oxazol-2-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[3-(4-fluoro-1H-imidazol-1-yl)-6-methyl-2-pyridinyl]carbonyl}-3-azabicyclo[4.1.0]hept-4-yl)methyl]-5-(trifluoromethyl)-2-pyridinamine;
N-{[(1R,4S,6R)-3-({6-methyl-3-[4-(trifluoromethyl)-1H-imidazol-1-yl]-2-pyridinyl}carbonyl)-3-azabicyclo[4.1.0]hept-4-yl]methyl}-5-(trifluoromethyl)-2-pyridinamine;
N-[((1R,4S,6R)-3-{[6-methyl-3-(1,3-thiazol-2-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[3-(4,5-dimethyl-1,3-oxazol-2-yl)-6-methyl-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(3-methyl-5-isoxazolyl)-2-pyridinyl]carbonyl}-3-azabicyclo[4.1.0]hept-4-yl)methyl]-5-(trifluoromethyl)-2-pyridinamine;
N-{[(1R,4S,6R)-3-({6-methyl-3-[(1-methylethyl)oxy]-2-pyridinyl}carbonyl)-3-azabicyclo[4.1.0]hept-4-yl]methyl}-5-(trifluoromethyl)-2-pyridinamine;
6-{[((1R,4S,6R)-3-{[6-methyl-3-(2-pyrimidinyl)-2-pyridinyl]carbonyl}-3-azabicyclo[4.1.0]hept-4-yl)methyl]amino}-4-(trifluoromethyl)-3-pyridinecarbonitrile;
3-fluoro-N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyrazinamine;
N-[((1R,4S,6R)-3-{[3-methyl-6-(2-pyrimidinyl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(5-methyl-1,3-oxazol-2-yl)-2-pyridinyl]carbonyl}-3-azabicyclo[4.1.0]hept-4-yl)methyl]-5-(trifluoromethyl)-2-pyrimidinamine;
N-[((1R,4S,6R)-3-{[6-methyl-3-(5-methyl-1,3,4-oxadiazol-2-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[6-methyl-3-(2-pyrimidinyl)-2-pyridinyl]carbonyl}-3-azabicyclo[4.1.0]hept-4-yl)methyl]-6-(trifluoromethyl)-2-pyrazinamine;
N-({(1R,4S,6R)-3-[(3,6′-dimethyl-2,3′-bipyridin-2′-yl)carbonyl]-3-azabicyclo[4.1.0]hept-4-yl}methyl)-5-(trifluoromethyl)-2-pyridinamine;
N-[((1R,4S,6R)-3-{[6-methyl-3-(4-methyl-1H-pyrazol-1-yl)-2-pyridinyl]carbonyl}-3-azabicyclo[4.1.0]hept-4-yl)methyl]-5-(trifluoromethyl)-2-pyridinamine;
N-[((1R,4S,6R)-3-{[5-methyl-6-(2-pyrimidinyl)-2-pyridinyl]carbonyl}-3-azabicyclo[4.1.0]hept-4-yl)methyl]-5-(trifluoromethyl)-2-pyridinamine;
N-{[(1R,4S,6R)-3-({3-[(cyclopropylmethyl)oxy]-6-methyl-2-pyridinyl}carbonyl)-3-azabicyclo[4.1.0]hept-4-yl]methyl}-5-methyl-2-pyridinamine;
N-[((1R,4S,6R)-3-{[3-(ethyloxy)-6-methyl-2-pyridinyl]carbonyl}-3-azabicyclo[4.1.0]hept-4-yl)methyl]-5-methyl-2-pyrimidinamine;
N-[((1R,4S,6R)-3-{[3-(ethyloxy)-6-methyl-2-pyridinyl]carbonyl}-3-azabicyclo[4.1.0]hept-4-yl)methyl]-5-(trifluoromethyl)-2-pyrimidinamine;
N-[((1R,4S,6R)-3-{[6-methyl-3-(propyloxy)-2-pyridinyl]carbonyl}-3-azabicyclo[4.1.0]hept-4-yl)methyl]-5-(trifluoromethyl)-2-pyrimidinamine;
5,6-dimethyl-N-[((1R,4S,6R)-3-{[6-methyl-3-(propyloxy)-2-pyridinyl]carbonyl}-3-azabicyclo[4.1.0]hept-4-yl)methyl]-2-pyrazinamine; and
N-[((1R,4S,6R)-3-{[6-methyl-3-(4-methyl-1,3-oxazol-2-yl)-2-pyridinyl]carbonyl}-3-azabicyclo[4.1.0]hept-4-yl)methyl]-5-(trifluoromethyl)-2-pyrimidinamine.
   or a pharmaceutically acceptable salt thereof.

The Het group (pyridinyl, pyrimidinyl, pyridazinyl or pyrazinyl) may be attached to the aminomethyl linker by means of a bond between the nitrogen atom in said linker and any carbon or suitable nitrogen atom in said pyridinyl, pyrimidinyl, pyridazinyl or pyrazinyl ring. Preferably the Het group is attached to the linker by means of a bond between the nitrogen atom in the linker and a carbon atom in the Het group ring.

When R₁ or R₂ is a heterocyclic group it can be any 5 or 6 membered heterocyclyl group containing 1, 2 or 3 atoms selected from N, O or S. Examples of such heterocyclic groups include pyrimidinyl, oxadiazolyl, oxazolyl, isoxazolyl, thiazolyl, triazolyl, imidazolyl, pyrazolinyl, pyridazinyl, pyrazinyl or pyridinyl.

When R₁ or R₂ is a heterocyclic group, said group may be attached to the pyridyl ring by means of a bond between a carbon atom of said pyridyl ring and a carbon or a suitable heteroatom of the heterocyclic group. For example where R₂ is a triazolyl group the attachment to the pyridyl ring may be by means of a bond between a carbon atom on the pyridyl ring and a) one of the two carbon atoms orb) one of the three nitrogen atoms of the triazolyl group.

When the compound contains a C₁₋₄alkyl group, whether alone or forming part of a larger group, e.g. C₁₋₄alkoxy, the alkyl group may be straight chain, branched or cyclic, or combinations thereof. Examples of C₁₋₄alkyl are methyl or ethyl.

Examples of haloC₁₋₄alkyl include trifluoromethyl (i.e. —CF₃).

Examples of C₁₋₄alkoxy include methoxy and ethoxy.

Examples of haloC₁₋₄alkoxy include trifluoromethoxy (i.e. —OCF₃).

Halogen or “halo” (when used, for example, in haloC₁₋₄alkyl) means fluoro, chloro, bromo or iodo.

It is to be understood that the present invention covers all combinations of particularised groups and substituents described herein above.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse J. Pharm. Sci (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and represent another aspect of this invention.

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

The stereogenic centres of the compounds of formula (I) are in a trans (1R,4S,6R)-configuration. The invention also extends to any tautomeric forms or mixtures thereof.

The subject invention also includes isotopically-labeled compounds which are identical to those recited in formula (I) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

### Schemes

According to a further aspect of the invention there is provided a process for the preparation of compounds of formula (I) or salts thereof. The following schemes are examples of synthetic schemes that may be used to synthesise the compounds of the invention.

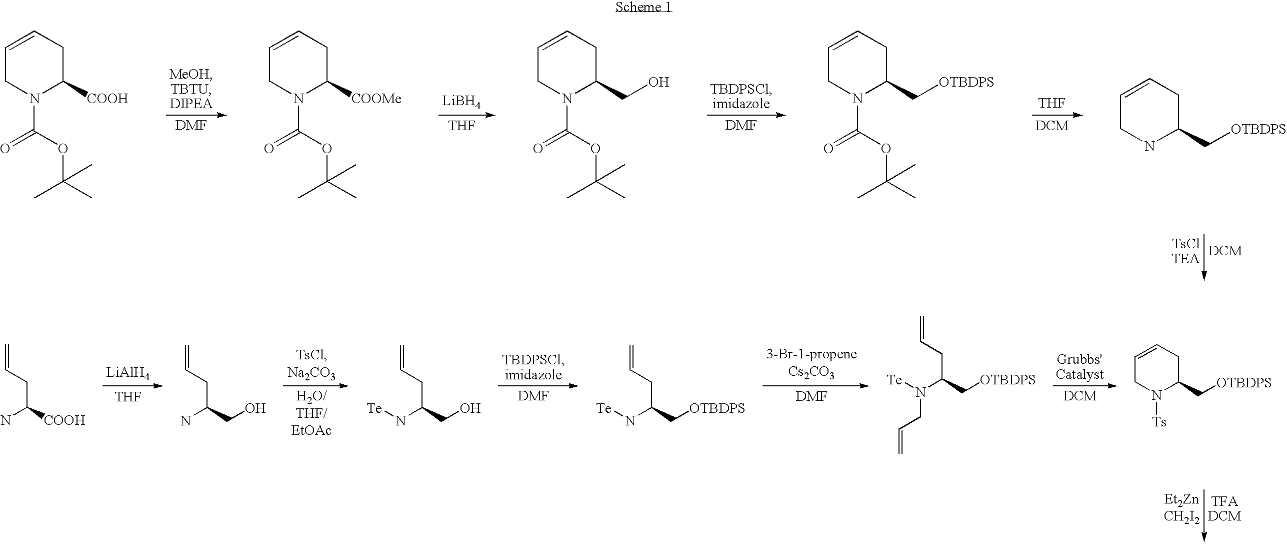

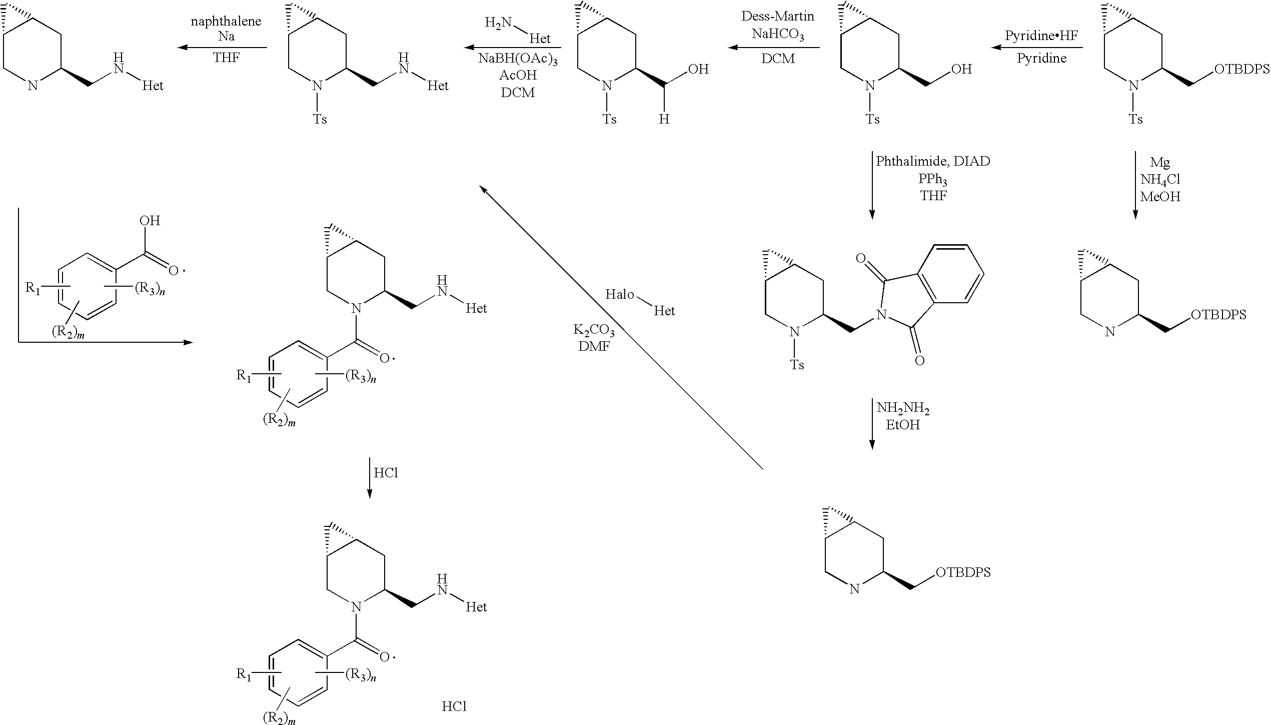

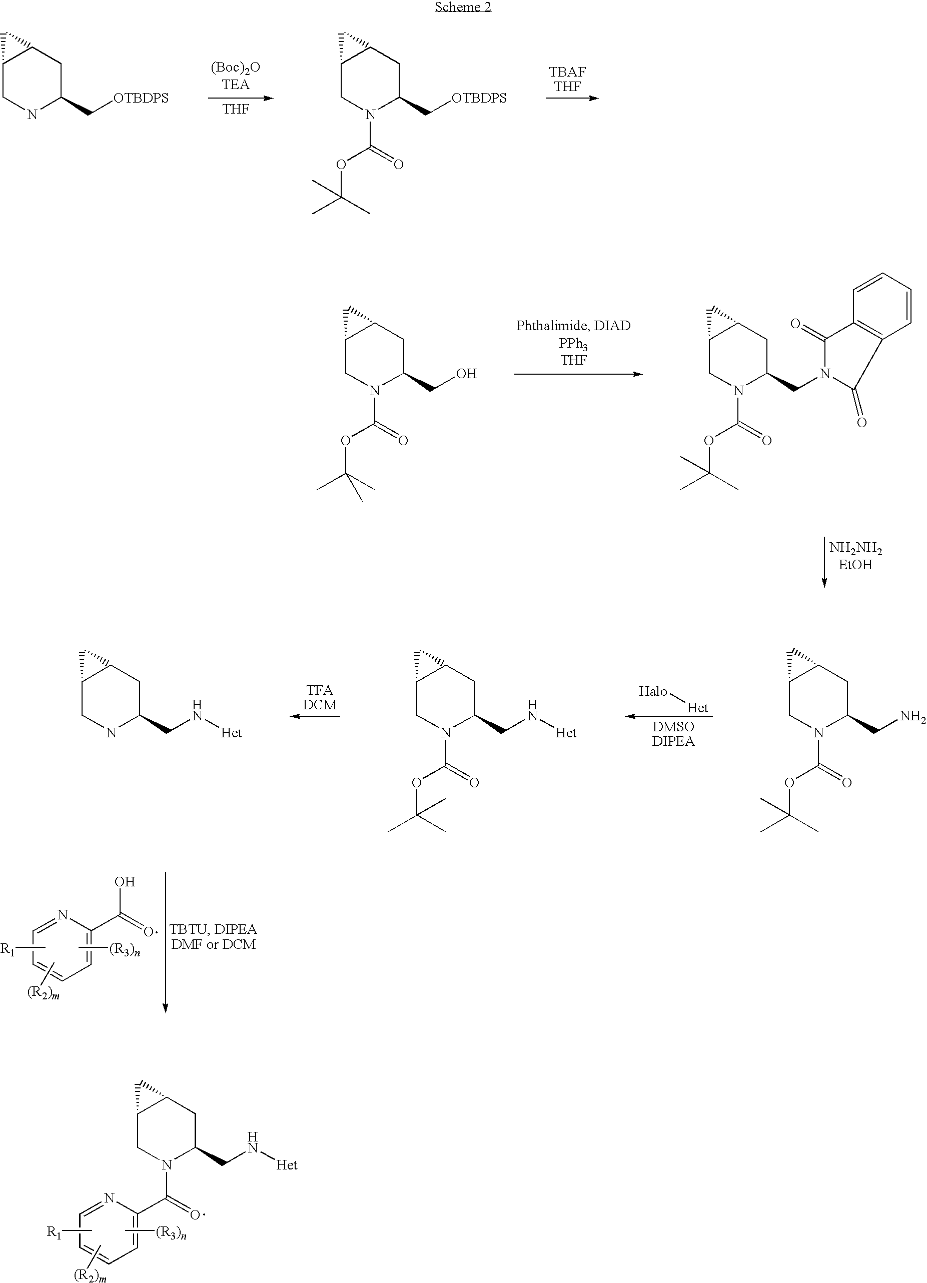

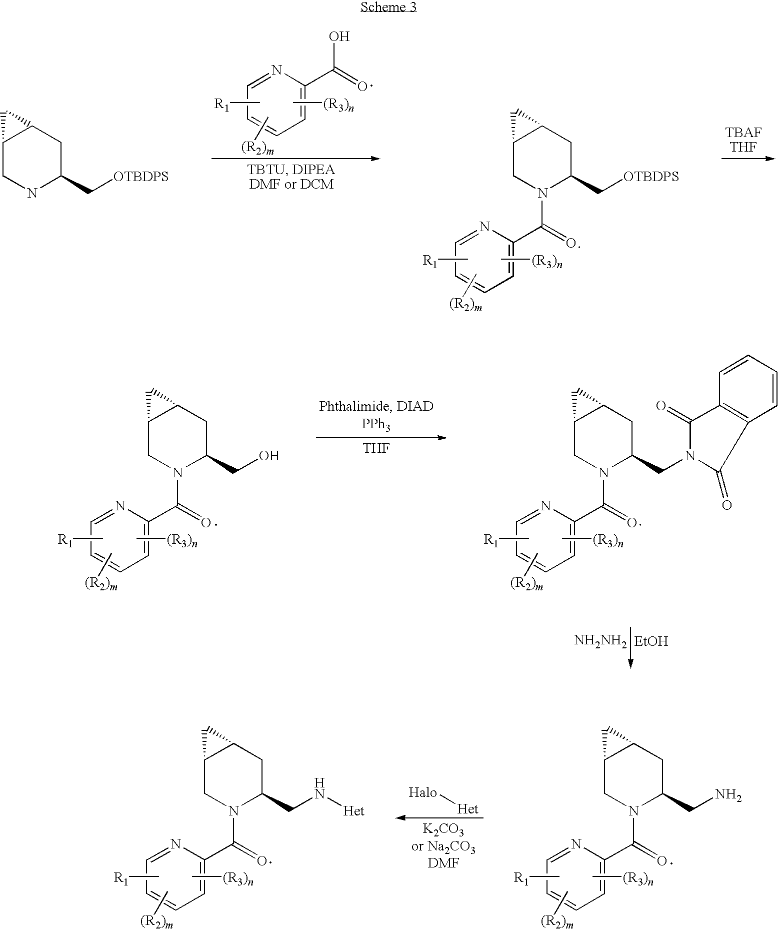

In the schemes Het, R₁, R₂, R₃, m and n have the meanings given in formula (I).

It will be understood by those skilled in the art that certain compounds of the invention can be converted into other compounds of the invention according to standard chemical methods.

The starting materials for use in the scheme are commercially available, known in the literature or can be prepared by known methods. Both (2S)-2-amino-4-pentanoic acid and 1-(1,1-dimethylethyl) 2-methyl (2S)-3,6-dihydro-1,2(2H)-pyridinedicarboxylate are available from Aldrich (Product Number 285013 and 670286 respectively).

Pharmaceutically acceptable salts may be prepared conventionally by reaction with the appropriate acid or acid derivative.

The present invention provides compounds of formula (I) or a pharmaceutically acceptable salt thereof for use in human or veterinary medicine.

The compounds of formula (I) or their pharmaceutically acceptable salts may be of use for the treatment or prophylaxis of a disease or disorder where an antagonist of a human orexin receptor is required.

Compounds of formula (I) or their pharmaceutically acceptable salts may be of use for the treatment or prophylaxis of sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In one embodiment compounds of formula (I) or their pharmaceutically acceptable salts may be of use for the treatment or prophylaxis of Primary Insomnia (307.42), Circadian Rhythm Sleep Disorder (307.45) and Dyssomnia Not Otherwise Specified (307.47), Sleep Disorders Related to Another Mental Disorder such as Insomnia Related to Another Mental Disorder (307.42) and Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type.

In addition the compounds of formula (I) or their pharmaceutically acceptable salts may be of use for the treatment or prophylaxis of depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the compounds of formula (I) or their pharmaceutically acceptable salts may be of use for the treatment or prophylaxis of anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the compounds of formula (I) or their pharmaceutically acceptable salts may be of use for the treatment or prophylaxis of substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the compounds of formula (I) or their pharmaceutically acceptable salts may be of use for the treatment or prophylaxis of feeding disorders such as bulimia nervosa, binge eating, obesity, including obesity observed in Type 2 (non-insulin-dependent) diabetes patients. Further, the compounds of formula (I) or their pharmaceutically acceptable salts may be of use for the treatment or prophylaxis of stroke, particularly ischemic or haemorrhagic stroke and/or in blocking an emetic response i.e. nausea and vomiting.

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

The invention also provides a method for the treatment of a disease or disorder in a subject, for example those diseases and disorders mentioned hereinabove, comprising administering to said subject an effective amount of a compound of formula (I), or a pharmaceutically acceptable salt thereof.

The invention also provides a compound of formula (I), or a pharmaceutically acceptable salt thereof, for use in the treatment or prophylaxis of a disease or disorder, for example those diseases and disorders mentioned hereinabove.

The invention also provides the use of a compound of formula (I), or a pharmaceutically acceptable salt thereof, in the manufacture of a medicament for use in the treatment or prophylaxis of a disease or disorder, for example those diseases and disorders mentioned hereinabove.

For use in therapy the compounds of the invention are usually administered as a pharmaceutical composition. The invention also provides a pharmaceutical composition comprising a compound of formula (I), or a pharmaceutically acceptable salt thereof, and a pharmaceutically acceptable carrier.

The compounds of formula (I) or their pharmaceutically acceptable salts may be administered by any convenient method, e.g. by oral, parenteral, buccal, sublingual, nasal, rectal or transdermal administration, and the pharmaceutical compositions adapted accordingly.

The compounds of formula (I) or their pharmaceutically acceptable salts which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment the composition is in unit dose form such as a tablet, capsule or ampoule.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, and such unit doses may be administered more than once a day, for example two or three a day. Such therapy may extend for a number of weeks or months.

Orexin-A (Sakurai, T. et al (1998) Cell, 92 pp 573-585) can be employed in screening procedures for compounds which inhibit the ligand's activation of the orexin-1 or orexin-2 receptors.

In general, such screening procedures involve providing appropriate cells which express the orexin-1 or orexin-2 receptor on their surface. Such cells include cells from mammals, yeast, Drosophila or E. coli. In particular, a polynucleotide encoding the orexin-1 or orexin-2 receptor is used to transfect cells to express the receptor. The expressed receptor is then contacted with a test compound and an orexin-1 or orexin-2 receptor ligand, as appropriate, to observe inhibition of a functional response. One such screening procedure involves the use of melanophores which are transfected to express the orexin-1 or orexin-2 receptor, as described in WO 92/01810.

Another screening procedure involves introducing RNA encoding the orexin-1 or orexin-2 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 or orexin-2 receptor ligand to cells which have the orexin-1 or orexin-2 receptor (as appropriate) on their surface. This method involves transfecting a eukaryotic cell with DNA encoding the orexin-1 or orexin-2 receptor such that the cell expresses the receptor on its surface and contacting the cell or cell membrane preparation with a compound in the presence of a labelled form of an orexin-1 or orexin-2 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 or orexin-2 receptor ligand with the orexin-1 or orexin-2 receptor as appropriate.

Throughout the specification and claims which follow, unless the context requires otherwise, the word ‘comprise’, and variations such as ‘comprises’ and ‘comprising’ will be understood to imply the inclusion of a stated integer or step or group of integers or steps but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of certain compounds of formula (I) or salts thereof. The Descriptions 1 to 138 illustrate the preparation of intermediates used to make compounds of formula (I) or salts thereof.

In the procedures that follow, after each starting material, reference to a description is typically provided. This is provided merely for assistance to the skilled chemist. The starting material may not necessarily have been prepared from the Description referred to.

The yields were calculated assuming that products were 100% pure if not stated otherwise.

The compounds described in the Examples described hereinafter have all been prepared as a first step from stereo chemically pure starting materials. The stereochemistry of the compounds of the Descriptions and Examples have been assigned on the assumption that the absolute configuration of these centres are retained. The relative stereochemistry of the compounds of the Descriptions and Examples have been assigned on the assumption that the relative stereochemistry is maintained as determined by using Rotating frame 2D ROESY experiments in the chiral intermediates {(1R,4S,6R)-3-[(4-methylphenyl)sulfonyl]-3-azabicyclo[4.1.0]hept-4-yl}methanol D10, N-[(1R,4S,6R)-3-azabicyclo[4.1.0]hept-4-ylmethyl]-5-(trifluoromethyl)-2-pyridinamine D14, [((1R,4S,6R)-3-{[6-methyl-3-(2-pyrimidinyl)-2-pyridinyl]carbonyl}-3-azabicyclo[4.1.0]hept-4-yl)methyl]amine D25. In some Examples the relative stereochemistry has also been experimentally confirmed.

Compounds are named using ACD/Name PRO6.02 chemical naming software (Advanced Chemistry Development Inc., Toronto, Ontario, M5H2L3, Canada).

Proton Magnetic Resonance (NMR) spectra were recorded either on Varian instruments at 400, 500 or 600 MHz, or on a Bruker instrument at 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is usually reported.

Unless otherwise specified, UPLC analyses indicated by UPLC (walk-up): rt (retention time)=x min, were performed on a Agilent 1100 series instrument using a Luna 3u C18(2) 100A column (50×2.0 mm, 3 μm particle size) [Mobile phase and Gradient: 100% (water+0.05% TFA) to 95% (acetonitrile+0.05% TFA) in 8 min. Column T=40° C. Flow rate=1 mL/min. UV detection wavelength=220 nm]. Other UPLC analyses, indicated by UPLC (walk-up, 3 min method), were performed using an Agilent Zorbax SB-C18 column (50×3.0 mm, 1.8 μm particle size) [Mobile phase and Gradient: (Solvent A: water+0.05% TFA) (Solvent B: acetonitrile+0.05% TFA) Gradient: time 0 min 0% B. From 0 to 95% B in 2.5 min. 95% B for 0.2 min. From 95 to 100% B in 0.2 min. 100% B for 0.4 min. From 100% to 0% B in 0.1 min. Flow rate=1.5 mL/min. UV detection wavelength=220 nm]

In the analytical characterization of the described compounds “MS” refers to Mass Spectra taken by Direct infusion Mass or to Mass Spectra associated with peaks taken by UPLC/MS or UPLC/MS analysis, where the Mass Spectrometer used is as mentioned below.

Direct infusion Mass spectra (MS) were run on a Agilent MSD 1100 Mass Spectrometer, operating in ES (+) and ES (−) ionization mode [ES (+): Mass range: 100-1000 amu. Infusion solvent: water+0.1% HCO₂H/CH₃CN 50/50. ES (−): Mass range: 100-1000 amu. Infusion solvent: water+0.05% NH₄OH/CH₃CN 50/50]

MS spectra associated with the peaks were taken on UPLC instrument Perkin Elmer 200 series coupled to an Applied Biosystems API150EX Mass Spectrometer.
UV and MS spectra associated with the peaks were taken on UPLC instrument Agilent 1100 Series coupled to an Agilent LC/MSD 1100 Mass Spectrometer operating in positive or negative electrospray ionization mode and in both acidic and basic gradient conditions [Acidic gradient LC/MS−ES (+ or −): analyses performed on a Supelcosil ABZ+Plus column (33×4.6 mm, 3 μm). Mobile phase: A—water+0.1% HCO₂H/B—CH₃CN. Gradient (standard method): t=0 min 0% (B), from 0% (B) to 95% (B) in 5 min lasting for 1.5 min, from 95% (B) to 0% (B) in 0.1 min, stop time 8.5 min. Column T=room temperature. Flow rate=1 mL/min. Gradient (fast method): t=0 min 0% (B), from 0% (B) to 95% (B) in 3 min lasting for 1 min, from 95% (B) to 0% (B) in 0.1 min, stop time 4.5 min. Column T=room temperature. Flow rate=2 mL/min.

Basic gradient LC/MS−ES (+ or −): analyses performed on a XTerra MS C18 column (30×4.6 mm, 2.5 μm). Mobile phase: A—5 mM aq. NH₄HCO₃+ammonia (pH 10)/B—CH₃CN. Gradient: t=0 min 0% (B), from 0% (B) to 50% (B) in 0.4 min, from 50% (B) to 95% (B) in 3.6 min lasting for 1 min, from 95% (B) to 0% (B) in 0.1 min, stop time 5.8 min. column temperature=room temperature. Flow rate=1.5 mL/min].

Mass range ES (+ or −): 100-1000 amu. UV detection range: 220-350 nm. The usage of this methodology is indicated by “LC-MS” in the analytic characterization of the described compounds.

Total ion current (TIC) and DAD UV chromatographic traces together with MS and UV spectra associated with the peaks were taken on a UPLC/MS Acquity™ system equipped with 2996 PDA detector and coupled to a Waters Micromass ZQ™ Mass Spectrometer operating in positive or negative electrospray ionisation mode [LC/MS−ES (+ or −): analyses performed using an Acquity™ UPLC BEH C18 column (50×21 mm, 1.7 μm particle size), column temperature 40° C.]. Mobile phase: A—water+0.1% HCOOH/B—CH₃CN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B). The usage of this methodology is indicated by “UPLC” in the analytic characterization of the described compounds.

[LC/MS−ES (+ or −): analyses performed using an Acquity™ UPLC BEH C18 column (50×2.1 mm, 1.7 μm particle size) column temperature 40° C.]. Mobile phase: A—water+0.1% HCO₂H/B—CH₃CN+0.06% or 0.1% HCO₂H. Gradient: t=0 min 3% B, t=1.5 min 100% B, t=1.9 min 100% B, t=2 min 3% B stop time 2 min. Column T=40° C. Flow rate=1.0 mL/min. Mass range: ES (+): 100-1000 amu or ES(+): 50-800 amu. ES (−): 100-800 amu. UV detection range: 210-350 nm. The usage of this methodology is indicated by “UPLC (Acid IPQC)” in the analytic characterization of the described compounds.
[LC/MS−ES (+ or −): analyses performed using an Acquity™ UPLC BEH C18 column (50×2.1 mm, 1.7 μm particle size) column temperature 40° C.]. Mobile phase: A—water+0.1% HCO₂H/B—CH₃CN+0.06% or 0.1% HCO₂H. Gradient: t=0 min 3% B, t=0.05 min 6% B, t=0.57 min 70% B, t=1.06 min 99% B lasting for 0.389 min, t=1.45 min 3% B, stop time 1.5 min. Column T=40° C. Flow rate=1.0 mL/min. Mass range: ES (+): 100-1000 amu or ES(+): 50-800 amu, ES (−): 100-800 amu. UV detection range: 210-350 nm. The usage of this methodology is indicated by “UPLC (Acid QC_POS_{—}50-800 or QC_POS_{—}70_{—}900 or GEN_QC or FINAL_QC)” in the analytic characterization of the described compounds. [LC/MS−ES (+ or −): analyses performed using an Acquity™ UPLC BEH C18 column (50×2.1 mm, 1.7 μm particle size) column temperature 40° C.]. Mobile phase: A—water+0.1% HCO₂H/B—CH₃CN+0.06% or 0.1% HCO₂H. Gradient: t=0 min 3% B, t=1.06 min 99% B, t=1.45 min 99% B, t=1.46 min 3% B, stop time 1.5 min. Column T=40° C. Flow rate=1.0 mL/min. Mass range: ES (+): 100-1000 amu. ES (−): 100-800 amu. UV detection range: 210-350 nm. The usage of this methodology is indicated by “UPLC (Acid GEN_QC_SS)” in the analytic characterization of the described compounds.

Total ion current (TIC) and DAD UV chromatographic traces together with MS and UV spectra associated with the peaks were taken on a UPLC/MS Acquity™ system equipped with PDA detector and coupled to a Waters SQD mass spectrometer operating in positive and negative alternate electrospray ionisation mode [LC/MS−ES(+ or −): analyses performed using an Acquity™ UPLC BEH C18 column (50×2.1 mm, 1.7 μm particle size) column temperature 40° C.]. Mobile phase: A—10 mM aqueous solution of NH₄HCO₃ (adjusted to pH 10 with ammonia)/B—CH₃CN. Gradient: t=0 min 3% B, t=1.06 min 99% B lasting for 0.39 min, t=1.46 min 3% B, stop time 1.5 min. Column T=40° C. Flow rate=1.0 mL/min. Mass range: ES (+): 100-1000 amu or ES (+): 50-800 amu. ES (−): 100-1000 amu. UV detection range: 220-350 nm. The usage of this methodology is indicated by “UPLC (Basic GEN_QC or QC_POS_{—}50-800)” in the analytic characterization of the described compounds.

Unless otherwise specified, Preparative LC-MS purifications were run on a MDAP (Mass Detector Auto Purification) Waters instrument (MDAP FractionLynx). [LC/MS−ES (+): analyses performed using a Gemini C18 AXIA column (50×21 mm, 5 μm particle size). Mobile phase: A—NH₄HCO₃ sol. 10 mM, pH 10; B—CH₃CN. Flow rate: 17 ml/min]. The gradient will be specified each time:

[AA_Prep_Purification: gradient: t=0 min 20% B, t=8 min 50% B, t=10 min 100% B, t=11 min 20% B]

[CUSTOM_Prep_Purification: gradient: t=0 min 1% B, t=99 min 30% B, t=9.5 min 100% B, t=10.5 min 1% B].

Preparative LC-MS purifications were also run on a MDAP (Mass Detector Auto Purification) Waters instrument. The usage of this methodology is indicated by “Fraction Lynx” in the analytic characterization of the described compounds. Sunfire Prep. C18 OBD (150 mm×30 mm i.d. 5 μm particle size) at room temperature. The injection volume was: 990 μl. Mobile phase: A=0.1% v/v solution of HCO₂H in water. B=0.1% v/v solution of HCO₂H in CH₃CN. Flow rate: 40 ml/min. [Method Acid LC1 gradient: t=0 min 1% B, t=10 min 25% B, t=14.5 min 90% B, t=15 min 90% B, stop time 15 min.]

For reactions involving microwave irradiation, a Personal Chemistry Emrys™ Optimizer was used.

In a number of preparations, purification was performed using Biotage manual flash chromatography (Flash+), Biotage automatic flash chromatography (Horizon, SP1 and SP4), Companion CombiFlash (ISCO) automatic flash chromatography, Flash Master Personal or Vac Master systems.

Flash chromatography was carried out on silica gel 230-400 mesh (supplied by Merck AG Darmstadt, Germany), Varian Mega Be—Si pre-packed cartridges, pre-packed Biotage silica cartridges (e.g. Biotage SNAP cartridge), KP-NH prepacked flash cartridges or ISCO RediSep Silica cartridges.

SPE-SCX cartridges are ion exchange solid phase extraction columns supplied by Varian. The eluent used with SPE-SCX cartridges is DCM and MeOH or ACN or MeOH followed by 2 N ammonia solution in MeOH. The collected fractions are those eluted with the ammonia solution in MeOH.

SPE-Si cartridges are silica solid phase extraction columns supplied by Varian.

ENV+ cartridges are packed with ENV+ a hyper cross-linked hydroxylated polystyrene-divinylbenzene copolymer.

The following table lists the used abbreviations:

### ACN Acetonitrile

AcOH Acetic acid
bs or br.s. broad signal
Boc t-Butoxycarbonyl
Burgess reagent Methyl N-(triethylammoniumsulphonyl)carbamate
CV Column volumes

### Cy Cyclohexanes

### DCE Dichloroethane

### DCM Dichloromethane

Dess-Martin 1,1,1-Tris(acetoxy)-1,1-dihydro-1,2-benziodoxol-3-(1H)-one periodinane
DIAD Diisopropyl azodicarboxylate

### DIPEA N,N-Diisopropyl-N-ethylamine

### DMF Dimethylformamide

### DMSO Dimethylsulfoxide

### EtOAc Ethylacetate

### EtOH Ethanol

### MeOH Methanol

### min Minutes

MTBE Methyl tertiary butyl ether
NMP N-Methyl-2-pyrrolidone

### Ph Phenyl

pH=3 buffer Citric acid/NaOH/HCl in water solution available from Merck solution KGaA
Grubbs 1^{st} Benzylidene-bis(tricyclohexylphosphine)dichlororuthenium generation

### (Grubbs I)

rt retention time
T temperature
TBAF Tetrabutylammonium fluoride
TBDMS tert-Butyl dimethylsilyl
TBDPS tert-Butyl diphenylsilyl
TBTU O-(benzotriazol-1-yl)-N,N,N′N′-tetramethyluronium tetrafluoroborate

### TEA Triethylamine

TEMPO 2,2,6,6-Tetramethylpiperidine-1-oxyl
TFA Trifluoroacetic acid

### THF Tetrahydrofuran

### TMS Trimethylsilyl

Ts p-Toluensulfonyl

### DESCRIPTIONS

### Description 1

### 1-(1,1-dimethylethyl) 2-methyl (2S)-3,6-dihydro-1,2(2H)-pyridinedicarboxylate (D1)

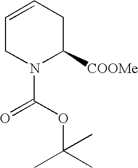

To a solution of (2S)-1-{[(1,1-dimethylethyl)oxy]carbonyl}-1,2,3,6-tetrahydro-2-pyridinecarboxylic acid (1.50 g, 6.60 mmol) in DMF (6 ml), DIPEA (6.92 ml, 39.60 mmol) and TBTU (2.97 g, 9.24 mmol) were added and the mixture stirred at room temperature for 45 min. MeOH (1.42 ml, 35.10 mmol) was added and the resulting reaction mixture stirred for 2 hours. The mixture was diluted with DCM and washed with a saturated NaHCO₃ aqueous solution. The organic layer was separated, dried (Na₂SO₄), filtered through a phase separator tube and concentrated under reduced pressure. The crude material was purified by flash chromatography on silica gel (Flash Master 70 g, Cy/EtOAc 90/10). Collected fractions gave the title compound D1 (1.10 g). MS: (ES/+) m/z: 242 (M+1), 186 [M+1−C(Me)₃)] and 142 (M+1−Boc). C₁₂H₁₉NO₄ requires 241. ¹H-NMR (400 MHz, CDCl₃) δ (ppm): 5.60-5.82 (m, 2H), 4.84-5.15 (m, 1H), 4.01-4.19 (m, 1H), 3.75-3.89 (m, 1H), 3.69-3.76 (m, 3H), 2.44-2.72 (m, 2H), 1.45-1.55 (m, 9H).

### Description 2

### 1,1-dimethylethyl (2S)-2-(hydroxymethyl)-3,6-dihydro-1(2H)-pyridinecarboxylate (D2)

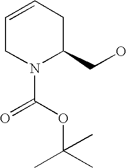

A solution of 1-(1,1-dimethylethyl) 2-methyl (2S)-3,6-dihydro-1,2(2H)-pyridinedicarboxylate D1 (1.10 g) in THF (25 ml) was cooled down to 0° C. and lithium borohydride (2.3 M solution in THF, 4.96 ml, 11.40 mmol) was added dropwise. The resulting reaction mixture was stirred at room temperature overnight. Further lithium borohydride (9.92 ml, 22.80 ml) was added, the mixture was stirred for 6 hours and then quenched with brine and extracted with EtOAc. The organic phase was separated, dried (Na₂SO₄), filtered through a phase separator tube and concentrated under reduced pressure to afford the title compound D2 (0.98 g). The material was used in the next step without any further purification. MS: (ES/+) m/z: 214 (M+1), 158 [M+1−C(CH₃)₃)] and 114 (M+1−Boc). C₁₁H₁₉NO₃ requires 213. ¹H-NMR (400 MHz, CDCl₃) δ (ppm): 5.61-5.82 (m, 2H), 4.35-4.64 (m, 1H), 3.98-4.30 (m, 1H), 3.48-3.73 (m, 3H), 2.35-2.48 (m, 1H), 1.96-2.15 (m, 1H), 1.50 (m, 9H).

### Description 3

### 1,1-dimethylethyl (2S)-2-({[(1,1-dimethylethyl)(diphenyl)silyl]oxy}methyl)-3,6-dihydro-1(2H)-pyridinecarboxylate (D3)

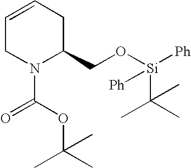

To a solution of 1,1-dimethylethyl (2S)-2-(hydroxymethyl)-3,6-dihydro-1(2H)-pyridinecarboxylate D2 (0.98 g of the crude material obtained in the Description 2) in DMF (5 ml), imidazole (1.56 g, 22.97 mmol) and chloro(1,1-dimethylethyl)diphenylsilane (1.52 g, 5.52 mmol) were added and the reaction mixture was left under stirring at room temperature for 3 hours. The mixture was diluted with brine and extracted with EtOAc. The organic phase was separated, dried (Na₂SO₄), filtered through a phase separator tube and concentrated under reduced pressure. The residue was purified by flash chromatography on silica gel (Flash Master 70 g, Cy/EtOAc 90/10) to afford the title compound D3 (1.81 g). MS: (ES/+) m/z: 452 (M+1) and 474 (M+Na). C₂₇H₃₇NO₃Si requires 451. ¹H-NMR (400 MHz, CDCl₃) δ (ppm): 7.57-7.78 (m, 4H), 7.32-7.51 (m, 6H), 5.44-5.75 (m, 2H), 4.37-4.80 (m, 1H), 4.02-4.31 (m, 1H), 3.53-3.72 (m, 2H), 3.28-3.51 (m, 1H), 1.99-2.44 (m, 2H), 1.48 (s, 9H), 1.07 (s, 9H).

### Description 4

### (2S)-2-({[(1,1-dimethylethyl)(diphenyl)silyl]oxy}methyl)-1,2,3,6-tetrahydropyridine (D4)

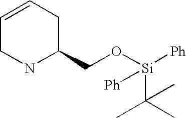

To a solution of 1,1-dimethylethyl (2S)-2-({[(1,1-dimethylethyl)(diphenyl)silyl]oxy}methyl)-3,6-dihydro-1(2H)-pyridinecarboxylate D3 (1.81 g) in DCM (40 ml), TFA (20 ml) was added and the reaction mixture stirred at room temperature for 1 hour. Volatiles were removed under reduced pressure and the residue was eluted through a SCX column. Collected fractions gave the title compound D4 (1.35 g). MS: (ES/+) m/z: 352 (M+1). C₂₂H₂₉NOSi requires 351. ¹H-NMR (300 MHz, CDCl₃) δ (ppm): 7.57-7.78 (m, 4H), 7.32-7.51 (m, 6H), 5.71-5.76 (m, 2H), 3.54-3.72 (m, 2H), 3.34-3.53 (m, 2H), 2.89-3.02 (m, 1H), 1.83-1.92 (m, 2H), 1.07 (s, 9H).

### Description 5A and 5B

### (2S)-2-({[(1,1-dimethylethyl)(diphenyl)silyl]oxy}methyl)-1-[(4-methylphenyl)sulfonyl]-1,2,3,6-tetrahydropyridine (D5A/D5B)

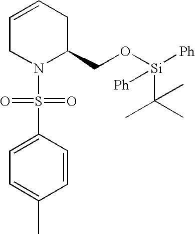

A) To a solution of (2S)-2-({[(1,1-dimethylethyl)(diphenyl)silyl]oxy}methyl)-1,2,3,6-tetrahydropyridine D4 (1.35 g) in DCM (25.60 ml), TEA (1.07 ml, 7.68 mmol) and 4-methylbenzenesulfonyl chloride (0.80 g, 4.22 mmol) were added and the resulting reaction mixture was stirred at room temperature overnight. The mixture was washed with a saturated aqueous NH₄Cl solution. The organic layer was separated, dried (Na₂SO₄), filtered through a phase separator tube and concentrated under reduced pressure. The residue was purified by flash chromatography on silica gel (Biotage SP 40 M, from Cy 100 to Cy/EtOAc 90/10) to afford the title compound D5A (1.90 g). MS: (ES/+) m/z: 506 (M+1) and 528 (M+Na). C₂₉H₃₅NO₃SSi requires 505. ¹H-NMR (300 MHz, CDCl₃) δ (ppm): 7.29-7.76 (m, 12H), 7.15 (d, 2H), 5.45-5.67 (m, 2H), 4.42-4.37 (m, 1H), 3.92-4.11 (m, 1H), 3.51-3.61 (m, 2H), 3.35-3.50 (m, 1H), 2.37 (s, 3H), 2.04-2.33 (m, 2H), 1.03 (s, 9H).

B) An alternative method to make D5 is as follows: N-[(1S)-1-({[(1,1-dimethylethyl)(diphenyl)silyl]oxy}methyl)-3-buten-1-yl]-4-methyl-N-2-propen-1-ylbenzenesulfonamide D9 (7.46 g) was dissolved in DCM (50 ml) then Grubbs I (1.170 g, 1.398 mmol) was added and the mixture was stirred at room temperature overnight. All volatiles were removed under vacuum and the resulting crude product was purified by silica gel chromatography (Biotage SP—column size 340 g SNAP, Cy to Cy/EtOAc 80/20) to afford the title compound D5B (7.4 g). MS: (ES/+) m/z: 506 (M+1) and 528 (M+Na). C₂₉H₃₅NO₃SSi requires 505. ¹H-NMR (400 MHz, CDCl₃) δ (ppm): 7.67-7.58 (m, 5H), 7.47-7.35 (m, 5H), 7.21-7.16 (m 2H), 5.5-4.8 (m, 2H), 4.42-4.37 (m, 1H), 4.11-3.92 (m, 1H), 3.62-3.50 (m, 2H), 3.50-3.35 (m, 1H), 2.40 (s, 3H), 2.33-2.11 (m, 2H), 2.00-1.08 (m, 2H), 1.05 (s, 9H).

### Description 6

### (1R,4S,6R)-4-({[(1,1-dimethylethyl)(diphenyl)silyl]oxy}methyl)-3-[(4-methylphenyl)sulfonyl]-3-azabicyclo[4.1.0]heptane (D6)

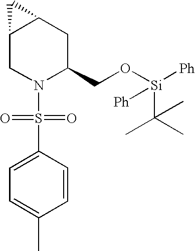

A solution of diethylzinc (1 M solution in hexanes, 21.35 ml, 21.35 mmol) in DCM (10 ml) was cooled down to 0° C. and TFA (1.64 ml, 21.35 mmol) was added dropwise. After 20 minutes stirring, diiodomethane (1.73 mol, 21.35 mmol) was added and the mixture left stirring for a further 20 minutes. A solution of (2S)-2-({[(1,1-dimethylethyl)(diphenyl)silyl]oxy}methyl)-1-[(4-methylphenyl)sulfonyl]-1,2,3,6-tetrahydropyridine D5A (1.35 g) in DCM (5 ml) was then added, the resulting reaction mixture was allowed to warm up to room temperature and stirred for 6 hours. A solution of diethylzinc (8 eq), TFA (8 eq) and diiodomethane (8 eq) in DCM was prepared and added to the previous mixture at 0° C. The resulting reaction mixture was left under stirring at room temperature overnight and washed with a saturated aqueous NH₄Cl solution. The aqueous layer was back-extracted with EtOAc. The collected organic layers were dried (Na₂SO₄), filtered through a phase separator tube and concentrated under reduced pressure. The residue was purified by flash chromatography on silica gel (Biotage SP 40 M, from Cy 100 to Cy/EtOAc 90/10) to afford the title compound D6 (0.83 g). MS: (ES/+) m/z: 520 (M+1) and 542 (M+Na). C₃₀H₃₇NO₃SSi requires 519. ¹H-NMR (300 MHz, CDCl₃) δ (ppm): 7.50-7.75 (m, 6H), 7.28-7.49 (m, 6H), 7.15 (d, 2H), 3.78-3.90 (m, 1H), 3.52-3.70 (m, 2H), 3.20-3.41 (m, 2H), 2.37 (s, 3H), 2.17-2.29 (m, 1H), 1.31-1.41 (m, 1H), 1.03 (s, 9H), 0.56-0.93 (m, 3H), −0.01 (q, 1H).

### Description 7

### N-[(1S)-1-(hydroxymethyl)-3-buten-1-yl]-4-methylbenzenesulfonamide (D7)

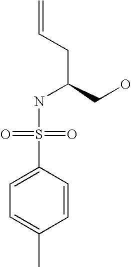

A solution of (2S)-2-amino-4-pentenoic acid (5 g, 43.4 mmol) in THF (200 ml) was cooled down to 0° C. and LiAlH₄ (1 M solution in THF, 54.3 ml, 54.3 mmol) was added dropwise. The resulting reaction mixture was allowed to warm-up to room temperature and stirred overnight. The mixture was then cooled down to 0° C. and quenched with a 2 M aqueous NaOH solution. The solid was filtered off and extracted with boiling THF for 1 hour. The combined ethereal extracts were concentrated under reduced pressure and the remaining aqueous mixture extracted with DCM. The combined organic phases were washed with brine, dried (Na₂SO₄) and evaporated under reduced pressure to afford the crude intermediate (2S)-2-amino-4-penten-1-ol (3.82 g) that was used in the next step without any further purification. A solution of sodium carbonate (6.40 g, 60.4 mmol) in water (35 ml) was left under stirring for 20 minutes at room temperature. (2S)-2-amino-4-penten-1-ol (3.82 g) was added, followed by EtOAc (80 ml). After 30 minutes stirring, a solution of p-toluenesulfonyl chloride (5.59 g, 29.3 mmol) in EtOAc (10 ml) and THF (10 ml) was added over 30 minutes. The reaction mixture was stirred at room temperature for 5 hours. Water (30 ml) and EtOAc (100 ml) were then added. The organic phase was separated and the aqueous one extracted with EtOAc (2×50 ml). The combined organic layers were dried (Na₂SO₄), filtered and concentrated under reduced pressure. The residue was purified by flash chromatography on silica gel (Biotage SP 340 g SNAP, from Cy/EtOAc 70/30 to EtOAc 100) to afford the title compound D7 (4.23 g). MS: (ES/+) m/z: 256 (M+1). C₁₂H₁₇NO₃S requires 255. ¹H-NMR (400 MHz, DMSO-d₆) δ (ppm): 7.68 (d, 2H), 7.48 (d, 1H), 7.37 (d, 2H), 5.48-5.63 (m, 1H), 4.82-4.98 (m, 2H), 4.66 (t, 1H), 3.18-3.27 (m, 1H), 3.00-3.17 (m, 2H), 2.39 (s, 3H), 2.17-2.27 (m, 1H), 1.91-2.03 (m, 1H).

### Description 8

### N-[(1S)-1-({[(1,1-dimethylethyl)(diphenyl)silyl]oxy}methyl)-3-buten-1-yl]-4-methylbenzenesulfonamide (D8)

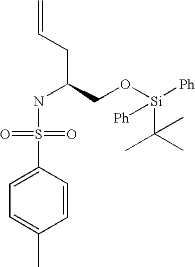

To a solution of N-[(1S)-1-(hydroxymethyl)-3-buten-1-yl]-4-methylbenzenesulfonamide D7 (4.23 g) in DMF (35 ml), imidazole (2.98 g, 43.7 mmol) and TBDPSCl (7.49 ml, 29.2 mmol) were added and the resulting reaction mixture was left under stirring overnight at room temperature. The mixture was diluted with H₂O (300 ml) and extracted with EtOAc (5×50 ml). The combined organic phases were dried (Na₂SO₄), filtered and concentrated under reduced pressure to give a yellow oil. The residue was purified by flash chromatography on silica gel (Biotage SP 340 g SNAP, from Cy 100 to Cy/EtOAc 90/10) to afford the title compound D8 (8.07 g) as a crude material which was used in the next step without any further purification. MS: (ES/+) m/z: 494 (M+1) and 516 (M+Na). C₂₈H₃₅NO₃SSi requires 493. ¹H-NMR (400 MHz, CDCl₃) δ (ppm): 7.69 (d, 2H), 7.35-7.77 (m, 10H), 7.24 (d, 2H), 5.47-5.63 (m, 1H), 5.01 (bs, 1H), 4.96-5.00 (m, 1H), 4.77 (bd, 1H), 3.57 (dd, 1H), 3.44 (dd, 1H), 3.25-3.37 (m, 1H), 2.43 (s, 3H), 2.30-2.37 (m, 2H), 1.05 (s, 9H).

### Description 9

### N-[(1S)-1-({[(1,1-dimethylethyl)(diphenyl)silyl]oxy}methyl)-3-buten-1-yl]-4-methyl-N-2-propen-1-ylbenzenesulfonamide (D9)

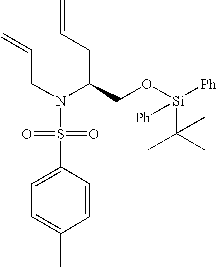

To a solution of N-[(1S)-1-({[(1,1-dimethylethyl)(diphenyl)silyl]oxy}methyl)-3-buten-1-yl]-4-methylbenzenesulfonamide D8 (8.07 g of the crude material obtained in the Description 8) in DMF (30 ml), cesium carbonate (7.46 g, 22.9 mmol) and 3-bromo-1-propene (1.38 g, 11.4 mmol) were added and the mixture was stirred at room temperature overnight. The mixture was diluted with H₂O (300 ml) and extracted with Et₂O (5×50 ml). The combined organic phases were dried (Na₂SO₄), filtered and concentrated under reduced pressure. The residue was purified by flash chromatography on silica gel (Biotage SP 340 g SNAP, from Cy 100 to Cy/EtOAc 90/10) to afford the title compound D9 (7.46 g). MS: (ES/+) m/z: 534 (M+1) and 556 (M+Na). C₃₁H₃₉NO₃SSi requires 533. ¹H-NMR (400 MHz, CDCl₃) δ (ppm): 7.35-7.79 (m, 12H), 7.20 (d, 2H), 5.72-5.86 (m, 1H), 5.47-5.62 (m, 1H), 4.88-5.16 (m, 4H), 3.90-4.05 (m, 2H), 3.77-3.88 (m, 1H), 3.59-3.71 (m, 2H), 2.40 (s, 3H), 2.38-2.51 (m, 1H), 2.22-2.33 (m, 1H), 1.04 (s, 9H).

### Description 10

### {(1R,4S,6R)-3-[(4-methylphenyl)sulfonyl]-3-azabicyclo[4.1.0]hept-4-yl}methanol (D10)

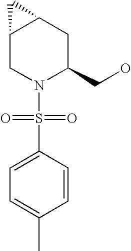

A solution of (1R,4S,6R)-4-({[(1,1-dimethylethyl)(diphenyl)silyl]oxy}methyl)-3-[(4-methylphenyl)sulfonyl]-3-azabicyclo[4.1.0]heptane D6 (0.83 g) in pyridine (8 ml) was cooled down to 0° C. and then hydrogen fluoride-pyridine (2.22 ml, 25.50 mmol) was added dropwise. The reaction mixture was left under stirring for 3 hours at room temperature. The mixture was washed with a saturated aqueous NH₄Cl solution and extracted with DCM. The organic layer was dried (Na₂SO₄), filtered through a phase separator tube and concentrated under reduced pressure. The residue was purified by flash chromatography on silica gel (Biotage SP 40 M, from Cy 100 to Cy/EtOAc 50/50) to afford the title compound D10 (0.36 g). UPLC (walk-up): rt=4.36 min. ¹H-NMR (500 MHz, CDCl₃) δ (ppm): 7.70 (d, 2H), 7.30 (d, 2H), 3.71-3.88 (m, 2H), 3.52-3.67 (m, 2H), 3.41 (dd, 1H), 2.43 (s, 3H), 1.83-1.98 (m, 2H), 1.37-1.48 (m, 1H), 0.95-1.03 (m, 1H), 0.84-0.94 (m, 1H), 0.63-0.72 (m, 1H), −0.05 (q, 1H).

### Description 11

### (1R,4S,6R)-3-[(4-methylphenyl)sulfonyl]-3-azabicyclo[4.1.0]heptane-4-carbaldehyde (D11)

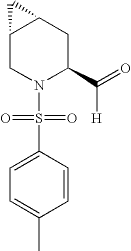

To a solution of {(1R,4S,6R)-3-[(4-methylphenyl)sulfonyl]-3-azabicyclo[4.1.0]hept-4-yl}methanol D10 (0.32 g) in DCM (8 ml), sodium bicarbonate (0.38 g, 4.55 mmol) and Dess-Martin periodinane (0.63 g, 1.48 mmol) were added and the resulting reaction mixture was stirred at room temperature for 1 hour. The mixture was washed with a saturated aqueous NH₄Cl solution. The organic layer was dried (Na₂SO₄), filtered through a phase separator tube and concentrated under reduced pressure. The residue was purified by flash chromatography on silica gel (Biotage SP 25 M, Cy/EtOAc 80/20) to afford the title compound D11 (0.19 g). UPLC (walk-up): rt=5.00 min. ¹H-NMR (400 MHz, CDCl₃) δ (ppm): 9.59 (s, 1H), 7.70 (d, 2H), 7.34 (d, 2H), 4.06 (m, 1H), 3.74 (m, 1H), 3.40 (m, 1H), 2.45-2.56 (m, 1H), 2.46 (s, 3H), 1.48-1.57 (m, 1H), 0.89-1.07 (m, 2H), 0.64-0.72 (m, 1H), −0.02 (q, 1H).

### Description 12

### (N-((1E)-{(1R,4S,6R)-3-[(4-methylphenyl)sulfonyl]-3-azabicyclo[4.1.0]hept-4-yl}methylidene)-5-(trifluoromethyl)-2-pyridinamine (D12)

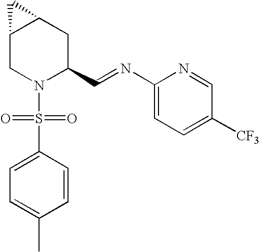

AcOH (0.12 ml, 2.04 mmol) was added to a solution of (1R,4S,6R)-3-[(4-methylphenyl)sulfonyl]-3-azabicyclo[4.1.0]heptane-4-carbaldehyde D11 (0.19 g) and 5-(trifluoromethyl)-2-pyridinamine (available from Sigma-Aldrich #684716) (0.13 g, 0.82 mmol) in 1,2-DCE (3 ml) and the mixture was stirred at room temperature for 1 hour. Sodium triacetoxyborohydride (0.20 g, 0.95 mmol) was then added and the resulting mixture stirred for 2 hour. The mixture was diluted with DCM (5 ml) and washed with brine. The organic phase was separated, dried (Na₂SO₄), filtered through a phase separator tube and concentrated under reduced pressure The residue was purified by flash chromatography on silica gel (Biotage SP 25 M, Cy/EtOAc 70/30) to afford the title compound D12 (0.10 g). MS: (ES/+) m/z: 424 (M+1). C₂₀H₂₀F₃N₃O₂S requires 423.

### Description 13

### N-({(1R,4S,6R)-3-[(4-methylphenyl)sulfonyl]-3-azabicyclo[4.1.0]hept-4-yl}methyl)-5-(trifluoromethyl)-2-pyridinamine (D13)

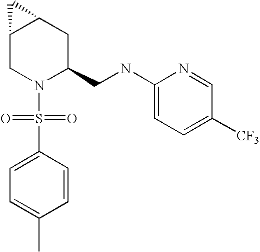

To a solution of (N-((1E)-{(1R,4S,6R)-3-[(4-methylphenyl)sulfonyl]-3-azabicyclo[4.1.0]hept-4-yl}methylidene)-5-(trifluoromethyl)-2-pyridinamine D12 (0.10 g) in 1,2-DCE (3 ml), AcOH (0.041 ml, 0.71 mmol) and sodium triacetoxyborohydride (0.15 g, 0.71 mmol) were added and the resulting mixture was left under stirring overnight at room temperature. The mixture was diluted with DCM (5 ml) and washed with a saturated aqueous NaHCO₃ solution. The organic phase was separated, dried (Na₂SO₄), filtered through a phase separator tube and concentrated under reduced pressure The residue was purified by flash chromatography on silica gel (Biotage SP 25 M, Cy/EtOAc 70/30) to afford the title compound D13 (0.063 g). MS: (ES/+) m/z: 426 (M+1). C₂₀H₂₂F₃N₃O₂S requires 425. ¹H-NMR (400 MHz, CDCl₃) δ (ppm): 8.32 (bs, 1H), 7.64-7.71 (m, 2H), 7.54 (dd, 1H), 7.25-7.32 (m, 2H), 6.41 (d, 1H), 5.21 (bs, 1H), 3.93-4.02 (m, 1H), 3.66-3.77 (m, 1H), 3.52-3.57 (m, 2H), 3.42-3.51 (m, 1H), 2.43 (s, 3H), 1.88-1.98 (m, 1H), 1.41-1.53 (m, 1H), 0.86-1.07 (m, 2H), 0.65-0.76 (m, 1H), −0.13 (q, 1H).

### Description 14

### N-[(1R,4S,6R)-3-azabicyclo[4.1.0]hept-4-ylmethyl]-5-(trifluoromethyl)-2-pyridinamine (D14)

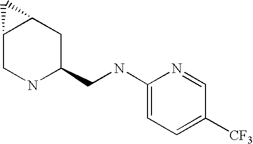

Naphthalene (0.95 g, 7.40 mmol) was added to a solution of sodium (0.17 g, 7.40 mmol) in anhydrous THF (40 ml) and the mixture was stirred at room temperature for 1 hour to afford an approximately 0.2 M deep green sodium naphthalenide solution. 1.5 ml (approximately 3 mmol) of this freshly prepared solution were carefully added at −78° C. to a solution of N-({(1R,4S,6R)-3-[(4-methylphenyl)sulfonyl]-3-azabicyclo[4.1.0]hept-4-yl}methyl)-5-(trifluoromethyl)-2-pyridinamine D13 (0.063 g) in THF (3 ml). After 30 minutes stirring at −78° C. a further 3 ml (approximately 6 mmol) of the sodium naphthalenide solution were added and the reaction mixture was left stirring overnight. A further 7.5 ml (approximately 1.50 mmol) of the sodium naphthalenide solution were added and the reaction mixture stirred for 15 minutes. Water was added and the mixture extracted with EtOAc. The organic phase was separated, dried (Na₂SO₄), filtered through a phase separator tube and concentrated under reduced pressure The residue was eluted through a SCX column (10 g) to afford the title compound D14 (0.040 g). UPLC (walk-up): 3.68 min. ¹H-NMR (500 MHz, CDCl₃) δ (ppm): 8.32 (d, 1H), 7.54 (dd, 1H), 6.43 (d, 1H), 5.49-5.66 (m, 1H), 3.38-3.67 (m, 2H), 3.01-3.15 (m, 1H), 2.77 (dd, 1H), 2.44-2.55 (m, 1H), 1.93 (dd, 1H), 1.55-1.68 (m, 1H), 0.97-1.16 (m, 2H), 0.64-0.78 (m, 1H), 0.22 (q, 1H).

### Description 15

### 2-({(1R,4S,6R)-3-[(4-methylphenyl)sulfonyl]-3-azabicyclo[4.1.0]hept-4-yl}methyl)-1H-isoindole-1,3(2H)-dione (D15)

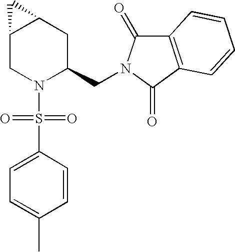

A mixture of {(1R,4S,6R)-3-[(4-methylphenyl)sulfonyl]-3-azabicyclo[4.1.0]hept-4-yl}methanol D10 (2 g), triphenylphosphine (2.80 g, 10.66 mmol) and phthalimide (1.25 g, 8.53 mmol) in THF (2 ml) was heated to 50° C. and then DIAD (2.07 ml, 10.66 mmol) was added dropwise. The resulting reaction mixture was stirred for 30 minutes at 50° C. and then water (0.2 ml) was added. Volatiles were removed under reduced pressure and the reaction crude was purified by flash chromatography on silica gel (Flash Master Personal 5 g, Cy/EtOAc 80/20) to afford the title compound D15 (2.20 g). MS: (ES/+) m/z: 411 (M+1). C₂₂H₂₂N₂O₄S requires 410. ¹H-NMR (400 MHz, CDCl₃) δ (ppm): 7.76-7.82 (m, 2H), 7.68-7.85 (m, 2H), 7.52-7.57 (m, 2H), 6.98-7.03 (m, 2H), 4.21-4.31 (m, 1H), 4.03-4.19 (m, 1H), 3.88 (dd, 1H), 3.73-3.83 (m, 1H), 3.53 (dd, 1H), 2.22 (s, 3H), 1.86-1.96 (m, 1H), 1.65-1.76 (m, 1H), 0.97-1.16 (m, 2H), 0.71-0.81 (m, 1H), 0.04 (q, 1H).

### Description 16

### ({(1R,4S,6R)-3-[(4-methylphenyl)sulfonyl]-3-azabicyclo[4.1.0]hept-4-yl}methyl)amine (D16)

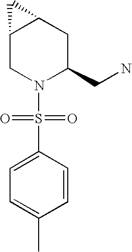

To a solution of 2-({(1R,4S,6R)-3-[(4-methylphenyl)sulfonyl]-3-azabicyclo[4.1.0]hept-4-yl}methyl)-1H-isoindole-1,3(2H)-dione D15 (2.20 g) in EtOH (50 ml), hydrazine monohydrate (3.28 ml, 53.60 mmol) was carefully added and the resulting reaction mixture stirred at room temperature overnight. Volatiles were removed under reduced pressure and the solid residue was purified by flash chromatography on silica gel (Biotage SP 40 M, from EtOAc to DCM/MeOH 95/5) to afford the title compound D16 (1.30 g). MS: (ES/+) m/z: 281 (M+1). C₁₄H₂₀N₂O₂S requires 280. ¹H-NMR (400 MHz, CDCl₃) δ (ppm): 7.70 (d, 2H), 7.31 (d, 2H), 3.68-3.79 (m, 1H), 3.55-3.65 (m, 1H), 3.37-3.48 (m, 1H), 2.89 (dd, 1H), 2.70 (dd, 1H), 2.44 (s, 3H), 1.83-1.96 (m, 1H), 1.47-1.70 (m, 1H), 0.80-0.97 (m, 2H), 0.58-0.69 (m, 1H), −0.18 (q, 1H).

### Description 17

### 6-[({(1R,4S,6R)-3-[(4-methylphenyl)sulfonyl]-3-azabicyclo[4.1.0]hept-4-yl}methyl)amino]-3-pyridinecarbonitrile (D17)

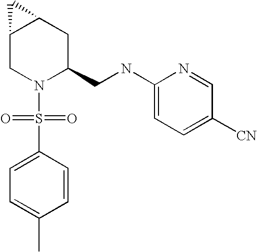

To a solution of ({(1R,4S,6R)-3-[(4-methylphenyl)sulfonyl]-3-azabicyclo[4.1.0]hept-4-yl}methyl)amine D16 (0.10 g) in DMSO (2 ml), DIPEA (0.12 ml, 0.71 mmol) and 6-chloro-3-pyridinecarbonitrile (available from Sigma-Aldrich #510734) (0.0593 g, 0.43 mmol) were added. The resulting reaction mixture was stirred at 120° C. for 4 hours, diluted with a saturated aqueous NH₄Cl solution and extracted with EtOAc. The organic phase was separated, dried (Na₂SO₄), filtered through a phase separator tube and concentrated under reduced pressure. The residue was purified by flash chromatography on silica gel (Biotage SP 25 M, Cy/EtOAc 70/30) to afford the title compound D17 (0.085 g). MS: (ES/+) m/z: 383 (M+1). C₂₀H₂₂N₄O₂S requires 382. ¹H-NMR (400 MHz, CDCl₃) δ (ppm): 8.36-8.40 (m, 1H), 7.64-7.71 (m, 2H), 7.55 (dd, 1H), 7.25-7.35 (m, 2H), 6.43 (d, 1H), 5.49 (bs, 1H), 3.91-4.04 (m, 1H), 3.69-3.84 (m, 1H), 3.40-3.63 (m, 3H), 2.45 (s, 3H), 1.86-1.99 (m, 1H), 1.39-1.52 (m, 1H), 0.89-1.09 (m, 2H), 0.66-0.78 (m, 1H), −0.17 (q, 1H).

### Description 18

### 6-{[(1R,4S,6R)-3-azabicyclo[4.1.0]hept-4-ylmethyl]amino}-3-pyridinecarbonitrile (D18)

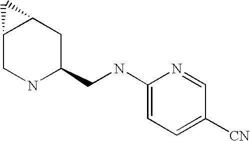

Naphthalene (1.42 g, 11.11 mmol) was added to a solution of sodium (0.25 g, 11.11 mmol) in anhydrous THF (22 ml) and the mixture was stirred at room temperature for 1 hour to afford an approximately 0.5 M deep green sodium naphthalenide solution. 7 ml (approximately 3.50 mmol) of this freshly prepared solution were carefully added at −78° C. to a solution of 6-[({(1R,4S,6R)-3-[(4-methylphenyl)sulfonyl]-3-azabicyclo[4.1.0]hept-4-yl}methyl)amino]-3-pyridinecarbonitrile D17 (0.085 g) in THF (2 ml). After 30 minutes stirring the reaction mixture was diluted with water and extracted with EtOAc. The organic phase was separated, dried (Na₂SO₄), filtered through a phase separator tube and concentrated under reduced pressure The residue was eluted through a SCX column (10 g) to afford the title compound D18 (0.043 g). MS: (ES/+) m/z: 229 (M+1). C₁₃H₁₆N₄ requires 228. ¹H-NMR (400 MHz, CDCl₃) δ (ppm): 8.35 (d, 1H), 7.51 (dd, 1H), 6.40 (d, 1H), 5.88 (bs, 1H), 3.42-3.61 (m, 2H), 2.99-3.14 (m, 1H), 2.75 (dd, 1H), 2.40-2.51 (m, 1H), 1.92 (dd, 1H), 1.51-1.64 (m, 1H), 0.97-1.19 (m, 2H), 0.68-0.77 (m, 1H), 0.21 (q, 1H).

### Description 19

### 4,6-dimethyl-N-({(1R,4S,6R)-3-[(4-methylphenyl)sulfonyl]-3-azabicyclo[4.1.0]hept-4-yl}methyl)-2-pyrimidinamine (D19)

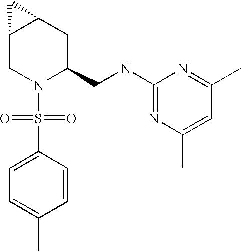

To a solution of ({(1R,4S,6R)-3-[(4-methylphenyl)sulfonyl]-3-azabicyclo[4.1.0]hept-4-yl}methyl)amine D16 (0.10 g) in DMSO (2 ml), DIPEA (0.075 ml, 0.43 mmol) and 2-chloro-4,6-dimethylpyrimidine (available from Alfa Aesar #H50331) (0.061 g, 0.43 mmol) were added. The resulting reaction mixture was stirred at 100° C. overnight, diluted with water (10 ml) and extracted with DCM. The collected organic phases were dried (Na₂SO₄), filtered through a phase separator tube and concentrated under reduced pressure. The reaction crude was purified by flash chromatography on silica gel (Biotage SP SNAP 25 g, from Cy/EtOAc 70/30 to Cy/EtOAc 30/70) to afford the title compound D19 (0.060 g). MS: (ES/+) m/z: 387 (M+1). C₂₀H₂₆N₄O₂S requires 386. ¹H NMR (400 MHz, CDCl₃) δ ppm 7.67-7.75 (m, 2H), 7.20-7.26 (m, 2H), 6.28-6.34 (m, 1H), 4.97-5.08 (m, 1H), 3.98-4.08 (m, 1H), 3.65-3.71 (m, 1H), 3.45-3.58 (m, 2H), 2.38-2.43 (m, 3H), 2.29 (s, 6H), 1.92-2.00 (m, 1H), 1.52-1.61 (m, 1H), 0.94-1.03 (m, 2H), 0.65-0.76 (m, 1H), 0.02-0.10 (m, 1H).

### Description 20

### N-[(1R,4S,6R)-3-azabicyclo[4.1.0]hept-4-ylmethyl]-4,6-dimethyl-2-pyrimidinamine (D20)

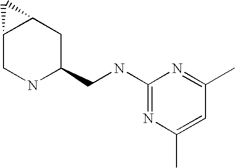

Sodium (0.0357 g, 1.55 mmol) was added to a solution of naphthalene (0.20 g, 1.55 mmol) in anhydrous THF (10 ml) and the mixture was stirred at room temperature for 2 hours to afford a deep green sodium naphthalenide solution. The freshly prepared solution was added at −78° C. to a solution of 4,6-dimethyl-N-({(1R,4S,6R)-3-[(4-methylphenyl)sulfonyl]-3-azabicyclo[4.1.0]hept-4-yl}methyl)-2-pyrimidinamine D19 (0.060 g) in THF (3 ml). After 30 minutes stirring at −78° C., the reaction mixture was quenched with water (0.50 ml) and allowed to warm up to room temperature. Volatiles were removed under reduced pressure. The residue was eluted through a SCX column (5 g) to afford the title compound D20 (0.035 g). MS: (ES/+) m/z: 233 (M+1). C₁₃H₂₀N₄ requires 232. ¹H NMR (400 MHz, CDCl₃) δ ppm 6.27 (s, 1H), 5.52-5.64 (m, 1H), 3.33-3.55 (m, 4H), 3.14-3.33 (m, 2H), 2.67-2.78 (m, 1H), 2.42-2.52 (m, 1H), 2.14-2.42 (m, 10H), 1.96-2.05 (m, 1H), 1.86-1.94 (m, 1H), 1.55-1.68 (m, 1H), 1.18-1.37 (m, 2H), 0.97-1.10 (m, 3H), 0.60-0.74 (m, 1H), 0.12-0.25 (m, 1H).

### Description 21

### (1R,4S,6R)-4-({[(1,1-dimethylethyl)(diphenyl)silyl]oxy}methyl)-3-azabicyclo[4.1.0]heptane (D21)

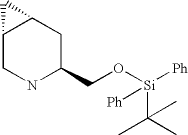

To a solution of (1R,4S,6R)-4-({[(1,1-dimethylethyl)(diphenyl)silyl]oxy}methyl)-3-[(4-methylphenyl)sulfonyl]-3-azabicyclo[4.1.0]heptane D6 (3.6 g) in MeOH (500 ml), under a nitrogen atmosphere, magnesium (9.76 g, 402 mmol) (turnings, previously flame dried) and NH₄Cl (10.37 g, 194 mmol) were subsequently added and the reaction mixture was vigorously stirred at 23° C. After 2 hours further Mg (5 g) was added and the reaction mixture was stirred for other 2.5 hours. About 25% of starting material was present and DCM (300 ml) and aqueous NH₄Cl (saturated solution 200 ml) were added. The organic layer was separated and washed with brine (80 ml), filtered through a hydrophobic filter and evaporated under reduced pressure to give a colorless oil which was charged on a SCX (20 g) to afford the title compound D21 (1.81 g). UPLC (Acid IPQC): rt1=1.00 minutes, peaks observed: 365 (M+1). C₂₃H₃₁NOSi requires 364. ¹H NMR (400 MHz, DMSO-d6) δ ppm 0.11-0.19 (m, 1H) 0.50-0.60 (m, 1H) 0.86-1.07 (m, 11H) 1.40-1.56 (m, 2H) 1.63-1.75 (m, 1H) 2.23-2.37 (m, 1H) 2.55-2.65 (m, 1H) 3.43-3.51 (m, 2H) 7.36-7.51 (m, 6H) 7.55-7.67 (m, 4H).

### Description 22

### (1R,4S,6R)-4-({[(1,1-dimethylethyl)(diphenyl)silyl]oxy}methyl)-3-{[6-methyl-3-(2-pyrimidinyl)-2-pyridinyl]carbonyl}-3-azabicyclo[4.1.0]heptane (D22)

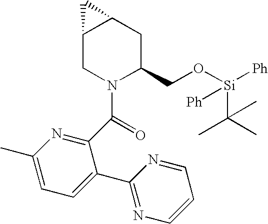

To a solution of (1R,4S,6R)-4-({[(1,1-dimethylethyl)(diphenyl)silyl]oxy}methyl)-3-azabicyclo[4.1.0]heptane D21 (1.5 g) in dry DCM (30 ml) at room temperature under N₂ flux, 6-methyl-3-(2-pyrimidinyl)-2-pyridinecarboxylic acid D69 (2.94) and DIPEA (2.150 ml, 12.31 mmol) were added, followed by TBTU (1.534 g, 4.78 mmol), and the yellow suspension was left stirring at room temperature for 1.5 hour. Mixture was diluted with DCM and washed twice with NaHCO₃ saturated solution; aqueous phase was back-extracted with DCM and the collected organic phases were washed with water and brine. Organic layers were collected, dried over Na₂SO₄, filtered and evaporated; resulting dark green oil was purified by flash chromatography on KP-NH column (SNAP 110 g eluting with Cy/AcOEt 1:1) affording the title compound D22 (1.79 g) as light yellow oil. ¹H NMR (400 MHz, CDCl₃) δ ppm 0.46-0.54 (m, 1H) 0.56-0.65 (m, 1H) 0.73-0.98 (m, 2H) 1.11 (s, 9H) 1.79-1.87 (m, 1H) 2.42-2.50 (m, 1H) 2.60 (s, 3H) 3.21-4.30 (m, 4H) 4.66-4.78 (m, 1H) 7.01 (t, 1H) 7.19-7.80 (m, 11H) 8.42 (d, 2H) 8.49 (d, 1H)

### Description 23

### ((1R,4S,6R)-3-{[6-methyl-3-(2-pyrimidinyl)-2-pyridinyl]carbonyl}-3-azabicyclo[4.1.0]hept-4-yl)methanol (D23)

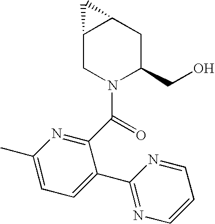

To a stirring solution of (1R,4S,6R)-4-({[(1,1-dimethylethyl)(diphenyl)silyl]oxy}methyl)-3-{[6-methyl-3-(2-pyrimidinyl)-2-pyridinyl]carbonyl}-3-azabicyclo[4.1.0]heptane D22 (1.79 g) in dry THF (25 ml) at room temperature under nitrogen flux, TBAF (3.50 ml, 3.50 mmol) was added slowly and the mixture was stirred at room temperature for 2 hours. Mixture was diluted with AcOEt and washed with NH₄Cl saturated solution and brine; organic phases collected were dried (Na₂SO₄), filtered and evaporated, and the resulting crude purified by flash chromatography (on KP-Sil SNAP 100 g column eluting with DCM/MeOH 95:5), affording the title compound D23 (600 mg) as white foam. A second batch of the title compound D23 (295 mg, 0.909 mmol, 28.6% yield) was obtained as slightly impure product. UPLC (Acid GEN_QC_SS): rt1=0.58 minutes, peaks observed: 325 (M+1). C₁₈H₂₀N₄O₂ requires 324. ¹H NMR (500 MHz, DMSO-d₆) δ ppm 8.80-8.94 (m, 2H), 8.43 (d, 1H), 7.39-7.52 (m, 2H), 4.74 (t, 1H), 4.22-4.30 (m, 1H), 3.50-3.71 (m, 2H), 3.45 (dd, 1H), 3.18-3.23 (m, 1H), 2.53-2.56 (m, 3H), 2.15-2.24 (m, 1H), 1.58-1.67 (m, 1H), 0.83-1.10 (m, 2H), 0.52-0.61 (m, 1H), 0.41-0.47 (m, 1H).

### Description 24

### 2-[((1R,4S,6R)-3-{[6-methyl-3-(2-pyrimidinyl)-2-pyridinyl]carbonyl}-3-azabicyclo[4.1.0]hept-4-yl)methyl]-1H-isoindole-1,3(2H)-dione (D24)

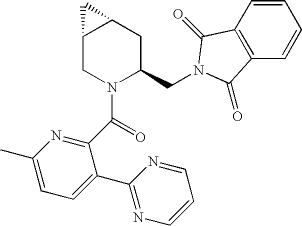

To a solution of ((1R,4S,6R)-3-{[6-methyl-3-(2-pyrimidinyl)-2-pyridinyl]carbonyl}-3-azabicyclo[4.1.0]hept-4-yl)methanol D23 (270 mg) in dry THF (7 ml), phthalimide (147 mg, 0.999 mmol) and triphenylphosphine (327 mg, 1.249 mmol) were added. Mixture was brought to 50° C., then DIAD (0.243 ml, 1.249 mmol) was added dropwise and the solution was stirred at the same temperature for 1 hour. After cooling to room temperature 0.1 ml of water were added and volatiles were evaporated under reduced pressure; resulting crude was purified by flash chromatography on (KP-Sil column SNAP 25 g eluting with AcOEt 100%), affording the title compound D24 (297 mg) as white solid. UPLC (Acid GEN_QC_SS): rt1=0.75 minutes and rt2=0.82 minutes (rotamers present), peaks observed: 454 (M+1). C₂₆H₂₃N₅O₃ requires 453. ¹H NMR (400 MHz, CDCl₃) δ ppm 0.37-0.43 (m, 1H) 0.81-0.91 (m, 1H) 0.98-1.31 (m, 2H) 1.69-2.16 (m, 2H) 2.43 (s, 3H) 3.63-3.99 (m, 3H) 4.23-4.37 (m, 1H) 4.60 (d, 1H) 7.03 (t, 1H) 7.25 (d, 1H) 7.61-7.92 (m, 4H) 8.51 (d, 1H) 8.56 (d, 2H).

### Description 25

### [((1R,4S,6R)-3-{[6-methyl-3-(2-pyrimidinyl)-2-pyridinyl]carbonyl}-3-azabicyclo[4.1.0]hept-4-yl)methyl]amine (D25)

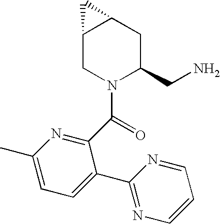

2-[((1R,4S,6R)-3-{[6-methyl-3-(2-pyrimidinyl)-2-pyridinyl]carbonyl}-3-azabicyclo[4.1.0]hept-4-yl)methyl]-1H-isoindole-1,3(2H)-dione D24 (297 mg) was dissolved in EtOH (7 ml), then hydrazine (0.206 ml, 6.55 mmol) was added and the mixture was stirred at room temperature overnight. The morning after a white solid was precipitated and TLC (DCM/MeOH 9:1) and UPLC showed reaction was complete. Solvent was removed at reduced pressure, the residue redissolved in MeOH and charged on a SCX cartridge (5 g) and the cartridge was eluted. Fractions containing desired product were evaporated and the residue purified by flash chromatography (on KP-NH column SNAP 11 g eluting with AcOEt 100%) affording the title compound D25 (150 mg) as white foam. ¹H NMR (500 MHz, DMSO-d₆) δ ppm 8.82-8.89 (m, 2H), 8.39 (d, 1H), 7.45-7.52 (m, 1H), 7.40-7.46 (m, 1H), 4.19-4.28 (m, 1H), 3.45 (d, 1H), 3.24 (d, 1H), 2.85-2.93 (m, 1H), 2.69-2.76 (m, 1H), 2.53 (s, 3H), 2.00-2.13 (m, 1H), 1.65-1.75 (m, 1H), 1.45-1.68 (m, 2H), 0.90-1.00 (m, 1H), 0.81-0.91 (m, 1H), 0.47-0.60 (m, 2H).

### Description 26

### 1,1-dimethylethyl(1R,4S,6R)-4-({[(1,1-dimethylethyl)(diphenyl)silyl]oxy}methyl)-3-azabicyclo[4.1.0]heptane-3-carboxylate (D26)

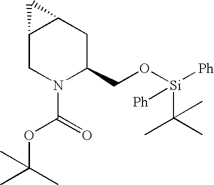

(1R,4S,6R)-4-({[(1,1-dimethylethyl)(diphenyl)silyl]oxy}methyl)-3-azabicyclo[4.1.0]heptane D21 (2 g) was dissolved in 100 ml of DCM then Boc₂O (1.270 ml, 5.47 mmol) and TEA (0.763 ml, 5.47 mmol) were added. The reaction was stirred at room temperature overnight. All volatiles were removed under vacuum and the residue was purified by silica gel chromatography (column size SNAP 100 g, using Cy:EtOAc=9:1 as eluent). It was recovered the title compound D26 (2.5 g). UPLC: (Basic Gen_QC): rt=1.33, peak observed: 466 (M+1). C₂₈H₃₉NO₃Si requires 465. ¹H NMR (400 MHz, CDCl₃) δ ppm 7.83-7.60 (m, 4H) 7.51-7.35 (m, 6H) 4.25-3.78 (m, 2H) 3.57-3.74 (m, 1H) 3.49-3.27 (m, 1H) 2.18-1.55 (m, 3H) 1.52-1.34 (m, 9H) 1.05 (s, 9H) 0.99-0.79 (m, 2H) 0.69-0.51 (m, 1H) 0.17-0.01 (m, 1H).

### Description D27

### 1,1-dimethylethyl(1R,4S,6R)-4-(hydroxymethyl)-3-azabicyclo[4.1.0]heptane-3-carboxylate (D27)

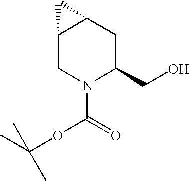

1,1-dimethylethyl(1R,4S,6R)-4-({[(1,1-dimethylethyl)(diphenyl)silyl]oxy}methyl)-3-azabicyclo[4.1.0]heptane-3-carboxylate D26 (2.5 g) was dissolved in THF (50 ml) then TBAF (5.37 ml, 5.37 mmol) was added and the reaction was stirred at room temperature overnight. All volatiles were removed under vacuum and the residue was purified by silica gel chromatography (column size—2×100 g SNAP, using Cy:EtOAc=8:2 to 2:8 as eluent). It was recovered the title compound D27 (1.25 g). UPLC: (Basic Gen_QC): rt=0.71, peak observed: 228 (M+1). C₁₂H₂₁NO₃ requires 227. ¹H NMR (400 MHz, CDCl₃) δ ppm 4.15-2.86 (m, 4H) 1.96-1.83 (m, 1H) 1.80-1.69 (m, 1H) 1.68-1.60 (m, 1H) 1.48 (s, 9H), 1.08-0.86 (m, 2H) 0.73-0.56 (m, 1H) 0.28-0.04 (m, 1H).

### Description D28

### 1,1-dimethylethyl(1R,4S,6R)-4-[(1,3-dioxo-1,3-dihydro-2H-isoindol-2-yl)methyl]-3-azabicyclo[4.1.0]heptane-3-carboxylate (D28)

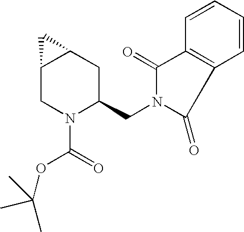

1,1-dimethylethyl(1R,4S,6R)-4-(hydroxymethyl)-3-azabicyclo[4.1.0]heptane-3-carboxylate D27 (1.43 g) was dissolved in THF (50 ml) then 1H-isoindole-1,3(2H)-dione (1.111 g, 7.55 mmol) and triphenylphosphine (2.475 g, 9.44 mmol) were added. This solution was warmed up to 50° C. and then DIAD (1.835 ml, 9.44 mmol) was added dropwise. The reaction was stirred at 50° C. for 30 minutes, then it was cooled to room temperature and all volatiles were removed under vacuum. The residue was purified by Silica Gel Chromatography (Biotage SP—column size 100 g SNAP) eluting with Cy:EtOAc=8:2 to 5:5 as eluent. It was recovered the title compound D28 (1.85 g). UPLC: (Acid Final_QC): rt=0.81, peak observed: 357 (M+1). C₂₀H₂₄N₂O₄ requires 356. ¹H NMR (400 MHz, DMSO-d₆) δ ppm 8.04-7.69 (m, 4H) 4.35-4.12 (m, 1H) 4.00-3.83 (m, 1H) 3.77-3.40 (m, 3H) 2.07-1.81 (m, 1H) 1.77-1.55 (m, 1H) 1.13-0.94 (m, 9H) 0.75-0.59 (m, 1H) 0.07-0.19 (m, 1H).

### Description D29

### 1,1-dimethylethyl(1R,4S,6R)-4-(aminomethyl)-3-azabicyclo[4.1.0]heptane-3-carboxylate (D29)

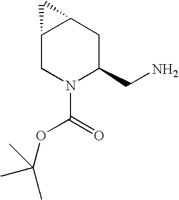

1,1-dimethylethyl(1R,4S,6R)-4-[(1,3-dioxo-1,3-dihydro-2H-isoindol-2-yl)methyl]-3-azabicyclo[4.1.0]heptane-3-carboxylate D28 (1.85 g) was dissolved in EtOH (20 ml) then hydrazine (2.036 ml, 51.9 mmol) was carefully added and the reaction stirred at room temperature overnight. All volatiles were removed under vacuum and the solid residue was triturated with Et₂O. These organic phases were collected together and concentrated to dryness to give the title compound D29 as pale yellow oil (1.1 g). UPLC: (Acid Final_QC): rt=0.45, peak observed: 227 (M+1). C₁₂H₂₂N₂O₂ requires 226. ¹H NMR (400 MHz, CDCl₃) δ ppm 4.09-3.64 (m, 2H) 3.41-3.18 (m, 1H) 2.99-2.83 (m, 1H) 2.77-2.59 (m, 1H) 1.90-1.71 (m, 2H) 1.67-1.48 (m, 2H) 1.47 (s, 1H) 1.02-0.84 (m, 2H) 0.74-0.55 (m, 1H) 0.18-0.18 (m, 1H).

### Description D30

### 1,1-dimethylethyl(1R,4S,6R)-4-({[6-(trifluoromethyl)-3-pyridazinyl]amino}methyl)-3-azabicyclo[4.1.0]heptane-3-carboxylate (D30)

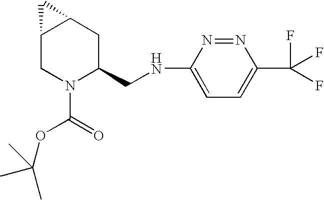

1,1-dimethylethyl(1R,4S,6R)-4-(aminomethyl)-3-azabicyclo[4.1.0]heptane-3-carboxylate D29 (45 mg) was dissolved in DMSO (1 ml) then DIPEA (0.038 ml, 0.219 mmol) and 3-chloro-6-(trifluoromethyl)pyridazine (39.9 mg, 0.219 mmol) were added and the mixture was stirred at 100° C. for 4 hours. NaHCO₃ saturated solution was added and the aqueous layer was extracted with DCM. The combined organic layers were dried over Na₂SO₄ anhydrous, filtered through a phase separator tube and concentrated under vacuum to give a crude product which was purified by Silica Gel Chromatography (Biotage SP—column size 25 g using Cy:EtOAc=9:1 to 5:5 as eluent). It was recovered the title compound D30 (53 mg). UPLC: (Acid Final_QC): rt=0.77, peak observed: 373 (M+1). C₁₇H₂₃F₃N₄O₂ requires 372. ¹H NMR (400 MHz, CDCl₃) δ ppm 7.49-7.36 (m, 1H) 6.82-6.66 (m, 1H) 6.19-6.03 (m, 1H) 4.46-4.19 (m, 1H) 4.12-3.80 (m, 1H) 3.74-3.41 (m, 3H) 2.13-1.90 (m, 1H) 1.87-1.75 (m, 1H) 1.51-1.43 (m, 9H) 1.13-0.97 (m, 2H) 0.81-0.65 (m, 1H) 0.27-0.03 (m, 1H).

### Description D31

### N-[(1R,4S,6R)-3-azabicyclo[4.1.0]hept-4-ylmethyl]-6-(trifluoromethyl)-3-pyridazinamine (D31)

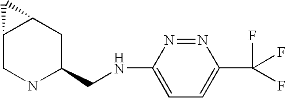

1,1-dimethylethyl(1R,4S,6R)-4-({[6-(trifluoromethyl)-3-pyridazinyl]amino}methyl)-3-azabicyclo[4.1.0]heptane-3-carboxylate D30 (53 mg) was dissolved in DCM (4 ml), TFA (2 ml, 26.0 mmol) was added and the mixture was stirred at room temperature for 6 hours. All volatiles were removed under vacuum and the residue was purified by SCX chromatography (column size 5 g). It was recovered the title compound D31 (36 mg). UPLC: (Acid Final_QC): rt=0.43, peaks observed: 273 (M+1). C₁₂H₁₅F₃N₄ requires 272. ¹H NMR (400 MHz, CDCl₃) δ ppm 7.38 (d, 1H,) 6.73 (d, 1H) 6.33 (br.s., 1H) 3.79-3.62 (m, 1H) 3.54-3.44 (m, 1H) 3.22-3.07 (m, 1H) 2.82-2.68 (m, 1H) 2.61-2.48 (m, 1H) 2.20-2.05 (br.s., 1H) 1.98-1.90 (m, 1H) 1.68-1.49 (m, 1H) 1.17-1.00 (m, 2H) 0.78-0.67 (m, 1H) 0.31-0.11 (m, 1H).

### Description D32

### 1,1-dimethylethyl(1R,4S,6R)-4-({[5-(trifluoromethyl)-2-pyrimidinyl]amino}methyl)-3-azabicyclo[4.1.0]heptane-3-carboxylate (D32)

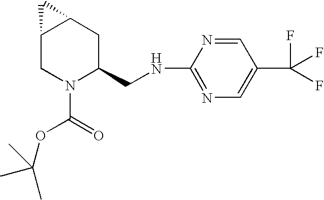

1,1-dimethylethyl(1R,4S,6R)-4-(aminomethyl)-3-azabicyclo[4.1.0]heptane-3-carboxylate D29 (45 mg) was dissolved in DMSO (1 ml) then DIPEA (0.038 ml, 0.219 mmol) and 2-chloro-5-(trifluoromethyl)pyrimidine (36.3 mg, 0.199 mmol) were added and the mixture was stirred at 100° C. for 4 hours. NaHCO₃ saturated solution (10 ml) was added and the aqueous layer was extracted with DCM. The combined organic layers were dried over Na₂SO₄ anhydrous, filtered through a phase separator tube and concentrated under vacuum to give a crude product which was purified by silica gel chromatography (Biotage SP—column size 25 g, using Cy:EtOAc=9:1 to 5:5 as eluent). It was recovered the title compound D32 (45 mg). UPLC: (Acid Final_QC): rt=0.87, peak observed: 373 (M+1). C₁₇H₂₃F₃N₄O₂ requires 372. ¹H NMR (400 MHz, CDCl₃) δ ppm 8.68-8.28 (m, 2H) 6.23-5.53 (m, 1H) 4.44-3.32 (m, 5H) 1.97-1.73 (m, 2H) 1.52-1.34 (m, 9H) 1.06-0.94 (m, 2H) 0.80-0.64 (m, 1H) 0.27-0.00 (m, 1H).

### Description D33

### N-[(1R,4S,6R)-3-azabicyclo[4.1.0]hept-4-ylmethyl]-5-(trifluoromethyl)-2-pyrimidinamine (D33)

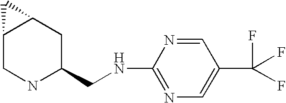

To a solution of 1,1-dimethylethyl (1R,4S,6R)-4-({[5-(trifluoromethyl)-2-pyrimidinyl]amino}methyl)-3-azabicyclo[4.1.0]heptane-3-carboxylate D32 (45 mg) in DCM (4 ml) TFA (2 ml, 26.0 mmol) was added dropwise. The mixture was left reacting at room temperature for 1 hour. Solvent was evaporated in vacuum and the crude was purified by SCX chromatography. It was recovered the title compound D33 (31 mg). UPLC: (Acid Final_QC): rt=0.46, peaks observed: 273 (M+1). C₁₂F₁₅F₃N₄ requires 272. ¹H NMR (400 MHz, CDCl₃) δ ppm 8.46 (d, 2H) 6.32 (br. s., 1H) 3.61-3.55 (m, 1H) 3.52-3.46 (m, 1H) 3.22-3.16 (m, 1H) 2.78-2.74 (m, 1H) 2.50-2.45 (m, 1H) 1.94-1.86 (m, 1H) 1.60-1.53 (m, 1H) 1.11-1.04 (m, 2H) 0.73-0.68 (m, 1H) 0.23-0.19 (m, 1H).

### Description 34

### [6-methyl-3-(propyloxy)-2-pyridinyl]methanol (D34)

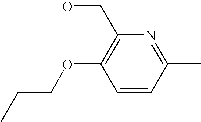

In a 250 ml round-bottom flask 2-(hydroxymethyl)-6-methyl-3-pyridinol (3 g, 21.56 mmol), 1-iodopropane (2.10 ml, 21.56 mmol) and potassium carbonate (14.90 g, 108 mmol) were dissolved in DMF (30 ml) and the mixture left under stirring overnight at room temperature. H₂O and EtOAc were added and the two layers were separated. The aqueous one was back-extracted several times with EtOAc. The combined organic phases were washed with brine/ice, dried (Na₂SO₄), filtered and concentrated under reduced pressure to give a crude material containing the title compound and some residual DMF. The residue was taken-up in water/ice and extracted with EtOAc. The organic phase was dried (Na₂SO₄) and concentrated under reduced pressure to afford the title compound D34 (3.60 g), which was used in the next step without any further purification. MS: (ES/+) m/z: 182 (M+1). C₁₀H₁₅NO₂ requires 181. ¹H-NMR (400 MHz, CDCl₃) δ (ppm): 6.95-7.09 (m, 2H), 4.73 (s, 2H), 3.94 (t, 2H), 2.50 (s, 3H), 1.75-1.91 (m, 2H), 1.05 (t, 3H).

### Description 35

### 6-methyl-3-(propyloxy)-2-pyridinecarboxylic acid (D35)

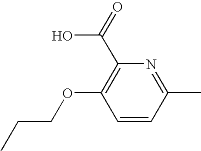

In a 500 ml round-bottom flask [6-methyl-3-(propyloxy)-2-pyridinyl]methanol D34 (3.50 g) was suspended in water (16 ml) and KMnO₄ (6.10 g, 38.60 mmol) and KOH (1 M aqueous solution, 19 ml, 19 mmol) were added. The mixture was stirred at room temperature for 2 hours. The pH was adjusted to 4 by addition of a 1 M aqueous HCl solution and then MeOH (100 ml) was added. The solid was filtered off, volatiles were removed under reduced pressure and the aqueous phase was extracted twice with DCM. The collected organic layers were washed with brine, dried (Na₂SO₄) and concentrated under reduced pressure to afford the title compound D35 (2 g). MS: (ES/+) m/z: 196 (M+1). C₁₀H₁₃NO₂ requires 195. ¹H-NMR (400 MHz, DMSO-d₆) δ (ppm): 12.96 (bs, 1H), 7.49 (d, 1H), 7.31 (d, 1H), 3.98 (t, 2H), 2.40 (s, 3H), 1.60-1.80 (m, 2H), 0.96 (t, 3H).

### Description 36

### [6-methyl-3-(methyloxy)-2-pyridinyl]methanol (D36)

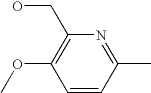

2-(hydroxymethyl)-6-methyl-3-pyridinol (2.10 g, 15.09 mmol), iodomethane (2.83 ml, 45.30 mmol) and potassium carbonate (10.43 g, 75 mmol) were dissolved in DMF (15 ml) and the mixture left under stirring at room temperature for 1 hour. Brine and EtOAc were added and the two layers were separated. The aqueous one was back-extracted several times with EtOAc. The combined organic phases were dried (Na₂SO₄), filtered through a phase separator tube and concentrated under reduced pressure to give the crude title compound D36 (2.30 g) which was used in the next step without any further purification. MS: (ES/+) m/z: 154 (M+1). C₈H₁₁NO₂ requires 153.

### Description 37

### 6-methyl-3-(methyloxy)-2-pyridinecarboxylic acid (D37)

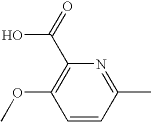

[6-methyl-3-(methyloxy)-2-pyridinyl]methanol D36 (0.10 g, of the crude material obtained in the Description 36) was suspended in water (7 ml) and KMnO₄ (0.21 g, 1.31 mmol) and KOH (1 M aqueous solution, 1 ml, 5 mmol) were added. The mixture was stirred at room temperature for 1.5 hours. The pH was adjusted to between 4 and 6 by addition of a 1 M aqueous HCl solution and the mixture was extracted several times with DCM. The collected organic layers were dried (Na₂SO₄), filtered through a phase separator tube and concentrated under reduced pressure to afford the title compound D37 (0.045 g). MS: (ES/+) m/z: 168 (M+1). C₈H₉NO₃ requires 167. ¹H-NMR (400 MHz, CDCl₃) δ (ppm): 7.43 (m, 2H), 4.00 (s, 3H), 2.55 (s, 3H).

### Description 38

### [3-(ethyloxy)-6-methyl-2-pyridinyl]methanol (D38)

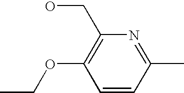

2-(hydroxymethyl)-6-methyl-3-pyridinol (1.50 g, 10.78 mmol), iodoethane (1.72 ml, 21.56 mmol) and potassium carbonate (7.45 g, 53.90 mmol) were dissolved in DMF (15 ml) and the mixture left under stirring at room temperature overnight. Water and EtOAc were added and the two layers were separated. The aqueous one was back-extracted several times with EtOAc. The combined organic phases were washed with brine/ice, dried (Na₂SO₄), filtered and concentrated under reduced pressure to afford the crude title compound D38 (1.67 g) as a pale yellow solid which was used in the next step without any further purification. MS: (ES/+) m/z: 168 (M+1). C₉H₁₃NO₂ requires 167. ¹H-NMR (400 MHz, CDCl₃) δ (ppm): 6.96-7.07 (m, 2H), 4.71 (s, 2H), 4.04 (q, 2H), 2.50 (s, 3H), 1.43 (t, 3H).

### Description 39

### 3-(ethyloxy)-6-methyl-2-pyridinecarboxylic acid (D39)

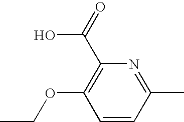

To a solution of [3-(ethyloxy)-6-methyl-2-pyridinyl]methanol D38 (1.67 g of the crude material obtained in the Description 38) in acetonitrile (50 ml) and phosphate buffer (38 ml), TEMPO (0.22 g, 1.40 mmol) was added and the mixture was heated to 35° C. NaClO₂ (4.51 g, 49.90 mmol) in water (10 ml) and NaClO (13 wt % aqueous solution, 18.96 ml, 39.90 mmol) were added simultaneously over 1 hour. The resulting reaction mixture was stirred at 35° C. for 4 hours, water (40 ml) was added and the pH was adjusted to 8 by addition of a 1 M aqueous NaOH solution. The mixture was poured into an ice-cooled aqueous saturated sodium thiosulfate solution (100 ml) and stirred for a further 30 minutes. The pH was adjusted to 3 by addition of a 1 M aqueous HCl solution and the aqueous phase was extracted with DCM (6×200 ml). The combined organic layers were washed with brine (2×200 ml), dried (Na₂SO₄) and concentrated under reduced pressure to afford the title compound D39 (1.64 g). MS: (ES/+) m/z: 182 (M+1). C₉H₁₁NO₃ requires 181. ¹H-NMR (400 MHz, DMSO-d₆) δ (ppm): 12.50-13.26 (bs., 1H), 7.49 (d, 1H), 7.31 (d, 1H), 4.08 (q, 2H), 2.40 (s, 3H), 1.29 (t, 3H).

### Description 40

### 2-methylfuro[3,4-b]pyridine-5,7-dione (D40)

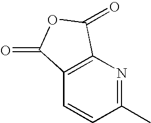

In a 100 ml round-bottomed flask 6-methyl-2,3-pyridinedicarboxylic acid (10 g, 55.2 mmol) and acetic anhydride (26 ml, 276 mmol) were added and heated at 100° C. under nitrogen for 5 hours. After this time the volatiles were removed under vacuum to give the title compound D40 (8.2 g) as a slightly brown solid. ¹H NMR (400 MHz, DMSO-d₆) δ ppm 8.41 (d, 1H), 7.82 (d, 1H), 2.73 (s, 3H).

### Description 41

### 6-methyl-2-[(methyloxy)carbonyl]-3-pyridinecarboxylic acid (D41)

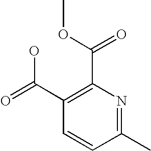

2-methylfuro[3,4-b]pyridine-5,7-dione D40 (3 g) was added portionwise over 5 minutes to stirred MeOH (20 ml) at 0° C. The mixture was stirred at 0° C. for 30 minutes then at room temperature for other 2.5 hours. The solution was evaporated at reduced pressure and the residue recrystallized from toluene (50 ml). The solid was filtered and dried under high vacuum for 30 minutes, obtaining a first batch of the title compound D41 (1.16 g) as pale brown solid. From the toluene solution new solid precipitated: this solid was filtered and dried under high vacuum for 30 minutes, obtaining a second batch of the title compound D41 (352 mg) as pale yellow solid. The toluene solution was then evaporated at reduced pressure and the residue recrystallized again from toluene (25 ml). The solid was filtered and dried under high vacuum for 30 minutes, obtaining a third batch of the title compound D41 (615 mg) as pale yellow solid. UPLC (Basic GEN_C): rt=0.23 minutes, peak observed: 195 (M+1). C₉H₉NO₄ requires 196. ¹H NMR (400 MHz, DMSO-d₆) δ ppm 13.61 (br. s., 1H), 8.09-8.31 (m, 1H), 7.51 (m, 1H), 3.82 (s, 3H), 2.55 (s, 3H).

### Description 42

### methyl 3-({[(1,1-dimethylethyl)oxy]carbonyl}amino)-6-methyl-2-pyridinecarboxylate (D42)

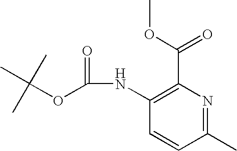

6-methyl-2-[(methyloxy)carbonyl]-3-pyridinecarboxylic acid D41 (1.15 g) was suspended in toluene (40 ml) and DIPEA (1.25 ml, 7.16 mmol) was added, causing the complete dissolution of the solid. This mixture was stirred 10 minutes at room temperature, then diphenyl azidophosphate (1.35 ml, 6.26 mmol) was added in one portion and the mixture was stirred at reflux for 1 hour. The solution was cooled at room temperature and t-BuOH (2.5 ml, 26 mmol) was added in one portion. The mixture was then stirred at 70° C. for 1 hour and then cooled at room temperature, Et₂O (50 ml) was added and the resulting solution washed with NaHCO₃ saturated solution (3×60 mls). The water phases were joined together and back-extracted with Et₂O (50 mls). The two organic solutions were joined together, dried over Na₂SO₄ and evaporated at reduced pressure, obtaining the crude target material as pale yellow oil. This material was purified by flash chromatography on silica gel (Biotage, EtOAc/Cy from 10/90 to 70/30; Snap-100 g column). The title compound D42 (1.315 g) was obtained as white solid. UPLC (Basic GEN_C): rt=0.68 minutes, peak observed: 267 (M+1). C₁₃H₁₈N₂O₄ requires 266. ¹H NMR (400 MHz, CDCl₃) δ ppm 10.13 (bs., 1H), 8.77 (d, 1H), 7.34 (d, 1H), 4.03 (s, 3H), 2.59 (s, 3H), 1.53-1.56 (m, 9H).

### Description 43

### methyl 3-amino-6-methyl-2-pyridinecarboxylate (D43)

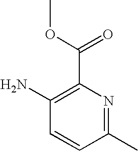

Methyl 3-({[(1,1-dimethylethyl)oxy]carbonyl}amino)-6-methyl-2-pyridinecarboxylate D42 (1.3 g) was dissolved in DCM (80 ml) and the mixture stirred at 0° C. A solution of TFA (5 ml, 64.9 mmol) in DCM (10 ml) was dropped into the cold mixture over 3 minutes. The resulting solution was left under stirring at 0° C. for 30 minutes, then the mixture was left still at room temperature overnight. TFA (4 ml, 51.9 mmol) dissolved in DCM (10 ml) was added over 3 minutes and the mixture stirred again at room temperature for 5 hours. The solution was loaded onto an SCX-25 g column and the column was eluted firstly with DCM (100 mls) and then MeOH (20 mls). The material was collected eluting with NH₃ (2M in MeOH, 100 mls) and after evaporation under reduced pressure of the ammonia solution it was obtained the title compound D43 (770 mg) was obtained as a white solid. UPLC (Basic GEN_C): rt=0.44 minutes, peak observed: 167 (M+1). C₈H₁₀N₂O₂ requires 166. ¹H NMR (400 MHz, CDCl₃) δ ppm 7.14 (d, 1H), 7.01 (d, 1H), 3.99 (s, 3H), 2.52 (s, 3H).

### Description 44

### methyl 3-iodo-6-methyl-2-pyridinecarboxylate (D44)

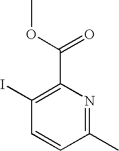

HCl 6 M solution in water (4.5 ml, 27.0 mmol) was added to methyl 3-amino-6-methyl-2-pyridinecarboxylate D43 (768 mg) and the resulting pale yellow mixture was sequentially diluted with water (4×5 ml) and chilled at 0° C. (internal temperature).

A solution of sodium nitrite (480 mg, 6.96 mmol) in water (2 ml) was dropped into the mixture over 1 minute. After this addition the mixture was stirred at 0° C. for 30 minutes, then a solution of KI (1.69 g, 10.18 mmol) in water (2 ml) was added over 1 minute, causing the formation of a dark violet crust (moderate gas evolution). The mixture was left under stirring for 1 hour: during this period the temperature passed from 0° C. to +5° C. EtOAc (50 ml) was then added to the stirred mixture, causing the dissolution of the dark solid. Water (50 ml) and EtOAc (50 ml) were added and the whole mixture was poured into a separator funnel. After the separation of the two phases, the water phase was extracted with EtOAc. All the organic phases were joined together and washed with NaHCO₃ saturated solution; the acidic water phase was neutralized by the addition of the previously used NaHCO₃ saturated solution and the resulting mixture extracted with EtOAc (2×50 mls). All the organic phases were joined together, dried over Na₂SO₄ and evaporated at reduced pressure, obtaining the crude target material as dark brown/violet oil. This material was purified by silica gel chromatography (Biotage SP4 Snap-100 g column, EtOAc/Cy from 10/90 to 30/70). The title compound D44 was obtained as a pale brown solid (1.1 g). UPLC (Basic GEN_C): rt=0.68 minutes, peak observed: 278 (M+1). C₈H₈INO₂ requires 277. ¹H NMR (400 MHz, CDCl₃) δ ppm 8.12 (d, 1H), 7.01 (d, 1H), 4.01 (s, 3H), 2.58 (s, 3H).

### Description 45

### methyl 6-methyl-3-(2-pyrimidinyl)-2-pyridinecarboxylate (D45)

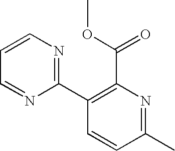

To a suspension of methyl 3-iodo-6-methyl-2-pyridinecarboxylate D44 (300 mg), CsF (329 mg, 2.166 mmol) and Pd(Ph₃P)₄ (50.0 mg, 0.043 mmol) in DMF (10 ml) stirred under nitrogen at room temperature was added 2-(tributylstannanyl)pyrimidine (480 mg, 1.299 mmol). The reaction mixture was stirred at 130° C. for 30 minutes at microwave Personal Chemistry. The reaction mixture was partitioned between EtOAc and aqueous NaHCO₃ saturated solution the combined organic phases were dried to give the crude product which was purified by silica gel chromatography (SNAP KP-NH 55 g; Cy/EtOAc 15 column volumes from 100/0 to 70/30). Collected fractions were evaporated to obtain the title compound D45 (101 mg) as white solid. UPLC (Basic GEN_C): rt=0.56 minutes, peak observed: 230 (M+1). C₁₂H₁₁N₃O₂ requires 229. ¹H NMR (400 MHz, DMSO-d₆) δ ppm 8.92 (d, 2H), 8.49 (d, 1H), 7.44-7.63 (m, 2H), 3.75 (s, 3H), 2.57 (s, 3H).

### Description 46

### 6-methyl-3-(2-pyrimidinyl)-2-pyridinecarboxylic acid lithium salt (D46)

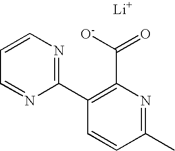

To a solution of methyl 6-methyl-3-(2-pyrimidinyl)-2-pyridinecarboxylate D45 (100 mg) in MeOH (4.5 ml) and water (1.1 ml) was added LiOH (13.58 mg, 0.567 mmol) and the resulting mixture was submitted to microwave irradiation at 60° C. for 85 minutes. After this time the solvents were removed under reduced pressure to give the title compound D46 (100 mg) as a white solid. C₁₁H₈N₃O₂.Li⁺ requires 221. ¹H NMR (400 MHz, DMSO-d₆) δ ppm 8.78 (m, 2H), 7.86 (m, 1H), 7.37 (m, 1H), 7.24 (m, 1H), 2.50 (s, 3H).

### Description 47

### methyl 6-methyl-3-(4-methyl-1,3-thiazol-2-yl)-2-pyridinecarboxylate (D47)

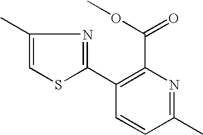

4-methyl-2-(tributylstannanyl)-1,3-thiazole (150 mg, 0.386 mmol) was dissolved in 1,4-Dioxane (2.5 ml). To the stirred solution methyl 3-iodo-6-methyl-2-pyridinecarboxylate D44 (100 mg) was added, followed by Pd(Ph₃P)₄ (41.7 mg, 0.036 mmol).

The resulting orange solution was heated into a microwave reactor at 120° C. for 30 minutes. The mixture was loaded onto an SCX-5 g column the column was eluted and after evaporation under reduced pressure of the solvent it was obtained the crude target material as colorless oil, which was then purified by flash chromatography on silica gel (Biotage SNAP-10 g silica gel column, EtOAc/Cy 25:75). It was obtained the title compound D47 as white solid (74 mg). UPLC (Acid GEN_C): rt=0.62 minutes, peak observed: 249 (M+1). C₁₂H₁₂N₂O₂S requires 248. ¹H NMR (400 MHz, CDCl₃) δ ppm 7.97 (d, 1H), 7.33 (d, 1H), 6.98 (s, 1H), 3.94 (s, 3H), 2.66 (s, 3H), 2.50 (s, 3H).

### Description 48

### 6-methyl-3-(4-methyl-1,3-thiazol-2-yl)-2-pyridinecarboxylate lithium salt (D48)

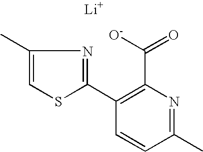

methyl 6-methyl-3-(4-methyl-1,3-thiazol-2-yl)-2-pyridinecarboxylate D47 (73 mg) was dissolved in EtOH (1 ml) into a capped vial, then a solution of LiOH (8.5 mg, 0.355 mmol) in water (0.5 ml) was added in one portion. The mixture was then stirred at room temperature for 3 hours. The solvent was evaporated at reduced pressure, obtaining the title compound D48 as pale yellow solid (73 mg). UPLC (Basic GEN_QC): rt=0.36 minutes, peak observed: 232 (M−1). C₁₁H₉N₂O₂S Li⁺ requires 233. ¹H NMR (400 MHz, DMSO-d₆) δ ppm 8.04 (d, 1H), 7.22 (d, 1H), 7.08 (d, 1H), 2.39 (s, 3H), 2.42 (s, 3H).

### Description 49

### methyl 6-methyl-3-(2H-1,2,3-triazol-2-yl)-2-pyridinecarboxylate (D49)

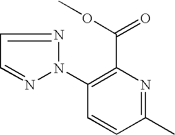

DMF (1.5 ml) was added to a mixture of methyl 3-iodo-6-methyl-2-pyridinecarboxylate D44 (100 mg), 1H-1,2,3-triazole (49.9 mg, 0.722 mmol), (1R,2R)—N,N′-dimethyl-1,2-cyclohexanediamine (10.27 mg, 0.072 mmol), CuI (3.44 mg, 0.018 mmol) and Cs₂CO₃ (235 mg, 0.722 mmol) in a microwave vial. The mixture was degassed via three vacuum/nitrogen cycles then irradiated in a single mode microwave reactor to 120° C. for 20 minutes. The mixture was irradiated in a single mode microwave reactor to 120° C. for a further 40 minutes. The reaction mixture was cooled and filtered washing the solids with EtOAc (20 mls). The solids were dissolved in ph=3 buffer solution (5 ml); UPLC check of this aqueous solution showed that it contained a considerable quantity of 6-methyl-3-(2H-1,2,3-triazol-2-yl)-2-pyridinecarboxylic acid. The aqueous phase was extracted repeatedly with DCM; the combined DCM extracts were diluted with MeOH (50 ml) and treated with TMS-diazomethane. The volatiles were evaporated to give a yellow residue that was purified by flash chromatography on silica gel (Biotage, SNAP 10 g column, 10%-50% EtOAc/Cy) to give the title compound D49 (38 mg) as a white solid. UPLC (Basic QC_POS_{—}50-800): rt=0.57 minutes, peak observed: 219 (M+1). C₁₀H₁₀N₄O₂ requires 218. ¹H NMR (400 MHz, CDCl₃) δ ppm 8.20 (d, 1H), 7.87 (s, 2H), 7.44 (d, 1H), 3.94 (s, 3H), 2.71 (s, 3H).

### Description 50

### 6-methyl-3-(2H-1,2,3-triazol-2-yl)-2-pyridinecarboxylic acid (D50)

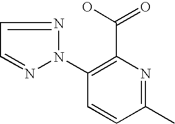

A solution of methyl 6-methyl-3-(2H-1,2,3-triazol-2-yl)-2-pyridinecarboxylate D49 (36 mg) and LiOH (5.93 mg, 0.247 mmol) in THF/water (2:1, 3 ml) was stirred overnight. The mixture was evaporated under reduced pressure; the residue was taken up in water (2 ml) and neutralised with 1 M HCl water solution and then loaded onto a pre-conditioned C18 5 g column (the column was eluted with water and then MeOH). The methanol fractions were evaporated under reduced pressure to give the title compound D50 (34 mg) as a white solid. UPLC (Basic QC_POS_{—}50-800): rt=0.30 minutes. peak observed: 205 (M+1). C₉H₈N₄O₂ requires 204. ¹H NMR (400 MHz, MeOH) δ (ppm) 8.24 (d, 1H), 7.99 (s, 2H), 7.61 (d, 1H), 2.67 (s, 3H).

### Description 51

### methyl 3-(4-fluorophenyl)-6-methylpyridine-2-carboxylate (D51)

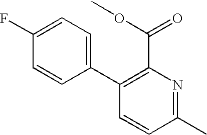

Methyl 3-iodo-6-methyl-2-pyridinecarboxylate D44 (40 mg) and (4-fluorophenyl)boronic acid (Aldrich, 40.4 mg, 0.289 mmol) were suspended in 1 ml of EtOH and 1 ml of toluene. Pd(Ph₃P)₄ (16.68 mg, 0.014 mmol) and Na₂CO₃ (0.361 ml, 0.722 mmol) were then added. The reaction was shaken at 90° C. for 3 hours.

Volatiles were removed under vacuum and the residue was purified by silica gel chromatography (Biotage SP, Column size SNAP 25 g, using a gradient starting from Cy:EtOAc 8:2 to EtOAc 100%) to give the title compound D51(32 mg) as a white solid. UPLC (Basic GEN_QC): rt=0.77 minutes. peak observed: 246 (M+1). C₁₄H₁₂FNO₂ requires 245. ¹H NMR (400 MHz, DMSO-d₆) δ (ppm) 7.84 (d, 1H), 7.51 (d, 1H), 7.42-7.35 (m, 2H), 7.34-7.27 (m, 2H), 3.65 (s, 3H), 2.55 (s, 3H).

### Description 52

### 3-(4-fluorophenyl)-6-methyl-2-pyridinecarboxylate Lithium salt (D52)

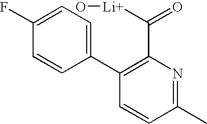

Methyl 3-(4-fluorophenyl)-6-methyl-2-pyridinecarboxylate D51 (30 mg) was dissolved in EtOH (1 ml) and water (1 ml) then LiOH (4.39 mg, 0.183 mmol) was added and the reaction was stirred at room temperature overnight. All volatiles were removed under vacuum using Biotage V10 system to give the title compound D52 (39 mg). The compound was used without any further purification. C₁₃H₉FNO₂.Li⁺ requires 237. ¹H NMR (400 MHz, DMSO-d6) δ (ppm) 7.8-7.5 (m, 3H), 7.2-7.05 (m, 3H), 2.42 (s, 3H).

### Description 53

### 2-chloro-N-(2-hydroxybutyl)-6-methyl-3-pyridinecarboxamide (D53)

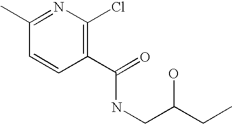

2-chloro-6-methyl-3-pyridinecarboxylic acid (2.5 g, 14.57 mmol) (available from Sigma-Aldrich #357847) was dissolved in DMF (35 ml) and DIPEA (7.63 ml, 43.7 mmol) was added. To this mixture TBTU (5.15 g, 16.03 mmol) was added in one portion and the resulting orange solution was stirred 45 minutes at room temperature. 1-amino-2-butanol (2.5 g, 28.0 mmol) was then added dissolved in DMF (5 ml) and the resulting mixture stirred at room temperature for 90 minutes. The mixture was then stored into the fridge over the weekend. The mixture was partitioned between NaHCO₃ saturated solution and Et₂O; the water layer was extracted with Et₂O. The water layer was then extracted with EtOAc. The organic phases deriving from the Et₂O extractions were joined and dried over Na₂SO₄ and evaporated at reduced pressure; the oily residue was dried under high vacuum at 45° C. for 2 hours, obtaining a first batch of crude material purified by flash chromatography on silica gel (Biotage 100 g column, EtOAc/Cy from 30:70 to 75:25). The organic phases deriving from the EtOAc extractions were joined and dried over Na₂SO₄ and evaporated at reduced pressure; the oily residue was dried under high vacuum at 45° C. for 1 hour, obtaining a second batch of crude material, purified by flash chromatography on silica gel (Biotage 340 g column, EtOAc/Cy from 30:70 to 75:25). The fractions eluted performing the two purifications were joined together and then evaporated at reduced pressure it was obtained the title compound D53 as pale yellow oil (3.62 g). UPLC (Basic GEN_QC): rt=0.45 minutes, peaks observed: 243 (M+1). C₁₁H₁₅ClN₂O₂ requires 242. ¹H NMR (400 MHz, DMSO-d₆) δ ppm 8.45 (m, 1H), 7.77 (m, 1H), 7.33 (m, 1H), 4.69 (m, 1H), 3.43-3.61 (m, 1H), 3.05-3.30 (m, 2H), 2.48 (s, 3H), 1.51 (m, 1H), 1.18-1.42 (m, 1H), 0.90 (t, 3H).

### Description 54

### 2-chloro-6-methyl-N-(2-oxobutyl)-3-pyridinecarboxamide (D54)

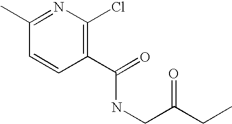

2-chloro-N-(2-hydroxybutyl)-6-methyl-3-pyridinecarboxamide D53 (3.62 g) was dissolved in DCM (100 ml), then, to the stirred solution, Dess-Martin periodinane (6.75 g, 15.91 mmol) was added portionwise over 5 minutes. The mixture was stirred at room temperature for 45 minutes (white suspension). The mixture was then partitioned between NaHCO₃ saturated solution and DCM; water layer extracted with DCM. The organic phases were joined, dried over Na₂SO₄ and evaporated at reduced pressure, obtaining the crude target material as pale yellow solid (7.2 g). This material was stored in the fridge overnight and was purified by flash chromatography on silica gel (Snap-340 g column, EtOAc/Cy from 20:80 to 80:20) to give the title compound D54 (3.11 g) as white solid. UPLC (Basic GEN_C): rt=0.50 minutes. peaks observed: 241 (M+1). C₁₁H₁₃ClN₂O₂ requires 240. ¹H NMR (400 MHz, DMSO-d₆) δ ppm 8.82 (m, 1H), 7.81 (m, 1H), 7.37 (m, 1H), 4.09 (d, 2H), 3.30-3.35 (s, 3H), 2.53-2.59 (m, 2H), 0.97 (t, 3H).

### Description 55

### 2-chloro-3-(5-ethyl-1,3-oxazol-2-yl)-6-methylpyridine (D55)

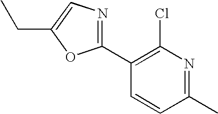

2-Chloro-6-methyl-N-(2-oxobutyl)-3-pyridinecarboxamide D54 (3.051 g) was dissolved in THF (100 ml) and Burgess reagent (3.104 g, 13.03 mmol) was added in one portion. The pale yellow solution was stirred at room temperature for 4.5 hours, then new Burgess reagent (0.41 g, 1.72 mmol) was added and the mixture stirred at 60° C. for 1.5 hours, the solvent was evaporated at reduced pressure and the residue partitioned between NaHCO₃ saturated solution and EtOAc; water layer was extracted with EtOAc. The organic phases were joined and dried over Na₂SO₄ and evaporated at reduced pressure, obtaining the crude target material, which was then purified by flash chromatography on silica gel (Snap-100 g column, EtOAc/Cy from 20:80 to 90:10). After evaporation at reduced pressure it was obtained the title compound D55 (1.7 g) colourless oil, which slowly solidified upon standing at room temperature and the unreacted starting material. UPLC (Basic GEN_C): rt=0.77 minutes. peaks observed: 223 (M+1). C₁₁H₁₁ClN₂O requires 222. ¹H NMR (400 MHz, CDCl₃) δ ppm 8.21 (d, 1H), 7.21 (d, 1H), 6.96 (s, 1H), 2.80 (m, 2H), 2.62 (s, 3H), 1.35 (t, 3H).

### Description 56

### 2-ethenyl-3-(5-ethyl-1,3-oxazol-2-yl)-6-methylpyridine (D56)

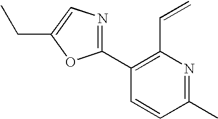

2-chloro-3-(5-ethyl-1,3-oxazol-2-yl)-6-methylpyridine D55 (168 mg), Pd(Ph₃P)₄ (70 mg, 0.061 mmol), 2-ethenyl-4,4,5,5-tetramethyl-1,3,2-dioxaborolane (0.2 ml, 1.179 mmol) and K₂CO₃ (209 mg, 1.509 mmol) were mixed together, then 1,4-dioxane (8 ml) and water (3 ml) were added. The mixture was stirred at 80° C. for 30 minutes. The mixture was stirred again at 80° C. for other 50 minutes. The solvents were evaporated at reduced pressure and the residue partitioned between NaHCO₃ saturated solution and Et₂O; water layer extracted with Et₂O. The organic phases were joined and dried over Na₂SO₄ and evaporated at reduced pressure, obtaining the crude target material which was purified by flash chromatography on silica gel (Snap-25 g column, EtOAc/Cy from 5:95 to 30:70). It was obtained the title compound D56 as white solid (135 mg). UPLC (Basic GEN_C): rt=0.88 minutes, peaks observed: 215 (M+1). C₁₃H₁₄N₂O requires 214. ¹H NMR (400 MHz, CDCl₃) δ ppm 8.10 (m, 1H), 7.87 (m, 1H), 7.15 (m 1H), 6.92 (s, 1H), 6.56 (m, 1H), 5.61 (m, 1H), 2.68-2.87 (m, 2H), 2.63 (s, 3H), 1.34 (t, 3H).

### Description 57

### 3-(5-ethyl-1,3-oxazol-2-yl)-6-methyl-2-pyridinecarbaldehyde (D57)

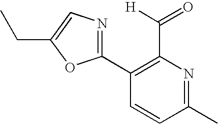

2-Ethenyl-3-(5-ethyl-1,3-oxazol-2-yl)-6-methylpyridine D56 (132 mg) was dissolved in THF (3 ml) and water (3 ml). To this stirred mixture a solution of OsO₄ 4% in water (0.390 ml, 0.050 mmol) was added over 30 seconds and the resulting mixture was then stirred at room temperature for 5 minutes. Sodium periodate (329 mg, 1.538 mmol) was then added in one portion and the resulting mixture was left to stir at room temperature for 70 minutes. The mixture was then partitioned between NaHCO₃ saturated solution and Et₂O; water layer extracted with Et₂O. The organic phases were joined and dried over Na₂SO₄ and evaporated at reduced pressure, obtaining the title compound D57 as brown solid (136 mg). UPLC (Basic GEN_C): rt=0.65 minutes, peaks observed: 217 (M+1). C₁₂H₁₂N₂O₂ requires 216. ¹H NMR (400 MHz, CDCl₃) δ ppm 10.75 (s, 1H), 8.25 (d, 1H), 7.45 (d, 1H), 6.98 (s, 1H), 2.76-2.91 (m, 2H), 2.74 (s, 3H), 1.35 (t, 3H).

### Description 58

### 3-(5-ethyl-1,3-oxazol-2-yl)-6-methyl-2-pyridinecarboxylic acid (D58)

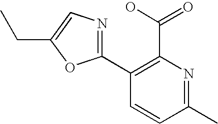

3-(5-ethyl-1,3-oxazol-2-yl)-6-methyl-2-pyridinecarbaldehyde D57 (550 mg) was dissolved in DMSO (5 ml) and citric pH=3 buffer solution (1.5 ml) and the mixture was chilled at 0° C. NaClO₂ 1 M in water (7 ml, 7.00 mmol) was dropped into the mixture over 10 minutes, then the stirring was continued at room temperature. New citric pH=3 buffer solution (1.5 ml), followed by new NaClO₂ 1 M in water (3 ml, 3.00 mmol) were dropped into the mixture, which was then stirred at room temperature for other 30 minutes, then the whole mixture has been stored in the fridge overnight. NaClO₂ 1 M in water (1 ml, 3.00 mmol) was dropped into the mixture, which was then stirred at room temperature for other 30 minutes. The whole dark mixture has been loaded onto a C18-70 g column (eluted with water then with MeOH). After evaporation at reduced pressure of the methanol fractions it was obtained the crude dark brown oil, which solidified by Et₂O (2 ml) addition. To this solid acetone (2.5 ml) and Et₂O (3 ml) were added. The solid was filtered and dried under high vacuum for 30 minutes, giving the dark brown solid (23 mg). To the solution Et₂O (8 ml) was added and the so obtained mixture was stored for 70 minutes into the fridge. This solid was filtered and washed with Et₂O (3 ml). All the organic solution (mother organic solution and Et₂O of washing) were joined, evaporated at reduced pressure and dried under high vacuum at 45° C. for 30 minutes, giving the title compound D58 as brown gum (362 mg). UPLC (Basic GEN_C): rt=0.35 minutes, peaks observed: 231 (M−1). C₁₂H₁₂N₂O₃ requires 232. ¹H NMR (400 MHz, DMSO-d₆) δ ppm 8.20 (d, 1H), 7.50 (d, 1H), 7.05 (s, 1H), 2.61-2.82 (m, 3H), 2.55 (s, 3H), 1.23 (m, 3H).

### Description 59

### methyl 2-chloro-6-methyl-3-pyridinecarboxylate (D59)

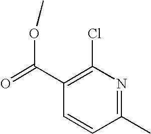

To a solution of 2-chloro-6-methyl-3-pyridinecarboxylic acid (8 g, 46.6 mmol) in DCM (100 ml) and MeOH (50.0 ml) stirred under nitrogen at room temperature was added TMS-diazomethane 2 M in hexane (46.6 ml, 93 mmol). The reaction mixture was stirred at room temperature for 20 minutes. The solvents were removed to give the title compound D59 (7 g). MS: (ES/+) m/z: 186 (M+1). C₈H₈ClNO₂ requires 185. ¹H NMR (400 MHz, CDCl₃) δ ppm 8.10 (d, 1H), 7.18 (d, 1H), 3.96 (s, 3H), 2.61 (s, 3H).

### Description 60

### methyl 2-bromo-6-methyl-3-pyridinecarboxylate (D60)

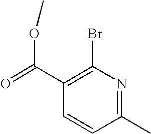

To a stirred solution of methyl 2-chloro-6-methyl-3-pyridinecarboxylate D59 (500 mg) in propionitrile (2 ml) under nitrogen at room temperature bromotrimethylsilane (0.699 ml, 5.39 mmol) was added dropwise neat. The reaction mixture was heated at Microwave Personal Chemistry 20 min at 160° C. The solvent was removed to give the crude. Under similar conditions another batch of D59 (500 mg) was processed to give the crude title compound. The two crudes were joined and purified together by flash chromatography on silica gel (80 g column, Cy 100% to Cy/EtOAc 4:6) to give the title compound D60 (1.2 g). ¹H NMR (400 MHz, CDCl₃) δ ppm 8.02 (d, 1H), 7.20 (d, 1H), 3.96 (s, 3H), 2.62 (s, 3H)

### Description 61

### methyl 2-ethenyl-6-methyl-3-pyridinecarboxylate (D61)

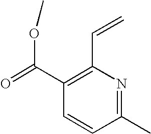

To a solution of methyl 2-bromo-6-methyl-3-pyridinecarboxylate D60 (1.15 g) and Pd(Ph₃P)₄ (0.2 g, 0.173 mmol) in 1,4-Dioxane (10 ml) stirred under nitrogen at room temperature tributyl(ethenyl)stannane (1.74 g, 5.50 mmol) was added neat in one charge. The reaction mixture was stirred at microwave Personal Chemistry at 95° C. for 30 minutes. The solvent was removed to give the crude title compound. Under similar conditions another batch of D60 (100 mg) was processed to give the crude title compound. The two crudes were joined and purified by flash chromatography on silica (80 g column, gradient elution from Cy to Cy/EtOAc 4:6) to afford the title compound D61 (1.0 g). UPLC (Basic GEN_C): rt=0.73 minutes, peak observed: 178 (M+1). C₁₀H₁₁NO₂ requires 177. ¹H NMR (400 MHz, CDCl₃) δ ppm 8.08 (d, 1H), 7.66 (dd, 1H), 7.12 (d, 1H), 6.52 (d, 1H), 5.59 (dd, 1H), 3.93 (s, 3H), 2.63 (s, 3H).

### Description 62

### 2-ethenyl-6-methyl-3-(3-methyl-1,2,4-oxadiazol-5-yl)pyridine (D62)

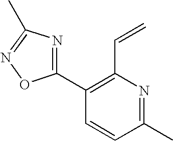

To a suspension of NaH 60% oil dispersion (0.903 g, 22.57 mmol) and molecular sieves 4 Å in dry THF (10 ml) stirred under nitrogen at room temperature acetamide oxime (0.836 g, 11.29 mmol) was added and the reaction stirred at room temperature for 30 minutes then a solution of methyl 2-ethenyl-6-methyl-3-pyridinecarboxylate D61 (1 g) in dry THF 10 ml was added in one charge. The reaction mixture was heated at the microwave Personal Chemistry at 100° C. for 30 minutes. NaHCO₃ saturated aqueous solution was added and the aqueous extracted with EtOAc, the organic passed through a hydrophobic frit, the solvent removed to give the crude product which was purified by flash chromatography on silica (80 g column, gradient elution from Cy to Cy/EtOAc 40/60) to afford the title compound D62 (308 mg). UPLC (Basic GEN_C): rt=0.78 minutes. Peak observed: 202 (M+1). C₁₁H₁₁N₃O requires 201. ¹H NMR (400 MHz, CDCl₃) δ ppm 8.21 (d, 1H), 7.83 (m, 1H), 7.22 (d, 1H), 6.65 (m, 1H), 5.69 (m, 1H), 2.67 (s, 3H), 2.52 (s, 3H).

### Description 63

### 6-methyl-3-(3-methyl-1,2,4-oxadiazol-5-yl)-2-pyridinecarbaldehyde (D63)

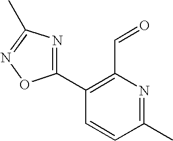

To a solution of 2-ethenyl-6-methyl-3-(3-methyl-1,2,4-oxadiazol-5-yl)pyridine D62 (100 mg) in THF (3 ml) and water (4.5 ml) stirred under nitrogen at room temperature was added a solution of OsO₄ 4% in water (0.39 ml, 0.05 mmol) and after 5 minutes in one charge sodium periodate (319 mg, 1.491 mmol). The reaction mixture was stirred at room temperature for 2 hours. The mixture was poured into a separatory funnel washed with brine and the aqueous extracted with EtOAc, the phases were separated on a hydrophobic frit, the combined organic solvent was removed to give the crude product which was purified by flash chromatography on silica gel (25 g column, gradient elution from Cy to Cy/EtOAc 80/20) to afford the title compound D63 (93 mg). UPLC (Basic GEN_C): rt 1=0.50 minutes, rt 2=0.55 minutes, peaks observed: 204 (M+1). C₁₀H₉N₃O₂ requires 203. ¹H NMR (400 MHz, CDCl₃) δ ppm 10.55 (s, 1H), 8.21 (m, 1H), 7.53 (m, 1H), 2.78 (s, 3H), 2.52-2.56 (m, 3H).

### Description 64

### 6-methyl-3-(3-methyl-1,2,4-oxadiazol-5-yl)-2-pyridinecarboxylic acid (D64A/D64B)

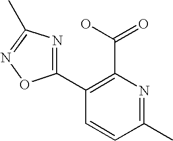

A) To a solution of 6-methyl-3-(3-methyl-1,2,4-oxadiazol-5-yl)-2-pyridinecarbaldehyde D63 (90 mg) in THF (3.00 ml) and water (6 ml) stirred at 0° C. was added solid NaOH (17.72 mg, 0.443 mmol) and after 10 minutes KMnO₄ (140 mg, 0.886 mmol) in one charge. The reaction mixture was stirred for 10 minutes. While still cold the reaction mixture was filtered on celite and the celite washed with HCl 1 M water solution and water. The aqueous filtrate at pH 1 was passed through a 50 g C18 column (MeOH, water to condition, water and then MeOH to elute) to afford the title compound D64A (70 mg). MS: (ES/−) m/z: 218 (M−1). C₁₀H₉N₃O₃ requires 219. ¹H NMR (400 MHz, CDCl₃) δ ppm 8.02 (d, 1H), 7.60 (d, 1H), 2.77 (s, 3H), 2.55 (s, 3H).

B) An alternative method to make D64 is: 6-methyl-3-(3-methyl-1,2,4-oxadiazol-5-yl)-2-pyridinecarbaldehyde D63 (0.89 mg) was dissolved in a mixture of DMSO (10 ml) and pH=3 buffer solution (3 ml) and the solution was cooled to 0° C. A 1 M solution of NaClO₂ in water (16 ml) was added; the solution turned to pale yellow and after the addition was left stirring at room temperature for 2 hours. New pH=3 buffer solution (1.5 ml) was added and the stirring was continued for 1 hour. The mixture was eluted through a 70 g C18 cartridge (preconditioned with MeOH and then with water; eluted with water and then with MeOH). The methanol fractions were joined and evaporated under reduced pressure to afford the title compound D64B (0.89 g).

### Description 65

### 6-methyl-3-(tributylstannanyl)-2-{[(tributylstannanyl)oxy]carbonyl}pyridine (D65)

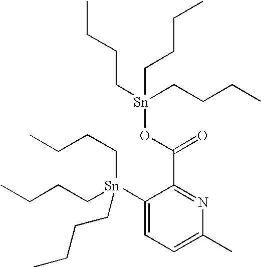

In a 100 ml double necked flask, anhydrous THF (4 ml) and 2,2,6,6-tetramethylpiperidine (0.372 ml, 2.188 mmol) were added and the resulting solution cooled to −78° C. To this solution sec-butyllithium (2.083 ml, 2.92 mmol) was added dropwise, over a period of 10 min. After stirring at −78° C. for additional 15 min a solution of 6-methyl-2-pyridinecarboxylic acid (100 mg, 0.729 mmol) in anhydrous THF (1 ml) was added, over 10 minutes. The resulting dark mixture was stirred at −78° C. for 10 min, then it was allowed to reach 0° C. and stirred at this temperature for 30 min. After this period a solution of tributyl(chloro)stannane (0.787 ml, 2.92 mmol) in THF (1 ml) was added to the reaction mixture at 0° C. and then warmed to room temperature and stirred for 1 hour.

The solvent was removed under reduced pressure and the orange residue obtained was filtered, the organic layer was concentrated to give the title compound D65 (1.05 g) in mixture with tributyl(chloro)stannane. It was used without further purification, yield supposed to be quantitative. UPLC (Acid GEN_QC): rt 1=1.03 minutes, peak observed: 426 [(M+1−Sn(Bu)₃] average. C₃₁H₅₉NO₂Sn₂ requires 715 average.

### Description 66

### 6-methyl-3-phenyl-2-pyridinecarboxylic acid (D66)

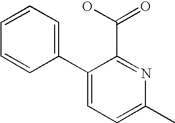

Triphenylphosphine (19.11 mg, 0.073 mmol) and bis(triphenylphosphine)palladium(II) chloride (25.6 mg, 0.036 mmol) were added to a solution of 6-methyl-3-(tributylstannanyl)-2-{[(tributylstannanyl)oxy]carbonyl}pyridine D65 (521 mg) in toluene (2.023 ml). The resulting mixture was refluxed for 1 hour and then it was cooled to room temperature, filtered over a celite pad washing with ethyl acetate and 2 M aqueous solution of NaOH. The aqueous layer was washed twice with EtOAc, acidified with 4 M aqueous solution of HCl and extracted with EtOAc. The collected organic layers were dried over Na₂SO₄, filtered and the evaporated under reduced pressure to give a title compound as solid which was triturated with hexane, the solid was filtered and dried to give the title compound D66 (60 mg) which was used without any further purification. C₁₃H₁₁NO₂ requires 213. ¹H NMR (400 MHz, DMSO-d6) δ (ppm) 7.81-7.75 (m, 1H), 7.48-7.42 (m, 3H), 7.42-7.37 (m, 3H), 2.53 (s, 3H).

### Description 67

### 3-(5,5-Dimethyl-1,3,2-dioxaborinan-2-yl)-6-methyl-2-pyridinecarbonitrile (D67)

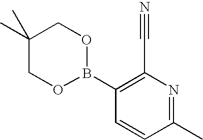

2,2,6,6-tetramethylpiperidine (3.49 ml, 20.52 mmol) was dissolved in dry THF (25 ml) under argon and stirred at −30° C.; BuLi (13.33 ml, 21.33 mmol) 1.6 M in hexane was added over 5 min (the temperature never exceeded −25° C.). The yellow solution was stirred at −30° C. for 20 min, then chilled at −78° C. and tris(1-methylethyl) borate (4.38 ml, 18.96 mmol) was added over 5 min (the temperature never exceeded −73° C.).

After 10 min at −78° C., 6-methyl-2-pyridinecarbonitrile (2.0 g, 16.93 mmol) dissolved in dry THF (14 ml) was added dropwise (over 20 min) maintaining internal temperature below −73° C. and the mixture became dark-brown. The mixture was stirred at −73° C. for 2 hours. The mixture was quenched with AcOH (2.374 ml, 41.5 mmol) dropwise at −73° C. (the temperature never exceeded −60° C. and the mixture became brilliant orange). The cooling bath was removed and the mixture left to reach the room temperature: during this period the mixture became thick and new THF (8 ml) had to be added in order to have a better stirring. The mixture was stirred 10 min at room temperature then 2,2-dimethyl-1,3-propanediol (2.409 g, 23.13 mmol) was added in one portion and the mixture stirred at room temperature overnight. The solvent was evaporated and the orange residue taken-up with DCM (100 ml) and 10% water solution of KH₂PO₄ (100 ml). The phases were separated and the water phase was back-extracted with DCM (50 ml). The combined organic phases were washed with 10% water solution of KH₂PO₄ (50 ml). The DCM was evaporated. The residue was dissolved in Et₂O (100 ml) and extracted with NaOH 0.05 M (5×50 ml, boronic ester in water phase). The aqueous phases were joined together and the pH was adjusted between pH=4 and pH=5 with 10% water solution of KH₂PO₄ (50 ml). The so obtained yellow solution was extracted with EtOAc (3×200 mls). All the organics joined together were dried (Na₂SO₄) and evaporated the title compound D67 (2.29 g) of as yellow oil, that solidified on standing. C₁₂H₁₅BN₂O₂ requires 230. ¹H NMR (400 MHz, CDCl₃) δ ppm 7.97-8.15 (m, 1H), 7.31-7.36 (m, 1H), 3.85 (m, 4H), 2.52-2.73 (s, 3H), 0.97-1.10 (m, 6H).

### Description 68

### 6-Methyl-3-(2-pyrimidinyl)-2-pyridinecarbonitrile (D68)

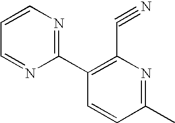

A) Isopropylmagnesium chloride-LiCl (37.9 ml, 36.5 mmol) was added portion wise (in overall 10 min) to a solution of 3-bromo-6-methyl-2-pyridinecarbonitrile (4 g, 20.30 mmol) in THF (150 ml) cooled to −70° C. (internal temperature). The reaction was kept to that temperature for 15 min. Then it was allowed to gently warm up to −40° C. in overall 1 hour. Then, it was cooled to −78° C. and zinc chloride (3.32 g, 24.36 mmol) was added. The resulting mixture was allowed to warm up to room temperature in 1 hour. Pd(Ph₃P)₄ (2.346 g, 2.030 mmol), 2-chloropyrimidine (3 g, 26.2 mmol) were added and the mixture was refluxed (external temperature 100° C.) until complete consumption of starting chloropyrimidine (3 hours). The reaction mixture was cooled to room temperature and poured into water (200 ml) cooled to 10° C. It was then extracted with EtOAc (5×200 mls). The collected organic phases, containing large amount of colloid material and water, were washed with brine (200 ml). The water phase was filtered over a gouch, and the solid material was washed with further EtOAc (2×300 mls). The collected organic phases were dried overnight over Na₂SO₄, filtered and concentrated to give (7 g) the crude material which was purified (Biotage Sp1 over a 240 g Silica Anolgix column, with a 25 g pre-column) to give the title compound D68 as yellow solid (1.8 g). UPLC (Acid GEN_QC SS): rt=0.58 minutes, peak observed: 197 (M+1). C₁₁H₈N₄ requires 196.

B) An alternative method to make D68 is: 3-(5,5-dimethyl-1,3,2-dioxaborinan-2-yl)-6-methyl-2-pyridinecarbonitrile D67 (50.6 mg) was dissolved 1,4-Dioxane (1 ml) under nitrogen in a vial, then 2-bromopyrimidine (42.0 mg, 0.264 mmol), CsF (67 mg, 0.441 mmol), Pd(Ph₃P)₄ (12 mg, 10.38 μmol) and CuI (7 mg, 0.037 mmol) were added in sequence. The vial was then capped and stirred at 65° C., after 1 hour the solvent was removed at reduced pressure and the residue partitioned between AcOEt (10 ml) and NaHCO₃ (saturated solution, 10 ml). The phases were separated and the water was extracted with AcOEt (2×10 mls). The organic fraction were joined together, dried over Na₂SO₄ and evaporated at reduced pressure, obtaining an orange oily residue which was purified (Biotage, Snap 25 g silica gel column, AcOEt/Cy from pure Cy to 50:50 in 10 column volumes) to obtain the title compound D68 as pale yellow solid (27.6 mg).

### Description 69

### 6-Methyl-3-(2-pyrimidinyl)-2-pyridinecarboxylic acid (D69)

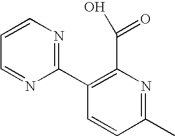

A) 6-methyl-3-(2-pyrimidinyl)-2-pyridinecarbonitrile D68 (0.8 g) was reacted in 6 M aqueous HCl (40 ml, 240 mmol) at 80° C. for 3 hours, then solvent was removed under vacuum, and the resulting crude was purified (70 g Varian C18 column conditioning with MeOH (120 mls), then water (120 mls), loading in water, washing with water (200 mls), product eluted with 100% MeOH) to give the title compound D69 (0.6 g) as yellow solid. UPLC (Acid GEN_QC SS): rt=0.30 minutes, peak observed: 216 (M+1). C₁₁H₉N₃O₂ requires 217. ¹H NMR (400 MHz, DMSO-d₆) δ ppm 13.07 (bs, 1H), 8.78-9.01 (m, 2H), 8.39 (m, 1H), 7.39-7.67 (m, 2H), 2.56-2.67 (s, 3H).

B) An alternative method to make D69 is as follows: 6-methyl-3-(2-pyrimidinyl)-2-pyridinecarbonitrile D68 (0.481 g) was suspended in EtOH (5 ml) and a solution of NaOH (0.490 g, 12.26 mmol) in water (5 ml) was added. The yellow mixture was stirred at 100° C. overnight. The yellow solution was cooled to 25° C. and HCl 6 M (1.0 ml) was added dropwise till pH=4.5. The solvent was removed to give a yellow powder that was dried at 50° C./vacuum for 1.5 hours to give the title compound D69 (1.242 g).

### Description D70

### methyl 6-methyl-3-(3-methyl-1H-pyrazol-1-yl)-2-pyridinecarboxylate (D70)

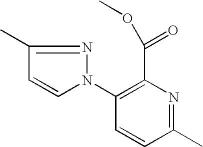

1,4-Dioxane (2 ml) was added to a mixture of methyl 3-iodo-6-methyl-2-pyridinecarboxylate D44 (50 mg), 3-methylpyrazole (17.78 mg, 0.217 mmol), (1R,2R)—N,N-dimethyl-1,2-cyclohexanediamine (5.13 mg, 0.036 mmol), copper(I) iodide (1.718 mg, 9.02 μmol) and potassium carbonate (52.4 mg, 0.379 mmol) in a screw-topped vial. The mixture was degassed via 3 vacuum/nitrogen cycles then heated to 120° C. with shaking overnight. Further (1R,2R)—N,N-dimethyl-1,2-cyclohexanediamine (5.13 mg, 0.036 mmol) and copper(I) iodide (1.718 mg, 9.02 μmol) were added and the mixture was heated to 120° C. with shaking for a further 8 hours. The reaction was filtered through a plug of silica gel washing with EtOAc. The organic phase was evaporated under reduced pressure and the residue was loaded onto a pre-conditioned SCX cartridge 1 g and the cartridge was eluted. The basic factions were evaporated under reduced pressure to give a residue which was purified by flash chromatography on silica gel (Biotage Snap 10 g column, EtOAc/Cy from 10/90 to 50/50) to give 30 mg of a 1:1.7 mixture of the title compound and 3-methylpyrazole. This material was combined with 7 mg of another batch of impure title compound prepared in a similar fashion and purified by flash chromatography on modified silica gel (Biotage KP-NH 2×Snap 11 g columns in series, EtOAc/Cy from 30/70 to 40/60) to give the title compound D70 (22 mg) as a colourless gum. UPLC (Basic QC_POS_{—}50-800): rt=0.59 minutes, peak observed: 232 (M+1). C₁₂H₁₃N₃O₂ requires 231. ¹H NMR (400 MHz, CDCl₃) δ ppm 2.38 (s, 3H) 2.68 (s, 3H) 3.89 (s, 3H) 6.28 (d, 1H) 7.38 (d, 1H) 7.63 (d, 1H) 7.80 (d, 1H).

### Description D71

### 6-methyl-3-(3-methyl-1H-pyrazol-1-yl)-2-pyridinecarboxylic acid (D71)

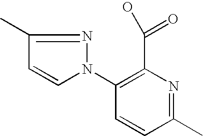

A solution of methyl 6-methyl-3-(3-methyl-1H-pyrazol-1-yl)-2-pyridinecarboxylate D70 (22 mg) and lithium hydroxide (3.42 mg, 0.143 mmol) in THF/water (2:1, 3 ml) was stirred overnight. The mixture was evaporated under reduced pressure; the residue was taken up in water (2 ml) and neutralised with 1 M HCl solution and then loaded onto a pre-conditioned C18 column (2 g, eluted with water and then MeOH). The methanol fractions were evaporated under reduced pressure to give the title compound D71 (19 mg) as a colourless gum. UPLC (Basic QC_POS_{—}50-800): rt=0.34 minutes, peak observed: 174 [(M−CO₂)+1]. C₁₁H₁₁N₃O₂ requires 217. ¹H NMR (400 MHz, methanol-d₄) δ ppm 2.34 (s, 1H) 2.66 (s, 1H) 6.35 (d, 1H) 7.56 (d, 1H) 7.85 (d, 1H) 7.94 (d, 1H).

### Description D72

### methyl 6-methyl-3-(1H-pyrazol-1-yl)-2-pyridinecarboxylate (D72)

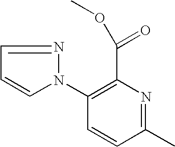

DMF (1.5 ml) was added to a mixture of methyl 3-iodo-6-methyl-2-pyridinecarboxylate D44 (200 mg), 1H-pyrazole (98 mg, 1.444 mmol), (1R,2R)—N,N-dimethyl-1,2-cyclohexanediamine (20.54 mg, 0.144 mmol), bis(copper(I) trifluoromethanesulfonate), benzene complex (18.17 mg, 0.036 mmol) and cesium carbonate (470 mg, 1.444 mmol) in a screw-topped vial. The mixture was degassed via 3 vacuum/nitrogen cycles and heated with shaking to 120° C. for 1 hour. The reaction mixture was evaporated to dryness under reduced pressure. The residue was dissolved in water/MeOH (1:1, 3 ml) and acidified to pH=2 by addition of 4 M HCl solution. The resulting mixture was evaporated to dryness under reduced pressure then the residue was triturated with DCM/MeOH (3:1, 20 ml). The mixture was filtered washing with more DCM/MeOH (3:1, 5 ml). The filtrate was treated with TMS-diazomethane solution (2 M in hexanes, 2 ml, 4 mmol) to re-esterify the acid. The reaction mixture was evaporated under reduced pressure and the residue was purified twice by flash chromatography on silica gel (Biotage Snap 10 g column, EtOAc/Cy from 20/80 to 50/50 and then Biotage KP-NH Snap 11 g column, EtOAc/DCM isocratic 1/99) to give the title compound D72 (107 mg) as a colourless gum.

UPLC (Basic QC_POS_{—}50-800): rt=0.51 minutes, peak observed: 218 (M+1). C₁₁H₁₁N₃O₂ requires 217. ¹H NMR (400 MHz, CDCl₃) δ ppm 7.63-7.86 (m, 3H), 7.39 (m, 1H), 6.48 (m, 1H), 3.85 (s, 3H), 2.68 (s, 3H).

### Description D73

### 6-methyl-3-(1H-pyrazol-1-yl)-2-pyridinecarboxylic acid (D73)

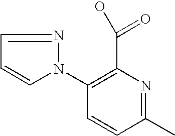

A solution of methyl 6-methyl-3-(1H-pyrazol-1-yl)-2-pyridinecarboxylate D72 (106 mg) and LiOH (17.53 mg, 0.732 mmol) in THF/water (2:1, 6 ml) was stirred overnight. The mixture was evaporated under reduced pressure; the residue was taken up in water (2 ml) and the pH was adjusted to pH=2 with 1 M HCl solution. The mixture was loaded onto a pre-conditioned C18 column (5 g, eluted with water and then MeOH). The methanol fractions were evaporated under reduced pressure to give the title compound D73 (98 mg) as a white solid.

UPLC (Basic QC_POS_{—}50-800): rt=0.30 minutes, peak observed: 160 [(M−CO₂)+1]. C₁₀H₉N₃O₂ requires 203. ¹H NMR (400 MHz, methanol-d₄) δ ppm 7.77-8.03 (m, 2H), 7.74 (m, 1H), 7.58 (m, 1H), 6.55 (m, 1H), 2.66 (s, 3H).

### Description D74

### methyl 3-(4,5-dimethyl-2H-1,2,3-triazol-2-yl)-6-methyl-2-pyridinecarboxylate (D74)

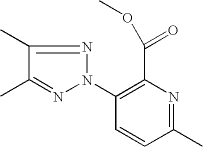

DMF (1.5 ml) was added to a mixture of methyl 3-iodo-6-methyl-2-pyridinecarboxylate D44 (50 mg), 4,5-dimethyl-1H-1,2,3-triazole (Chem Ber, 1966, p 2512) (21.91 mg, 0.226 mmol), (1R,2R)—N,N′-dimethyl-1,2-cyclohexanediamine (5.13 mg, 0.036 mmol), bis(copper(I) trifluoromethanesulfonate), benzene complex (4.54 mg, 9.02 μmol) and cesium carbonate (118 mg, 0.361 mmol) in a screw-topped vial. The mixture was degassed via 3 vacuum/nitrogen cycles and heated with shaking to 120° C. for 9 hours. The reaction mixture was evaporated to dryness under reduced pressure. The residue was dissolved in water/MeOH (1:1, 3 ml) and acidified to pH=2 by addition of 4 M HCl solution. The resulting mixture was evaporated to dryness under reduced pressure then the residue was triturated with DCM/MeOH (3:1, 5 ml). The mixture was filtered washing with more DCM/MeOH (3:1, 5 ml). The filtrate was treated with trimethylsilyldiazomethane solution (2 M in hexanes, 2 ml, 4 mmol) to re-esterify the acid. The reaction mixture was evaporated under reduced pressure and the residue was purified by flash chromatography on silica gel (Biotage Snap 10 g column, EtOAc/Cy from 20/80 to 50/50) to give the title compound D74 (22 mg) as a colourless solid. UPLC (Acid QC_POS_{—}50-800): rt=0.63 minutes, peak observed: 247 (M+1). C₁₂H₁₄N₄O₂ requires 246. ¹H NMR (400 MHz, CDCl₃) δ ppm 2.33 (s, 6H) 2.66 (s, 3H) 3.95 (s, 3H) 7.36 (d, 1H) 8.14 (d, 1H).

### Description D75

### 3-(4,5-dimethyl-2H-1,2,3-triazol-2-yl)-6-methyl-2-pyridinecarboxylic acid (D75)

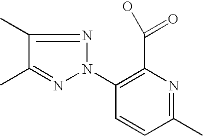

A solution of methyl 3-(4,5-dimethyl-2H-1,2,3-triazol-2-yl)-6-methyl-2-pyridinecarboxylate D74 (22 mg) and lithium hydroxide (3.21 mg, 0.134 mmol) in THF/water (2:1, 3 ml) was stirred overnight. The mixture was evaporated under reduced pressure; the residue was taken up in water (2 ml) and the pH was adjusted to pH=2 with 1M HCl solution. The mixture was loaded onto a pre-conditioned C18 column (5 g, eluted with water and then MeOH). The methanol fractions were evaporated under reduced pressure to give the title compound D75 (20 mg) as a white solid. UPLC (Acid QC_POS_{—}50-800): rt=0.46 minutes, peaks observed: 233 (M+1) and 189 [(M−CO₂)+1]. C₁₁H₁₂N₄O₂ requires 232. ¹H NMR (400 MHz, CDCl₃) δ ppm 2.37 (s, 6H) 2.72 (s, 3H) 7.51 (d, 1H) 7.99 (d, 1H).

### Description D76

### 4-(bromomethyl)-1-(phenylmethyl)-1H-1,2,3-triazole (D76)

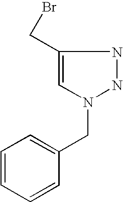

Triphenylphosphine (2.204 g, 8.40 mmol) and carbon tetrabromide (2.79 g, 8.40 mmol) were added to a stirred solution of [1-(phenylmethyl)-1H-1,2,3-triazol-4-yl]methanol (Synthetic Commun. 2007, 37, 805-812) (1.06 g, 5.60 mmol) in DCM (50 ml) at room temperature and the resulting mixture was stirred overnight (˜18 hours). The reaction mixture was evaporated under reduced pressure and the residue was purified by flash chromatography on silica gel (Biotage Snap 100 g column, EtOAc/DCM from 2/98 to 5/95) to give the title compound D76 (1.27 g) as a white solid.

UPLC (Acid QC_POS_{—}50-800): rt=0.63 minutes, peaks observed: 252 and 254 (M+1). C₁₀H₁₀BrN₃ requires 251 and 253. ¹H NMR (400 MHz, CDCl₃) δ ppm 4.58 (s, 2H) 5.55 (s, 2H) 7.30-7.34 (m, 2H) 7.38-7.46 (m, 3H) 7.51 (s, 1H).

### Description D77

### 4-methyl-1H-1,2,3-triazole (D77)

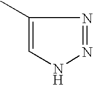

A slurry of 10% palladium on carbon (wet) (355 mg, 0.167 mmol) in EtOH (2 ml) was added to a stirred solution of 4-(bromomethyl)-1-(phenylmethyl)-1H-1,2,3-triazole D76 (700 mg) under nitrogen and the resulting mixture was stirred under an atmosphere of hydrogen gas overnight (˜20 hours). The reaction mixture was filtered through a plug of celite washing with MeOH. The filtrate was evaporated under reduced pressure to give ˜500 mg of a yellow solid residue which was purified by SCX cartridge (10 g) to give the title compound D77 (223 mg) as a colourless liquid. UPLC (Acid QC_POS_{—}50-800): rt=0.29 minutes, peaks observed: 84 (M+1). C₃H₅N₃ requires 83. ¹H NMR (400 MHz, CDCl₃) δ ppm 2.42 (s, 3H) 7.53 (s, 1H).

### Description D78

### methyl 6-methyl-3-(4-methyl-2H-1,2,3-triazol-2-yl)-2-pyridinecarboxylate (D78)

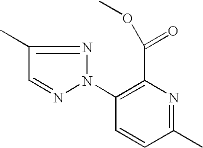

DMF (1.5 ml) was added to a mixture of methyl 3-iodo-6-methyl-2-pyridinecarboxylate D44 (200 mg), 4-methyl-1H-1,2,3-triazole D77 (120 mg), (1R,2R)—N,N′-dimethyl-1,2-cyclohexanediamine (20.54 mg, 0.144 mmol), copper(I) trifluoromethanesulfonate benzene complex (18.17 mg, 0.036 mmol) and cesium carbonate (470 mg, 1.444 mmol) in a screw-topped vial. The mixture was degassed via 3 vacuum/nitrogen cycles and heated with shaking to 120° C. for 5 hours. The reaction mixture was evaporated to dryness under reduced pressure. The residue was dissolved in water/MeOH (1:1, 3 ml) and acidified to pH=2 by addition of 2 M HCl solution. The resulting mixture was evaporated to dryness under reduced pressure then the residue was triturated with DCM/MeOH (3:1, 5 ml). The mixture was filtered washing with more DCM/MeOH (3:1, 5 ml). The filtrate was treated with TMS-diazomethane solution (2 M in hexanes, 4 ml, 8 mmol) to re-esterify the acid. The reaction mixture was evaporated under reduced pressure and the residue was purified by flash chromatography on silica gel (Biotage Snap 25 g column, EtOAc/Cy from 20/80 to 50/50) to give the title compound D78 (121 mg) as a colourless solid. UPLC (Acid QC_POS_{—}50-800): rt=0.59 minutes, peak observed: 233 (M+1). C₁₁H₁₂N₄O₂ requires 232. ¹H NMR (400 MHz, CDCl₃) δ ppm 8.15 (d, 1H), 7.59 (s, 1H), 7.37 (d, 1H), 3.92 (s, 3H), 2.66 (s, 3H), 2.40 (s, 3H).

### Description D79

### 6-methyl-3-(4-methyl-2H-1,2,3-triazol-2-yl)-2-pyridinecarboxylic acid (D79)

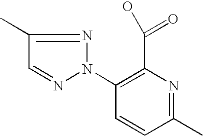

A solution of methyl 6-methyl-3-(4-methyl-2H-1,2,3-triazol-2-yl)-2-pyridinecarboxylate D78 (120 mg) and lithium hydroxide (18.56 mg, 0.775 mmol) in THF/water (2:1, 4.5 ml) was stirred for 2 hours. The mixture was stirred for another 2 hours then evaporated under reduced pressure; the residue was taken up in water (3 ml) and the pH was adjusted to pH=2 with 1 M HCl solution. The mixture was loaded onto a pre-conditioned C18 column (10 g, eluted with water and then MeOH). The methanol fractions were evaporated under reduced pressure to give the title compound D79 (109 mg) as a white solid. UPLC (Acid QC_POS_{—}50-800): rt=0.59 minutes, peak observed: 219 (M+1). C₁₀H₁₀N₄O₂ requires 218. ¹H NMR (400 MHz, CDCl₃) δ ppm 8.00 (d, 1H), 7.65 (s, 1H), 7.52 (d, 1H), 2.71 (s, 3H), 2.43 (s, 3H).

### Description D80

### 6-methyl-3-(2-methyl-4-pyrimidinyl)-2-pyridinecarbonitrile (D80)

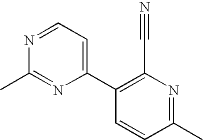

Pd(Ph₃P)₄ (37.7 mg, 0.033 mmol) was added to a mixture of 4-chloro-2-methylpyrimidine (117 mg, 0.913 mmol), 3-(5,5-dimethyl-1,3,2-dioxaborinan-2-yl)-6-methyl-2-pyridinecarbonitrile D67 (150 mg), copper(I) iodide (22.35 mg, 0.117 mmol) and cesium fluoride (198 mg, 1.304 mmol) in 1,4-dioxane (3 ml) at room temperature. The mixture was degassed via 3 vacuum/nitrogen cycles and sonicated briefly to homogenise the reaction mixture which was then heated to 65° C. with shaking for 1 hour. The mixture was cooled and filtered washing with EtOAc. The organic phase was evaporated under reduced pressure. The residue was taken up in EtOAc (30 ml) and washed with NaHCO₃ solution, dried (Na₂SO₄) and evaporated under reduced pressure. This residue was purified twice by flash chromatography on silica gel (Biotage Snap 25 g column, EtOAc/Cy from 50/50 to 100/0 then Biotage Snap 25 g column, isocratic Et₂O) to give 85 mg of almost pure title compound. This material was purified further by recrystallisation from EtOH/Cy to give the title compound D80 (59 mg) as a yellow solid.

UPLC (Acid QC_POS_{—}50-800): rt=0.53 minutes, peak observed: 211 (M+1). C₁₂H₁₀N₄ requires 210. ¹H NMR (400 MHz, CDCl₃) δ ppm 2.72 (s, 3H) 2.87 (s, 3H) 7.55 (d, 1H) 7.75 (d, 1H) 8.24 (d, 1H) 8.85 (d, 1H).

### Description D81

### 6-methyl-3-(2-methyl-4-pyrimidinyl)-2-pyridinecarboxylic acid (D81)

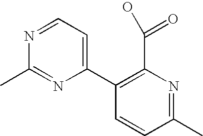

NaOH (39.3 mg, 0.982 mmol) in water (1 ml) was added to a suspension of 6-methyl-3-(2-methyl-4-pyrimidinyl)-2-pyridinecarbonitrile D80 (59 mg) in EtOH (1.5 ml) and the resulting mixture was heated to 100° C. with shaking for 1 hour, then at 60° C. overnight. Further NaOH (10 mg, 0.25 mmol) was added and the reaction was shaken at 100° C. for 4 hours. The mixture was evaporated under reduced pressure, then the residue was taken up in water (1.5 ml) and EtOH (0.5 ml), further NaOH (10 mg, 0.25 mmol) was added and the mixture was heated to 100° C. with shaking for 3 hours. The mixture was evaporated under reduced pressure; the residue was taken up in water (2 ml) and acidified to pH=2 with 2 M HCl solution. This mixture was loaded onto a pre-conditioned C18 cartridge (5 g, eluted with water and then MeOH). The MeOH fractions were evaporated under reduced pressure to give the title compound D81 (64 mg) as an off white solid. UPLC (Acid QC_POS_{—}50-800): rt=0.33 minutes, peak observed: 186 [(M−CO₂)+1]. C₁₂H₁₁N₃O₂ requires 229. ¹H NMR (400 MHz, CDCl₃) δ ppm 2.73 (s, 3H) 2.82 (s, 3H) 7.34 (d, 1H) 7.56 (d, 1H) 7.89 (d, 1H) 8.72 (d, 1H).

### Description D82

### 6,6′-dimethyl-2,3′-bipyridine-2′-carbonitrile (D82)

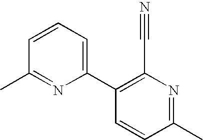

Pd(Ph₃P)₄ (37.7 mg, 0.033 mmol) was added to a mixture of 2-bromo-6-methylpyridine (157 mg, 0.913 mmol), 3-(5,5-dimethyl-1,3,2-dioxaborinan-2-yl)-6-methyl-2-pyridinecarbonitrile D67 (150 mg), copper(I) iodide (22.35 mg, 0.117 mmol) and cesium fluoride (198 mg, 1.304 mmol) in 1,4-dioxane (3 ml) at room temperature. The mixture was degassed via 3 vacuum/nitrogen cycles and sonicated briefly to homogenise the reaction mixture which was then heated to 65° C. with shaking for 2 hours. The mixture was cooled and filtered washing with EtOAc. The organic phase was evaporated under reduced pressure. This residue was purified by flash chromatography on silica gel (Biotage Snap 25 g column, EtOAc/Cy from 30/70 to 50/50) to give the title compound D82 (62 mg) as a pale yellow solid.

UPLC (Acid QC_POS_{—}50-800): rt=0.60 minutes, peak observed: 210 (M+1). C₁₃H₁₁N₃ requires 209. ¹H NMR (400 MHz, CDCl₃) δ ppm 2.69 (s, 3H) 2.69 (s, 3H) 7.27 (d, 1H) 7.49 (d, 1H) 7.69 (d, 1H) 7.77 (t, 1H) 8.14 (d, 1H).

### Description D83

### 6-methyl-3-(2-methyl-4-pyrimidinyl)-2-pyridinecarboxylic acid (D83)

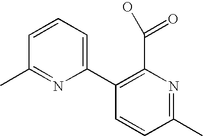

NaOH (46.6 mg, 1.166 mmol) in water (1 ml) was added to a suspension of 6,6′-dimethyl-2,3′-bipyridine-2′-carbonitrile D82 (61 mg) in EtOH (1.5 ml) and the resulting mixture was heated to 100° C. with shaking for 6 hours. The mixture was evaporated under reduced pressure, the residue was taken up in water (2 ml) and acidified to pH=2 with 2M HCl solution. This mixture was loaded onto a pre-conditioned C18 cartridge (10 g, eluted with water and then MeOH). The MeOH fractions were evaporated under reduced pressure to give the title compound D83 (66 mg) as a pale yellow solid.

UPLC (Acid QC_POS_{—}50-800): rt=0.33 minutes, peak observed: 185 [(M−CO₂)+1]. C₁₃H₁₂N₂O₂ requires 228. ¹H NMR (400 MHz, CDCl₃) δ ppm 2.65 (s, 3H) 2.71 (s, 3H) 7.24 (d, 1H) 7.34 (d, 1H) 7.50 (d, 1H) 7.71 (t, 1H) 7.89 (d, 1H).

### Description D84

### methyl 6-methyl-3-(3-methyl-1H-1,2,4-triazol-1-yl)-2-pyridinecarboxylate (D84)

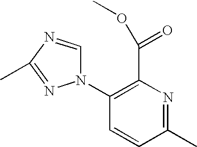

DMF (1.5 ml) was added to a mixture of methyl 3-iodo-6-methyl-2-pyridinecarboxylate D44 (100 mg), 3-methyl-1H-1,2,4-triazole (45.0 mg, 0.541 mmol), (1R,2R)—N,N′-dimethyl-1,2-cyclohexanediamine (10.27 mg, 0.072 mmol), copper(I) trifluoromethanesulfonate benzene complex (9.08 mg, 0.018 mmol) and cesium carbonate (235 mg, 0.722 mmol) in a screw-topped vial. The mixture was degassed via 3 vacuum/nitrogen cycles and heated with shaking to 120° C. for 90 minutes. The reaction mixture was evaporated to dryness under reduced pressure. The residue was dissolved in water/MeOH (1:1, 3 ml) and acidified to pH=2 by addition of 2 M HCl solution. The resulting mixture was evaporated to dryness under reduced pressure then the residue was triturated with DCM/MeOH (3:1, 5 ml). The mixture was filtered washing with more DCM/MeOH (3:1, 5 ml). The filtrate was treated with trimethylsilyldiazomethane solution (2 M in hexanes, 2 ml, 4 mmol) to re-esterify the acid. The reaction mixture was evaporated under reduced pressure and the residue was purified by flash chromatography on silica gel (Biotage Snap 2×10 g columns in series, EtOAc/Cy from 50/50 to 100/0) to give the title compound D84 (48 mg) as a colourless solid. UPLC (Acid FINAL_QC_POS): rt=0.45 minutes, peak observed: 233 (M+1). C₁₁H₁₂N₄O₂ requires 232. ¹H NMR (400 MHz, CDCl₃) δ ppm 2.52 (s, 3H) 2.73 (s, 3H) 3.91 (s, 3H) 7.47 (d, 1H) 7.81 (d, 1H) 8.32 (s, 1H).

### Description D85

### 6-methyl-3-(3-methyl-1H-1,2,4-triazol-1-yl)-2-pyridinecarboxylic acid (D85)

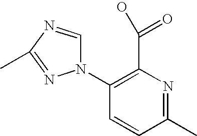

A solution of methyl 6-methyl-3-(3-methyl-1H-1,2,4-triazol-1-yl)-2-pyridinecarboxylate D84 (48 mg) and lithium hydroxide (7.42 mg, 0.310 mmol) in THF/water (2:1, 4.5 ml) was stirred for 1 hour. The mixture was evaporated under reduced pressure and the residue was taken up in water (2 ml) and the pH was adjusted to pH=2 with 1 M HCl solution. The mixture was loaded onto a pre-conditioned C18 column (5 g, eluted with water and then MeOH). The methanol fractions were evaporated under reduced pressure to give the title compound D85 (45 mg) as a white solid.

UPLC (Acid GEN_C): rt=0.34 minutes, peaks observed: 219 (M+1) and 175 [(M−CO₂)+1]. C₁₀H₁₀N₄O₂ requires 218. ¹H NMR (400 MHz, CDCl₃) δ ppm 2.53 (s, 3H) 2.74 (s, 3H) 7.61 (d, 1H) 8.05 (d, 1H) 8.60 (s, 1H).

### Description D86

### 3-(5-fluoro-2-pyrimidinyl)-6-methyl-2-pyridinecarbonitrile (D86)

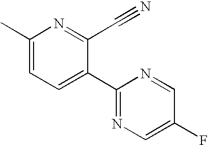

3-(5,5-dimethyl-1,3,2-dioxaborinan-2-yl)-6-methyl-2-pyridinecarbonitrile D67 (130 mg) 2-bromo-5-fluoropyrimidine (150 mg, 0.678 mmol), cesium fluoride (172 mg, 1.130 mmol), copper(I) iodide (18.19 mg, 0.095 mmol), Pd(Ph₃P)₄ (32.6 mg, 0.028 mmol) were suspended in 1,4-Dioxane (2.25 ml) and stirred at 65° C. for 1.5 hours. After this time the reaction mixture was filtered through a celite pad, rinsed with EtOAc (20 ml) and the organic solution was evaporated under reduced pressure to give a dark orange semisolid which was purified by silica gel chromatography (SNAP KP-Sil 25 g cartridge; eluted with Cy/EtOAc: from 100/0 to 70/30). Collected and evaporated fractions gave the title compound D86 like a yellowish solid (65 mg). UPLC (Basic GEN_C): rt=0.64 minutes, peak observed 215(M+1), C₁₁H₇FN₄ requires 214. ¹H NMR (400 MHz, DMSO-d₆) δ ppm 2.62 (s, 3H) 7.76 (d, 1H) 8.57 (d, 1H) 9.14 (s, 2H).

### Description D87

### 3-(5-fluoro-2-pyrimidinyl)-6-methyl-2-pyridinecarboxylic acid (D87)

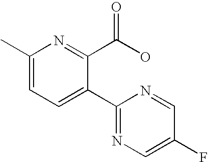

3-(5-fluoro-2-pyrimidinyl)-6-methyl-2-pyridinecarbonitrile D86 (63 mg) was dissolved in HCl 6 M in water (3 ml, 18.00 mmol) and stirred at 100° C. for 3.5 hours. The solvent was removed under reduced pressure and the brown solid obtained was charged on a inverse phase cartridge (C18, 20 g), washed with water (225 ml) and eluted with MeOH (50 ml). The organic fraction was evaporated under vacuum giving a yellow oil (55 mg) which was triturated with Et₂O (1 ml) and a yellow solid resulted title compound D87 (43 mg). UPLC (Acid IPQC): rt=0.36 minutes, peak observed 232 (M−1), C₁₁H₈FN₃O₂ requires 233. ¹H NMR (400 MHz, DMSO-d₆) δ ppm 2.56 (s, 3H) 7.51 (d, 1H) 8.32 (d, 1H) 9.01 (s, 2H) 13.16 (br. s., 1H)

### Description D88

### 6-methyl-3-[5-(trifluoromethyl)-2-pyrimidinyl]-2-pyridinecarbonitrile (D88)

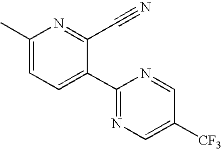

3-(5,5-dimethyl-1,3,2-dioxaborinan-2-yl)-6-methyl-2-pyridinecarbonitrile D67 (130 mg) 2-chloro-5-(trifluoromethyl)pyrimidine (124 mg, 0.678 mmol), cesium fluoride (172 mg, 1.130 mmol), copper(I) iodide (18.19 mg, 0.095 mmol), Pd(Ph₃P)₄ (32.6 mg, 0.028 mmol) were suspended in 1,4-Dioxane (2.25 mL) and stirred at 65° C. for 1 hours. After this time the reaction mixture was filtered through a celite pad, rinsed with EtOAc (20 ml) and the organic solution was evaporated under reduced pressure to give a dark orange oil which was purified by silica gel chromatography (SNAP KP-Sil 25 g cartridge; eluted with Cy/EtOAc: 100% Cy, to 80/20 Cy/EtOAc) to give the title compound D88 like a yellow solid (63 mg). UPLC (Basic GEN_QC): rt=0.79 minutes, peak observed 265 (M+1), C₁₂H₇F₃N₄ requires 264. ¹H NMR (400 MHz, DMSO-d₆) δ ppm 2.64 (s, 3H) 7.82 (d, 1H) 8.67 (d, 1H) 9.52 (s, 2H).

### Description D89

### 6-methyl-3-[5-(trifluoromethyl)-2-pyrimidinyl]-2-pyridinecarboxylic acid (D89)

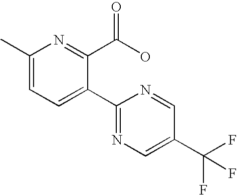

6-methyl-3-[5-(trifluoromethyl)-2-pyrimidinyl]-2-pyridinecarbonitrile D88 (63 mg) was dissolved in HCl 6 M in water (3 ml, 18.00 mmol) and stirred at 100° C. for 1.5 hours. The solvent was removed under reduced pressure and the brown solid obtained was charged on a inverse phase cartridge C18 (20 g, washed with water and eluted with MeOH) to give title compound D89 like yellow solid (32 mg). UPLC (Acid IPQC): rt=0.57 minutes, peak observed 282 (M−1), C₁₂H₈F₃N₃O₂ requires 283. ¹H NMR (400 MHz, DMSO-d₆) δ ppm 2.59 (s, 3H) 7.57 (d, 1H) 8.40 (d, 1H) 9.37 (s, 2H) 13.20 (br. s., 1H)

### Description D90

### 6-methyl-3-(3-pyridazinyl)-2-pyridinecarbonitrile (D90)

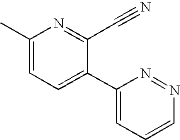

3-(5,5-dimethyl-1,3,2-dioxaborinan-2-yl)-6-methyl-2-pyridinecarbonitrile D67 (150 mg) 3-chloropyridazine (74.7 mg, 0.652 mmol), cesium fluoride (198 mg, 1.304 mmol), copper(I) iodide (20.98 mg, 0.110 mmol), Pd(Ph₃P)₄ (37.7 mg, 0.033 mmol) were suspended in 1,4-Dioxane (2.6 ml) and stirred at 65° C. for 1.5 hours. After this time the reaction mixture was filtered through a celite pad, rinsed with EtOAc (20 ml) and the organic solution was evaporated under reduced pressure to give a black oil which was purified by silica gel chromatography (SNAP KP-Sil 25 g cartridge; eluted with Cy/EtOAc: 100% Cy to 80/20 Cy/EtOAc). Collected and evaporated fractions gave title compound D90 (61 mg) as yellow solid. UPLC (Basic GEN_QC): rt=0.47 minutes, peak observed 197 (M+1), C₁₁H₈N₄ requires 196. ¹H NMR (400 MHz, DMSO-d₆) δ ppm 2.64 (s, 3H) 7.80 (d, 1H) 7.94 (dd, 1H) 8.22 (dd, 1H) 8.32 (d, 1H) 9.38 (dd, 1H).

### Description D91

### 6-methyl-3-(3-pyridazinyl)-2-pyridinecarboxylic acid (D91)

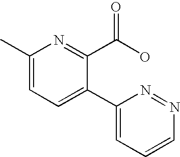

In a 20 ml screw cap vial, NaOH (87 mg, 2.176 mmol) was added to a suspension of 6-methyl-3-(3-pyridazinyl)-2-pyridinecarbonitrile D90 (61 mg) in EtOH (7 ml) and water (6 ml) and the mixture was stirred at 100° C. for 1.5 hours. The solvent was removed at reduced pressure. The residue was dissolved in water (4 ml) and this solution was washed with Et₂O (3×3 ml). After the separation, the pH of the aqueous layer was adjusted to about pH=4 with HCl 6 M. This solution was charged on a inverse phase cartridge C18 (25 g, washed with water and then with MeOH). The desired compound was not kept by the cartridge and it was recovered, together with salts, in the water fractions, which were evaporated under reduced pressure. The yellow solid obtained was dissolved in water (4 ml) and aqueous 1 M HCl (1.2 ml), giving a solution with a pH between 1 and 2. That solution was charged on a inverse phase cartridge C18 (25 g, washed with water then eluted with water and successively with water/MeOH 80/20). A white solid was obtained and resulted to be title compound D91, (42 mg). UPLC (Acid IPQC): rt=0.28 minutes, peak observed 214 (M−1), C₁₁H₉N₃O₂ requires 215. ¹H NMR (400 MHz, DMSO-d₆) δ ppm 2.58 (s, 3H) 7.55 (d, 1H) 7.76-7.84 (m, 1H) 7.94-8.02 (m, 1H) 8.07 (d, 1H) 9.18-9.30 (m, 1H) 13.06 (br. s., 1H).

### Description D92

### 6′-methyl-2,3′-bipyridine-2′-carbonitrile (D92)

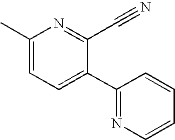

3-(5,5-dimethyl-1,3,2-dioxaborinan-2-yl)-6-methyl-2-pyridinecarbonitrile D67 (150 mg) 2-bromopyridine (0.062 mL, 0.652 mmol), cesium fluoride (198 mg, 1.304 mmol), copper(I) iodide (20.98 mg, 0.110 mmol), Pd(Ph₃P)₄ (37.7 mg, 0.033 mmol) were suspended in 1,4-Dioxane (2.25 ml) and stirred at 65° C. for 1 hours. After this time the reaction mixture was filtered through a celite pad, rinsed with EtOAc (20 ml) and the organic solution was evaporated under reduced pressure to give a dark orange oil which was purified by silica gel chromatography (SNAP KP-Sil 25 g cartridge; eluted with Cy/EtOAc: 100% Cy to 80/20 Cy/EtOAc). Collected and evaporated fractions gave a yellow solid, (65 mg) resulted to be title compound D92. UPLC (Basic GEN_QC): rt=0.59 minutes, peak observed 196 (M+1), C₁₂H₉N₃ requires 195. ¹H NMR (400 MHz, DMSO-d₆) δ ppm 2.60 (s, 3H) 7.51-7.58 (m, 1H) 7.73 (d, 1H) 7.86-7.98 (m, 1H) 7.99-8.09 (m, 1H) 8.25 (d, 1H) 8.77 (d, 1H).

### Description D93

### 6′-methyl-2,3′-bipyridine-2′-carboxylic acid (D93)

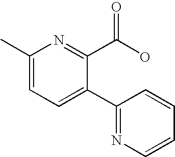

6′-methyl-2,3′-bipyridine-2′-carbonitrile D92 (65 mg) was suspended in EtOH (0.7 ml) into a 20 ml-vial, then a solution of NaOH (93 mg, 2.331 mmol) in water (0.6 ml) was added (the system became brilliant yellow), the vial was capped and the mixture was stirred at 100° C. after 5 hours the solvent was removed at reduced pressure. The residue was dissolved in water (4 ml) and this solution was washed with Et₂O. After the separation, the pH of the aqueous layer was adjusted to about 4 with HCl 6 M. This solution was charged on a inverse phase cartridge C18 (25 g washed with water and then with MeOH). The title compound D93 (66 mg) was recovered (in the water fractions which were evaporated under reduced pressure). UPLC (Acid IPQC): rt=0.31 minutes, peak observed 213 (M−1), C₁₂H₁₀N₂O₂ requires 214. ¹H NMR (400 MHz, DMSO-d₆) δ ppm 2.55 (s, 3H) 7.34-7.44 (m, 1H) 7.47 (d, 1H) 7.69 (d, 1H) 7.85-7.96 (m, 1H) 8.04 (d, 1H) 8.61 (d, 1H) 12.98 (br. s., 1H).

### Description D94

### 6-methyl-3-(2-pyrazinyl)-2-pyridinecarbonitrile (D94)

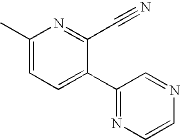

3-(5,5-dimethyl-1,3,2-dioxaborinan-2-yl)-6-methyl-2-pyridinecarbonitrile D67 (150 mg), 2-iodopyrazine (0.064 ml, 0.652 mmol), cesium fluoride (198 mg, 1.304 mmol), copper(I) iodide (20.98 mg, 0.110 mmol), Pd(Ph₃P)₄ (37.7 mg, 0.033 mmol) were suspended in 1,4-Dioxane (2.25 ml) and stirred at 65° C. for 1 hours. After this time the reaction mixture was filtered through a celite pad, rinsed with EtOAc (20 ml) and the organic solution was evaporated under reduced pressure to give a dark orange oil which was purified by silica gel chromatography (SNAP KP-Sil 25 g cartridge; eluted with Cy/EtOAc: from 100/0 to 80/20) to give the title compound D94 (100 mg) as yellow solid. UPLC (Basic GEN_QC): rt=0.53 minutes, peak observed 197 (M+1), C₁₁H₈N₄ requires 196. ¹H NMR (400 MHz, DMSO-d₆) δ ppm 2.62 (s, 3H) 7.79 (d, 1H) 8.36 (d, 1H) 8.80 (d, 2H) 9.19 (d, 1H).

### Description D95

### 6-methyl-3-(2-pyrazinyl)-2-pyridinecarboxylic acid (D95)

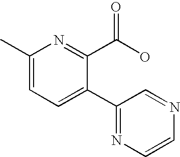

In a 20 ml screw cap vial, NaOH (143 mg) was added to a suspension of 6-methyl-3-(2-pyrazinyl)-2-pyridinecarbonitrile D94 (100 mg, 0.510 mmol) in EtOH (1.16 ml) and water (1 ml) and the mixture was stirred at 100° C. for 1.5 hours. The solvent was removed at reduced pressure. The residue was dissolved in water (4 ml) and this solution was washed with Et₂O (3×3 ml). The aqueous layer was evaporated under reduced pressure. The dark green solid obtained was dissolved in water (3 ml) and aqueous 1 M HCl (2.7 ml), giving a solution with a pH between 1 and 2. That solution was charged on a inverse phase cartridge C18 (25 g washed with water then eluted with water and successively with water/MeOH 80/20). A white solid was obtained and resulted to be the title compound D95 (26 mg). UPLC (Acid IPQC): rt=0.31 minutes, peak observed 214 (M−1), C₁₁H₉N₃O₂ requires 214. ¹H NMR (400 MHz, DMSO-d₆) δ ppm 2.57 (s, 3H) 7.53 (d, 1H) 8.12 (d, 1H) 8.65 (d, 1H) 8.67-8.72 (m, 1H) 8.92-8.99 (m, 1H) 13.21 (br. s., 1H).

### Description 96

### 6-methyl-3-(5-methyl-2-pyrimidinyl)-2-pyridinecarbonitrile (D96)

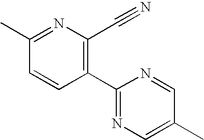

3-(5,5-dimethyl-1,3,2-dioxaborinan-2-yl)-6-methyl-2-pyridinecarbonitrile D67 (154 mg) was dissolved 1,4-dioxane (3 ml) under nitrogen into an 8 ml-vial, then 2-chloro-5-methylpyrimidine (119 mg, 0.926 mmol), cesium fluoride (204 mg, 1.343 mmol), Pd(Ph₃P)₄ (37 mg, 0.032 mmol) and copper(I) iodide (22 mg, 0.116 mmol) were added in sequence. The vial was then capped, the white solid on the bottom of the vial was crumbled by ultra-sound action for 30 seconds and then the gray slurry was stirred at 65° C. after 1 hour the solvent was evaporated at reduced pressure and the dark residue stored into the fridge overnight. The residue was then partitioned between DCM and sodium bicarbonate (saturated solution, 30 ml). The phases were separated and the watery one was extracted with DCM. All the organic fraction were joined together, dried over sodium sulphate and evaporated at reduced pressure, obtaining a brown/orange oily residue which was purified by Biotage (Snap 25 g silica gel column, EtOAc/Cy from pure Cy to 60:40 in 15 CV), it was obtained the title compound D96 (65 mg) as pale yellow solid. UPLC (Acid GEN_C): rt=0.59 minutes, peak observed: 211 (M+1). C₁₂H₁₀N₄ requires 210.

¹H NMR (400 MHz, CDCl₃) δ ppm 2.43 (s, 3H) 2.70 (s, 3H) 7.49 (d, 1H) 8.55 (d, 1H) 8.77 (s, 2H).

### Description 97

### 6-methyl-3-(5-methyl-2-pyrimidinyl)-2-pyridinecarboxylic acid (D97)

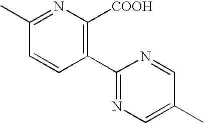

6-methyl-3-(5-methyl-2-pyrimidinyl)-2-pyridinecarbonitrile D96 (62 mg) was suspended in EtOH (0.7 ml) into a 20 ml-vial, then a solution of NaOH (83 mg, 2.075 mmol) in water (0.6 ml) was added (the system became brilliant yellow), the vial was capped and the mixture was stirred at 100° C. after 5 hours the solvent was removed at reduced pressure.

The residue was dissolved in water (4 ml) and this solution was washed with Et₂O, in order to eliminate the most of the primary amide; then the pH of the water solution was adjusted to about 3 with HCl 1 M: no precipitation occurred during the acidification.

The whole solution was loaded onto a Varian Mega-Bond C18-25 g column (after washing the column with about 1 CV of water, the product was collected eluting with ACN 25 ml) it was obtained the title compound D97 (60 mg) as white solid. UPLC (Acid GEN_C): rt=0.35 minutes, peak observed: 230 (M+1). C₁₂H₁₁N₃O₂ requires 229. ¹H NMR (400 MHz, DMSO-d₆) δ ppm 2.33 (s, 3H) 2.56 (s, 3H) 7.49 (d, 1H) 8.36 (d, 1H) 8.75 (s, 2H) 13.05 (br. s., 1H)

### Description 98

### 3-(4,6-dimethyl-2-pyrimidinyl)-6-methyl-2-pyridinecarbonitrile (D98)

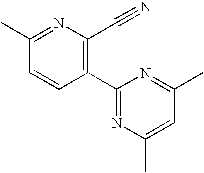

3-(5,5-dimethyl-1,3,2-dioxaborinan-2-yl)-6-methyl-2-pyridinecarbonitrile D67 (154 mg) was dissolved 1,4-Dioxane (3 ml) under nitrogen into an 8 ml-vial, then 2-chloro-4,6-dimethylpyrimidine (133 mg, 0.933 mmol), cesium fluoride (204 mg, 1.343 mmol), Pd(Ph₃P)₄ (37 mg, 0.032 mmol) and copper(I) iodide (22 mg, 0.116 mmol) were added in sequence. The vial was then capped, the white solid on the bottom of the vial was crumbled by ultra-sound action for 30 seconds and then the gray slurry was stirred at 65° C. after 1 hour the solvent was evaporated at reduced pressure and the dark residue stored into the fridge overnight. The residue was then partitioned between DCM and sodium bicarbonate (saturated solution, 30 ml). The phases were separated and the watery one was extracted with DCM. All the organic fraction were joined together, dried over sodium sulphate and evaporated at reduced pressure, obtaining a brown/orange oily residue which was purified by Biotage (Snap 25 g silica gel column, EtOAc/Cy from pure Cy to 60:40 in 15 CV) it was obtained the title compound D98 (80 mg) as pale yellow solid. UPLC (Acid GEN_C): rt=0.63 minutes, peak observed: 225 (M+1). C₁₃H₁₂N₄ requires 224. ¹H NMR (400 MHz, CDCl₃) δ ppm 2.60 (s, 6H) 2.69 (s, 3H) 7.08 (s, 1H) 7.47 (d, 1H) 8.57 (d, 1H)

### Description 99

### 3-(4,6-dimethyl-2-pyrimidinyl)-6-methyl-2-pyridinecarboxylic acid (D99)

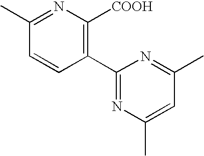

3-(4,6-dimethyl-2-pyrimidinyl)-6-methyl-2-pyridinecarbonitrile D98 (78 mg) was suspended in EtOH (0.8 mL) into a 20 ml-vial, then a solution of NaOH (98 mg, 2.450 mmol) in water (0.7 ml) was added (became brilliant yellow), the vial was capped and the mixture was stirred at 100° C., after 5 hours the solvent was removed at reduced pressure. The residue was dissolved in water (4 ml) and this solution was washed with Et₂O, in order to eliminate the most of the primary amide; then the pH of the water solution was adjusted to about pH=3 with HCl 1 M: no precipitation occurred during the acidification. The whole solution was loaded onto a Varian Mega-Bond C18-25 g column (after washing the column with about 1 CV of water, the product was collected eluting with ACN 25 ml) it was obtained the titled compound D99 (67 mg) as white solid. UPLC (Acid GEN_C): rt=0.37 minutes, peak observed: 244 (M+1). C₁₃H₁₃N₃O₂ requires 243. ¹H NMR (400 MHz, DMSO-d₆) δ ppm 2.46 (s, 6H) 2.55 (s, 3H) 7.24 (s, 1H) 7.47 (d, 1H) 8.37 (d, 1H) 12.97 (br. s., 1H)

### Description 100

### 6-methyl-3-(4-methyl-2-pyrimidinyl)-2-pyridinecarboxylic acid (D100)

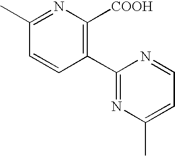

3-(5,5-dimethyl-1,3,2-dioxaborinan-2-yl)-6-methyl-2-pyridinecarbonitrile D67 (154 mg) was dissolved 1,4-dioxane (3 ml) under nitrogen into an 8 ml-vial, then 2-chloro-4-methylpyrimidine (120 mg, 0.937 mmol), cesium fluoride (204 mg, 1.343 mmol), Pd(Ph₃P)₄ (37 mg, 0.032 mmol) and copper(I) iodide (22 mg, 0.116 mmol) were added in sequence. The white solid on the bottom of the vial was crumbled by ultra-sound action for 30 seconds and then the gray slurry was stirred at 70° C., after 1 hour the mixture was stirred again at 70° C. for 30 minutes then the mixture was stored into the freezer overnight. The mixture was diluted with ACN (1 ml), filtered and loaded onto an SCX-10 g column and the column was eluted. After evaporation at reduced pressure of the ammoniacal solution it was obtained the crude target material as pale brown oil (123 mg). This material was purified by Biotage (Snap-25 g silica gel column, EtOAc/Cy from 20:80 to 100% EtOAc) it was obtained the desired target cyano-derivative as pale orange oil (103 mg). This material was dissolved in EtOH (1.2 ml) into an 8 ml-capped vial and a solution of NaOH (187 mg, 4.69 mmol) in water (0.8 ml) was added in one portion. The mixture was stirred 2 hours at 100° C. The solvent was evaporated at reduced pressure and the residue was taken up in water (0.5 ml) and adjusted to pH=2 with 1 M HCl solution. The so obtained solution was loaded onto a pre-conditioned C18 column (25 g, eluted with water and then ACN) to give the title compound D100 (85 mg) as pale yellow solid. UPLC (Acid IPQC): rt=0.36 minutes, peak observed: 230 (M+1). C₁₂H₁₁N₃O₂ requires 229. ¹H NMR (400 MHz, DMSO-d₆) δ ppm 2.56 (s, 3H) 3.32 (s, 3H) 7.37 (d, 1H) 7.49 (d, 1H) 8.37 (d, 1H) 8.73 (d, 1H) 13.05 (br. s., 1H)

### Description 101

### 6-methyl-3,3′-bipyridine-2-carboxylic acid (D101)

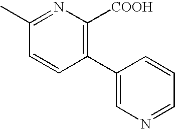

3-(5,5-dimethyl-1,3,2-dioxaborinan-2-yl)-6-methyl-2-pyridinecarbonitrile D67 (154 mg) was dissolved 1,4-dioxane (3 ml) under nitrogen into an 8 ml-vial, then 3-iodopyridine (192 mg, 0.937 mmol), cesium fluoride (204 mg, 1.343 mmol), Pd(Ph₃P)₄ (37 mg, 0.032 mmol) and copper(I) iodide (22 mg, 0.116 mmol) were added in sequence.

The vial was then capped, the white solid on the bottom of the vial was crumbled by ultra-sound action for 30 seconds and then the gray slurry was stirred at 65° C. after 1 hour the mixture was stirred again at 70° C. for 30 minutes to push the reaction to completion, then the mixture was stored into the freezer overnight. The mixture was diluted with ACN (1 ml), filtered and loaded onto an SCX-10 g column, (eluted with ACN then MeOH, with NH₃ 2 M in MeOH). It was obtained the crude target material as pale brown solid (125 mg). This material was purified by Biotage (Snap-25 g silica gel column, EtOAc/Cy from 20:80 to 80:10) it was obtained the desired cyano-derivative as white solid (100 mg). This material was suspended in EtOH (1.2 ml) into an 8 ml-capped vial and a solution of NaOH (187 mg, 4.69 mmol) in water (0.6 ml) was added in one portion. The mixture was stirred 3.5 hours at 100° C.: almost complete conversion into the desired acid. The solvent was evaporated at reduced pressure and the residue was taken up in water (0.5 ml) and adjusted to pH=2 with 1 M HCl solution. The so obtained solution was loaded onto a pre-conditioned C18 column (25 g, eluted with water and then ACN) to give the title compound D101 (81 mg) as white solid. UPLC (Basic GEN_QC): rt=0.33 minutes, peak observed: 215 (M+1). C₁₂H₁₀N₂O₂ requires 214. ¹H NMR (400 MHz, DMSO-d₆) δ ppm 2.57 (s, 3H) 7.45-7.56 (m, 2H) 7.78-7.90 (m, 2H) 8.59 (td, 1.64 Hz, 2H) 13.30 (br. s., 1H)

### Description 102

### methyl 6-methyl-3-(1H-1,2,4-triazol-1-yl)-2-pyridinecarboxylate (D102)

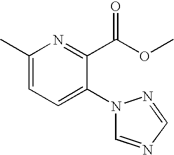

DMF (1.5 ml) was added to a mixture of methyl 3-iodo-6-methyl-2-pyridinecarboxylate D44 (200 mg), 1H-1,2,4-triazole (100 mg, 1.444 mmol), (1R,2R)—N,N′-dimethyl-1,2-cyclohexanediamine (21 mg, 0.148 mmol), bis(copper(I) trifluoromethanesulfonate)-benzene complex (19 mg, 0.038 mmol) and cesium carbonate (470 mg, 1.444 mmol) in a screw-topped vial. The mixture was degassed via 3 vacuum/nitrogen cycles and heated with shaking to 120° C. for 1 hour. The mixture was stirred again at 120° C. for 30 minutes to push the reaction to completion, then the mixture was stored into the freezer overnight. The residue was dissolved/suspended in water/MeOH (1:1, 2 ml) and acidified to pH=2 by addition of 6 M HCl solution. The resulting mixture was evaporated to dryness under reduced pressure and the residue was stored into the freezer overnight. The residue was then triturated with DCM/MeOH (3:1, 10 ml). The mixture was filtered washing with more DCM/MeOH (3:1, 5 ml). The filtrate was treated with trimethylsilyldiazomethane solution (2 M in hexanes, 2 ml, 4 mmol) to re-esterify the acid: after this addition the mixture was stirred at room temperature for 1.5 hours. The reaction mixture was evaporated under reduced pressure and the residue (129 mg, pale brown solid) was purified via Biotage (Snap-25 g silica gel column, AcOEt/Cy from 20:80 to 90:10) it was obtained the title compound D102 (95 mg) as white solid. UPLC (Basic GEN_C): rt=0.44 minutes, peak observed: 219 (M+1). C₁₀H₁₀N₄O₂ requires 218. ¹H NMR (500 MHz, CDCl₃) δ ppm 2.73 (s, 3H) 3.87 (s, 3H) 7.48 (d, 1H) 7.80 (d, 1H) 8.13 (s, 1H) 8.42 (s, 1H)

### Description 103

### 6-methyl-3-(1H-1,2,4-triazol-1-yl)-2-pyridinecarboxylic acid (D104)

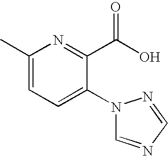

methyl 6-methyl-3-(1H-1,2,4-triazol-1-yl)-2-pyridinecarboxylate D102 (94.2 mg) was dissolved in MeOH (1.4 ml) into a capped vial, then a solution of LiOH (16 mg, 0.668 mmol) in water (0.6 ml) was added in one portion. The mixture was then stirred at room temperature for 90 minutes. The solvent was evaporated at reduced pressure, obtaining the desired target acid as LiOH salt. This material was taken up in water (0.5 ml) and adjusted to pH=2 with 1 M HCl solution and then the so obtained solution loaded onto a pre-conditioned C18 column (25 g, was eluted with water and then acetonitrile) to give the title compound D103 (88 mg) as white solid. UPLC (Basic GEN_C): rt=0.44 minutes, peak observed: 219 (M+1). C₉H₈N₄O₂ requires 218. ¹H NMR (400 MHz, DMSO-d₆) δ ppm 2.59 (s, 3H) 7.62 (d, 1H) 8.07 (d, 1H) 8.23 (s, 1H) 8.99 (s, 1H) 13.50 (br. s., 1H)

### Description 104

### 6-methyl-4-(4,4,5,5-tetramethyl-1,3,2-dioxaborolan-2-yl)-2-pyridinecarbonitrile (D104)

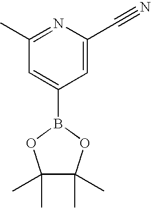

4,4′-bis(1,1-dimethylethyl)-2,2′-bipyridine (8.1 mg, 0.030 mmol) and [Ir(OMe)(COD)]2 (10 mg, 0.015 mmol) were dissolved in THF (3 ml) under nitrogen into a capped vial, then 4,4,5,5-tetramethyl-1,3,2-dioxaborolane (0.3 ml, 2.068 mmol) was dropped into the solution, which became darker, then less dark over 30 seconds. 6-methyl-2-pyridinecarbonitrile (120 mg, 1.016 mmol) was added in one portion (moderate gas evolution) and the mixture became darker. The so obtained dark red/violet solution was stirred at room temperature. After 24 hours the conversion was almost complete. At this point the reaction mixture was left still at room temperature for 48 days. Then it was partitioned between a 10% water solution of KH₂PO₄ (15 ml) and DCM (10 ml), the water layer extracted with DCM and all the organic fractions were joined together, dried over Na₂SO₄ and evaporated at reduced pressure, obtaining the crude boronate title compound (235 mg, orange sticky oil). To this material Et₂O (1 ml) was added, followed by Cy (7 ml): this addition caused the formation of a light orange solid, which was filtered off. The liquid was then evaporated at reduced pressure obtaining a batch of crude title compound D104 (224 mg) orange sticky solid. UPLC (Basic GEN_QC): rt=0.43 minutes, peak observed: 245 (M+1). C₁₃H₁₇BN₂O₂ requires 244.

¹H NMR (400 MHz, DMSO-d₆) δ ppm 1.32 (s, 12H) 2.56 (s, 3H) 7.78 (s, 1H) 7.86 (s, 1H)

### Description D105

### 6-methyl-4-(2-pyrimidinyl)-2-pyridinecarboxylic acid (D105)

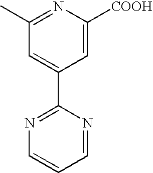

6-methyl-4-(4,4,5,5-tetramethyl-1,3,2-dioxaborolan-2-yl)-2-pyridinecarbonitrile D104 (221 mg) was dissolved 1,4-dioxane (5 ml) under nitrogen into an 8 ml-vial, then 2-bromopyrimidine (173 mg, 1.086 mmol), cesium fluoride (275 mg, 1.811 mmol), Pd(Ph₃P)₄ (60 mg, 0.052 mmol) and copper(I) iodide (25 mg, 0.131 mmol) were added in sequence. The vial was then capped and stirred at 65° C. After 3 hours the mixture was stirred at 80° C. for 19 hours. New Pd(Ph₃P)₄ (80 mg, 0.069 mmol), 2-bromopyrimidine (100 mg, 0.629 mmol) and K₂CO₃ (200 mg, 1.447 mmol) were added to the mixture which was stirred at 100° C. for 19 hours: the mixture was cooled to room temperature. The mixture was partitioned between water (30 ml) and Et₂O (30 ml). The water phase was extracted with Et₂O; all the organic fractions were joined together, washed with brine, dried over Na₂SO₄, filtered and evaporated at reduced pressure, obtained the crude target cyano derivative as orange oil (366 mg). This material was purified by Biotage (Snap-50 g silica gel column, from pure cyclohexane to AcOEt/cyclohexane 50:50). It was obtained the desired intermediate as pale yellow solid (56.5 mg). All this material was dissolved in EtOH (0.7 ml) into a capped 8 ml-vial, then a solution of NaOH (35 mg, 0.875 mmol) in water (0.3 ml) was added in one portion and the resulting mixture was stirred at 100° C. after 3 hours the reaction was almost complete. The solvent was evaporated at reduced pressure and the residue dried under vacuum at 45° C. for 3 hours, obtaining the desired acid as sodium salt, but containing an excess of NaOH. This material was taken up in water (0.5 ml) and adjusted to pH=2 with 1 M HCl solution. The so obtained solution was loaded onto a pre-conditioned C18 column (25 g, eluted with water and then ACN) to give the title compound D105 (61 mg) as white solid. UPLC (Acid IPQC): rt=0.39 minutes, peak observed: 216 (M+1). C₁₁H₉N₃O₂ requires 215.

### Description D106

### 3-(2-pyrimidinyl)-2-pyridinecarboxylic acid (D106)

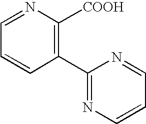

2-(tributylstannanyl)pyrimidine (445 mg, 1.206 mmol) was dissolved in 1,4-dioxane (2 ml). To the stirred solution 3-bromo-2-pyridinecarbonitrile (200 mg, 1.093 mmol) was added dissolved in 1,4-dioxane (2 ml), followed by Pd(Ph₃P)₄ (125 mg, 0.108 mmol).

The mixture was heated by microwave irradiation at 160° C. for 60 minutes. The solvent was removed at reduced pressure and the dark brown residue partitioned between water (30 ml) and Et₂O (30 ml). The water phase was extracted with Et₂O; all the organic fractions were joined together, dried over Na₂SO₄, filtered and evaporated at reduced pressure, obtaining a gray solid (719 mg). This material was purified by Biotage (Snap-50 g silica gel column, from pure Cy to AcOEt/Cy 50:50). After evaporation at reduced pressure of the pure collected fractions it was obtained the desired cyano derivative as white solid (114.7 mg). This material was dissolved in EtOH (2 ml) into an 8 ml-capped vial and a solution of NaOH (79 mg, 1.975 mmol) in water (1 ml) was added in one portion. The resulting mixture was stirred for 5 hours at 100° C. 14%-UV of primary amide was still present, so new NaOH (11 mg, 0.275 mmol) was added. The resulting mixture was stirred for other 2 hours at 100° C. The solvent was evaporated at reduced pressure, obtaining the desired acid as sodium salt. This material was taken up in water (0.5 ml) and adjusted to pH=2 with 1 M HCl solution. The so obtained solution was loaded onto a pre-conditioned C18 column (25 g, eluted with water and then ACN.) to give the title compound D106 (116 mg) as white solid. UPLC (Basic GEN_QC): rt1=0.17 minutes and rt2=0.24 minutes, peak observed: 202 (M+1). C₁₀H₇N₃O₂ requires 201. ¹H NMR (400 MHz, DMSO-d₆) δ ppm 7.54 (t, 1H) 7.67 (dd, 1H) 8.47 (dd, 1H) 8.71 (dd, 1H) 8.94 (d, 2H) 13.16 (br. s., 1H).

### Description 107

### 2-(5-methyl-2-pyridinyl)pyrimidine (D107)

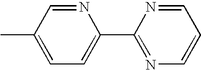

A solution of n-butyl lithium (7.4 ml, 18.53 mol of a 2.5 M solution in hexane) was added dropwise to a solution of 2-bromo-5-methylpyridine (3 g, 17.44 mmol) in degassed THF (45 ml) at −78° C. under a nitrogen atmosphere. The mixture was stirred for 0.5 hours at −78° C. after the addition was complete. A solution of zinc chloride (52.32 ml, 52.32 mmol) was added dropwise, so that the temperature was kept below −60° C. A precipitate formed and the solution was stirred at −78° C. for a further 0.5 hours. Tetrakis(triphenylphosphine)palladium(0) (1.04 g, 0.9 mmol) was added followed by 2-bromopyrimidine (1.98 g, 12.45 mmol) in degassed THF (45 ml). After complete addition, the reaction mixture was refluxed for 8 hours. The reaction mixture cooled down to room temperature, and few ml of methanol were added to quench traces of Bu—Li. The solid obtained was filtered off and washed with THF. The solid was triturated with water for 1 hour, filtered and the aqueous fraction collected and basified with saturated aqueous carbonate and extracted with DCM. The organic fractions were collected, dried over MgSO₄, filtered and the solvent removed by rotary evaporation affording the title compound D107 (0.862 g) as a yellow solid.

MS: (ES/+) m/z: 171.8 (M+1). C₁₀H₉N₃ requires 171.

### Description 108

### 2-(5-methyl-1-oxido-2-pyridinyl)pyrimidine (D108)

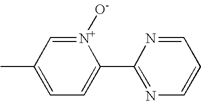

2-(5-methyl-2-pyridinyl)pyrimidine D107 (1.096 g) was dissolved in DCM (100 ml) and 3-chloroperoxybenzoic acid 70% (1.89 g, 7.70 mmol) added in small portions. The final mixture was stirred at room temperature overnight. Next day, the mixture of reaction was extracted with an aqueous solution of bicarbonate (2×50 ml). The organic fraction was taken and dried with MgSO₄, filtered and the solvent evaporated. Crude title compound was obtained as a solid (2.911 g) that was chromatographed over silica gel (using AcOEt/MeOH from 100/0 to 80/20 as eluent) affording the title compound D108 (0.436 g).

MS: (ES/+) m/z: 188.2 (M+1). C₁₀H₉N₃O requires 187. ¹H NMR (400 MHz, CDCl₃) δ ppm 2.35 (s, 3H) 7.15 (d, 1H) 7.35 (d, 1H) 7.55 (d, 1H) 8.2 (s, 1H) 8.9 (d, 2H).

### Description 109

### 3-methyl-6-(2-pyrimidinyl)-2-pyridinecarbonitrile (D109)

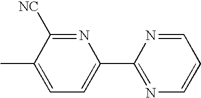

2-(5-methyl-1-oxido-2-pyridinyl)pyrimidine D108 (416 mg) was dissolved in nitromethane (7.32 ml) and trimethylsilylcyanide added (1.17 ml, 9.32 mmol) followed by N,N-dimethylcarbamoyl chloride (1.03 g, 9.55 mmol). The final mixture was stirred at room temperature. After 4 days the solvent was evaporated and the crude obtained was subjected to column chromatography (using DCM/MeOH from 100/0 to 99/1 as eluent mixture). The desired title compound D109 (0.316 g) was obtained as yellow oil.

MS: (ES/+) m/z: 197.1 (M+1). C₁₁H₈N₄ requires 196.

### Description (D110)

### 3-methyl-6-(2-pyrimidinyl)-2-pyridinecarboxylic acid HCl salt. (D110)

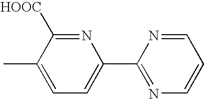

3-methyl-6-(2-pyrimidinyl)-2-pyridinecarbonitrile D109 (0.05 g) was placed into a sealed tube and dissolved in an aqueous solution 6 N of hydrochloric acid (3 ml). The tube was heated at 110° C. and stirred for 17 hours. The rest of the material D109 (0.266 g) were added to the mixture and also more hydrochloric acid 6 N solution (32 ml). The total amount of solution was divided in two sealed tubes. The mixture was heated at 110° C. overnight. The reaction mixture was left to react for the weekend (72 hours). The solvent was evaporated and dried in the high vacuum oven at 40° C. overnight. Title compound D110 (0.42 g) was obtained as pale yellow solid.

MS: (ES/+) m/z: 216.2 (M+1). C₁₁H₉N₃O₂ requires 215. ¹H NMR (400 MHz, MeOD) δ ppm 2.8 (s, 3H) 7.85 (m, 1H) 8.25 (m, 1H) 8.8 (m, 1H) 9.2 (m, 2H).

### Description 111

### 2-chloro-6-{[((1R,4S,6R)-3-{[6-methyl-3-(2-pyrimidinyl)-2-pyridinyl]carbonyl}-3-azabicyclo[4.1.0]hept-4-yl)methyl]amino}-4-(trifluoromethyl)-3-pyridinecarbonitrile (D111)

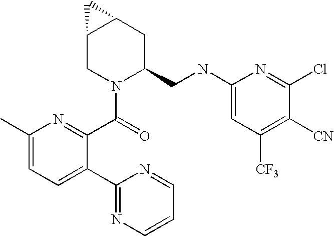

[((1R,4S,6R)-3-{[6-methyl-3-(2-pyrimidinyl)-2-pyridinyl]carbonyl}-3-azabicyclo[4.1.0]hept-4-yl)methyl]amine D25 (50 mg), 2,6-dichloro-4-(trifluoromethyl)-3-pyridinecarbonitrile (37.3 mg, 0.155 mmol), DIPEA (0.054 ml, 0.309 mmol) were collected in DMSO (2 ml) and shaken at 80° C. for 2 hours, then solvent was removed under vacuum, and the resulting crude was purified on a Biotage SP1 over a 50 g C18 SNAP column (with a gradient of ACN and water, modified with 0.5% HCOOH). Fractions containing the product were collected and neutralised with a 2 g SCX column (washing with MeOH and eluting with 2 M ammonia in MeOH) to give the title compound D111 (31 mg) and a second batch with lower purity (30 mg) which was further purified (Biotage SP1, over a column stacking of 2×4 g Analogix column, eluting with a gradient of DCM and MeOH) to give the title compound D111 (17 mg). UPLC (Acid GEN_QC SS): rt1=0.93 minutes and rt2=0.95 (rotamers present), peaks observed: 528 (M+1). C₂₅H₂₁ClF₃N₇O requires 527.

### Description 112

### {6-methyl-3-[(1-methylethyl)oxy]-2-pyridinyl}methanol (D112)

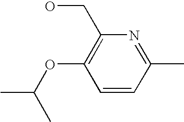

2-(hydroxymethyl)-6-methyl-3-pyridinol (1.5 g, 10.78 mmol), K₂CO₃ (7.45 g, 53.9 mmol) and 2-bromopropane (2.040 ml, 21.56 mmol) were dissolved in DMF (15 ml). The mixture was left stirring at room temperature overnight, was transferred into a separatory funnel containing 150 ml of water and was extracted with EtOAc. The organic phase was washed with water and then dried and evaporated to give the title compound D112 (1.85 g) submitted to the next step without further purification. MS: (ES/+) m/z: 182 (M+1). C₁₀H₁₅NO₂ requires 181. ¹H-NMR (400 MHz, CDCl₃) δ ppm: 7.07 (d, 1H), 7.00 (d, 1H), 4.70 (s, 2H), 4.46-4.56 (m, 2H), 2.50 (s, 3H), 1.35 (s, 3H), 1.34 (s, 3H).

### Description 113

### 6-methyl-3-[(1-methylethyl)oxy]-2-pyridinecarboxylic acid (D113)

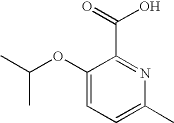

To a solution of {6-methyl-3-[(1-methylethyl)oxy]-2-pyridinyl}methanol D112 (1.85 g) in acetonitrile (50 ml) and phosphate buffer (38.0 ml) was added TEMPO (0.223 g, 1.429 mmol) at room temperature. After warming to 35° C. a solution of NaClO₂ (4.62 g, 51.0 mmol) in water (10 ml) and a solution of NaClO (19.39 ml, 40.8 mmol) were added simultaneously over 1 hour. After stirring 4 hours at 35° C., water (40 ml) was added to the reaction mixture which was then adjusted to pH=8 by addition of 1 M NaOH. The mixture was poured into ice-cold saturated aqueous sodium thiosulfate solution (100 ml) and stirring was continued for 30 minutes. The pH was adjusted to pH=3 by slow addition of 1 M HCl and the aqueous phase was extracted with DCM. The combined organic layers were washed with brine, dried over Na₂SO₄ and concentrated to afford the title compound D113 (1.46 g). MS: (ES/+) m/z: 196 (M+1). C₁₀H₁₃NO₃ requires 195. ¹H-NMR (400 MHz, DMSO d₆) δ ppm: 12.90 (bs, 1H), 7.49 (d, 1H), 7.29 (d, 1H), 4.61 (m, 1H), 2.39 (s, 3H), 1.24 (d, 6H).

### Description 114

### methyl 6-methyl-3-[(trimethylsilyl)ethynyl]-2-pyridinecarboxylate (D114)

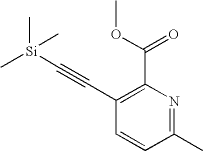

In a 10 ml round bottom flask methyl 3-iodo-6-methyl-2-pyridinecarboxylate D44 (200 mg), bis(triphenylphosphine)palladium(II) chloride (86 mg, 0.123 mmol), CuI (23.37 mg, 0.123 mmol) and DIPEA (0.391 mL, 2.238 mmol) were dissolved in DMF (2 ml) and then degassed. To this solution trimethylsilylacetylene (0.111 ml, 0.794 mmol) was added dropwise. After 30 min stirring at 23° C. water (2 ml) was added and extracted with EtOAc, the collected organic layer was dried (Na₂SO₄), filtered and evaporated under reduced pressure giving a brown oil which was purified by column chromatography on silica gel (SNAP KP-Sil 10 g; eluted with Cy/EtOAc 15 CV from 1/0 to 8/2) to give the title compound D114 (178 mg) as brown oil. UPLC (Basic GEN_C): rt=0.92 minutes. peaks observed: 248 (M+1). C₁₃H₁₇NO₂Si requires 247. ¹H NMR (400 MHz, DMSO-d₆) δ ppm 7.92 (d, 1H), 7.46 (d, 1H), 3.88 (s, 3H), 0.10-0.34 (m, 9H).

### Description 115

### methyl 3-ethynyl-6-methyl-2-pyridinecarboxylate (D115)

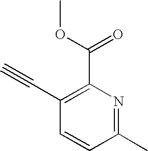

In a 25 ml round bottom flask methyl 6-methyl-3-[(trimethylsilyl)ethynyl]-2-pyridinecarboxylate D114 (178 mg) was dissolved in THF (4.8 ml) and treated with TBAF (1 M in THF) (0.935 ml, 0.935 mmol) at 0° C. The mixture was stirred for 15 minutes, then NaHCO₃ aqueous saturated solution (6 ml) and EtOAc (10 ml) were added. After the separation, the organic phase was washed with NaHCO₃ aqueous saturated solution. The collected aqueous layers were backextracted with EtOAc and the organic layers were joined together with the first EtOAc, dried (Na₂SO₄), filtered and evaporated under reduced pressure. The black oil obtained was purified by silica gel chromatography (SNAP KP-Sil 10 g cartridge; eluted with Cy/EtOAc 15 CV from 1/0 to 8/2). Collected and evaporated fractions gave the title compound D115 (83 mg) as solid.

UPLC (Basic GEN_C): rt=0.57 minutes. peaks observed: 176 (M+1). C₁₀H₉NO₂ requires 175. ¹H NMR (400 MHz, DMSO-d₆) δ ppm 7.96 (d, 1H), 7.49 (d, 1H), 4.55 (s, 1H), 3.32 (s, 3H), 2.55 (s, 3H).

### Description 116

### methyl 6-methyl-3-(3-methyl-5-isoxazolyl)-2-pyridinecarboxylate (D1161

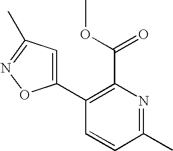

A solution of (1Z)-N-hydroxyethanimidoyl chloride (77 mg, 0.822 mmol) in toluene (2.2 ml) was cooled to 0° C. and methyl 3-ethynyl-6-methyl-2-pyridinecarboxylate D115 (60 mg) was added followed by TEA (0.119 ml, 0.856 mmol). The resulting mixture was stirred for 1 hour at 130° C. EtOAc (10 ml) and NH₄Cl aqueous saturated solution (5 ml) were added and after the separation the aqueous phase was extracted with EtOAc. Collected organic layers were dried (Na₂SO₄), filtered and evaporated under reduced pressure to give a brown solid which was purified by silica gel chromatography (SNAP KP-Sil 25 g; eluted with Cy/EtOAc from 1:0 to 6:4). Collected fractions gave the title compound D116 (74 mg) as white solid. UPLC (Basic GEN_C): rt=0.62 minutes. peaks observed: 233 (M+1). C₁₀H₉NO₂ requires 232. ¹H NMR (500 MHz, DMSO-d₆) δ ppm 8.16 (d, 1H), 7.60 (s, 1H), 6.74 (s, 1H), 3.85 (s, 3H), 2.56 (s, 3H), 2.29 (s, 3H).

### Description 117

### 6-methyl-3-(3-methyl-5-isoxazolyl)-2-pyridinecarboxylate lithium salt (D117)

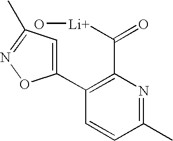

To a solution of methyl 6-methyl-3-(3-methyl-5-isoxazolyl)-2-pyridinecarboxylate D116 (74 mg) in EtOH (3.5 ml) and water (0.875 ml) was added LiOH (9.92 mg, 0.414 mmol) and the resulting mixture was stirred at 23° C. After 6.5 hours the solvents were removed under reduced pressure to give a white solid the title compound D117 (86 mg). UPLC (Basic GEN_C): rt=0.33 minutes. peaks observed: 219 (M+1). C₁₁H₉N₂O₃₋.Li⁺ requires 218. ¹H NMR (400 MHz, DMSO-d₆) δ ppm 7.90 (d, 1H), 7.12 (d, 1H), 6.80 (s, 1H), 2.44 (s, 3H), 2.26 (s, 3H).

### Description 118

### 6-methyl-3-(4-methyl-1H-imidazol-1-yl)-2-pyridinecarboxylic acid (D118)

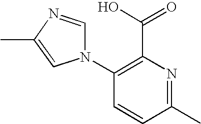

Copper(I) iodide (2.3 mg, 0.012 mmol), 1,10-phenanthroline (2 mg, 0.011 mmol), cesium carbonate (67 mg, 0.206 mmol), 4-methyl-1H-imidazole (9.8 mg, 0.119 mmol) and methyl 3-iodo-6-methyl-2-pyridinecarboxylate D44 (27.5 mg) were mixed together into a microwave vial; DMF (1 ml) was added and the mixture stirred at room temperature for 15 minutes: no reaction. The mixture was then heated by microwave irradiation at 100° C. for 30 minutes. The mixture was then heated by microwave irradiation at 100° C. for a further 2 hours. New copper(I) iodide (12 mg, 0.063 mmol), 1,10-phenanthroline (10 mg, 0.055 mmol), cesium carbonate (67 mg, 0.206 mmol), 4-methyl-1H-imidazole (9.8 mg, 0.119 mmol) were added, followed by DMF (1 ml) and the mixture heated by microwave irradiation at 100° C. for 1 hour. The mixture was diluted with DCM (2 ml). A second reaction was carried out: reacting copper(I) iodide (2.3 mg, 0.012 mmol), 4,7-bis(methyloxy)-1,10-phenanthroline (2.8 mg, 0.012 mmol), cesium carbonate (67 mg, 0.206 mmol), 4-methyl-1H-imidazole (9.8 mg, 0.119 mmol) and methyl 3-iodo-6-methyl-2-pyridinecarboxylate D44 (27.5 mg) mixed together in DMF (1 ml). The solutions of these two reaction mixtures were combined to obtain a new mixture which was filtered and loaded onto an SCX-5 g column and the column was eluted. After evaporation of the ammonia solution it was obtained the crude acid as orange foam (45 mg); 44 mg were purified by a preparative HPLC (CUSTOM_Prep_Purification). After evaporation at reduced pressure of the preparative HPLC solution it was obtained the title compound D118 (14.4 mg) as white solid. UPLC (Basic GEN_QC): rt=0.33 minutes. peaks observed: 218 (M+1). C₁₁H₁₁N₃O₂ requires 217. ¹H NMR (500 MHz, DMSO-d₆) δ ppm 13.42 (br. s., 1H), 7.78 (d, 1H), 7.71 (s, 1H), 7.44 (d, 1H), 7.09 (s, 1H), 2.52 (s, 3H), 2.13 (s, 3H).

### Description 119

### 4(5)-Fluoro-1H-imidazole (D119)

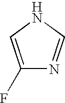

A 1.6 M solution of butyllithium in hexanes (37.5 ml, 59.9 mmol) was added dropwise to a stirred solution of N,N-dimethyl-1H-imidazole-1-sulfonamide (10 g, 57.1 mmol) in THF (60 ml) at −78° C. The reaction was stirred for 20 minutes then a solution of TBDMSCl (8.60 g, 57.1 mmol) in THF (30 ml) was added dropwise at the same temperature. The reaction was allowed to warm gradually to room temperature and stirred overnight. The reaction mixture was cooled to −78° C. and a 1.6 M solution of butyllithium in hexanes (37.5 ml, 59.9 mmol) was added. The reaction was stirred for 1 hour then a solution of N-fluorobenzenesulfonimide (18.00 g, 57.1 mmol) in THF (50 ml) was added. The reaction was stirred at −78° C. for 1 hour then allowed to warm to room temperature and stirred for another 1 hour. The reaction was quenched with 1M HCl solution (100 ml) and stirred for 1 hour. The THF was evaporated under reduced pressure then the aqueous phase was washed with EtOAc (2×200 ml), back-extracting each EtOAc wash with HCl (2M, 100 ml). The combined acidic aqueous phases were adjusted to pH=9 with NaOH pellets and the aqueous phase was extracted with EtOAc (8×200 ml). The organic phases were dried over Na₂SO₄ and evaporated under reduced pressure. The crude residue was treated with a 1M solution of TBAF in THF (30 ml, 30.0 mmol) and heated to 60° C. for 2 hours. The reaction mixture was divided in two and each half was loaded onto a pre-conditioned SCX cartridge (70 g) and the cartridge was eluted. The basic fractions from both columns were combined and evaporated under reduced pressure. The residue was purified by flash chromatography on silica gel (340 g, eluting with a gradient of EtOAc in Cy from 50 to 100%) and then by treating with activated charcoal in EtOAc for 15 minutes to afford the title compound D119 (3.16 g) as a yellow solid; MS: (ES/+) m/z: 87 (M+1); ¹H NMR (CDCl₃): δ 6.56 (d, 1H), 7.26 (s, 1H), 9.55 (t, 1H); ¹⁹F NMR (CDCl₃): δ 138.0.

### Description D120

### 3-(4-fluoro-1H-imidazol-1-yl)-6-methyl-2-pyridinecarboxylic acid (D120)

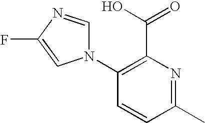

NMP (1.5 ml) was added to a mixture of 4-fluoro-1H-imidazole D119 (23.30 mg), methyl 3-iodo-6-methyl-2-pyridinecarboxylate D44 (50 mg), 4,7-bis(methyloxy)-1,10-phenanthroline (6.50 mg, 0.027 mmol), bis(copper(I) trifluoromethanesulfonate), benzene complex (4.54 mg, 9.02 μmol) and cesium carbonate (94 mg, 0.289 mmol) in a screw-topped vial with septum and the mixture was rapidly degassed via three vacuum/nitrogen cycles. The reaction mixture was then shaken and heated to 110° C. for 3 hours. The reaction was left at room temperature for 48 hours and then loaded onto a pre-conditioned SCX cartridge (5 g). The cartridge was eluted. By UPLC the NH₃ in MeOH fractions contained the de-iodinated side product but no desired product however another peak corresponding to the acid of the desired product was present. By UPLC the MeOH fractions were seen to contain mainly NMP but also a very small quantity of the desired ester product and also some of the acid of the desired product. The MeOH fractions were evaporated under reduced pressure (NMP was not removed) and the residue was treated with KOH (5 M, 5 ml) for 5 minutes then diluted with water (10 ml) and washed with DCM to remove NMP. The aqueous phase was neutralised then evaporated to dryness under reduced pressure and loaded onto a C18 cartridge. This was eluted with water then with MeOH to recover some acid of the desired product. These fractions were combined with the NH₃/MeOH fractions from the SCX cartridge and evaporated under reduced pressure. The residue was chromatographed on the Biotage (mobile phase A was water made up with 0.1% formic acid, mobile phase B was acetonitrile made up with 0.1% formic acid. 12 M C18 column was eluted with phase A for 3 column volumes then in gradient 0-20% A/B). The fractions containing the acid of the desired product were partially evaporated under reduced pressure then loaded onto a pre-conditioned SCX cartridge (2 g) to give a mixture of the title compound D120 (15 mg), of unreacted 4-fluoro-1H-imidazole and of NMP, as off white solid which was used as such without further purification in the next reaction.

UPLC: (Acid QC_POS_{—}70_{—}900): peak observed: 475 (M+1). C₂₃H₂₂F₄N₆O requires 474.

Rt1=0.23 min is unreacted 4-fluoro-1H-imidazole

Rt2=0.33 min is NMP

Rt3=0.36 min is product D120 peak observed: 222 (M+1). C₁₀H₈FN₃O₂ requires 221.

### Description D121

### 6-methyl-3-[4-(trifluoromethyl)-1H-imidazol-1-yl]-2-pyridinecarboxylic acid (D121)

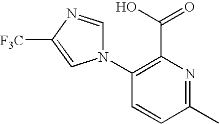

NMP (1.5 ml) was added to a mixture of 4-trifluoromethyl-1H-imidazole (65.8 mg, 0.484 mmol), methyl 3-iodo-6-methyl-2-pyridinecarboxylate D44 (67 mg), 4,7-bis(methyloxy)-1,10-phenanthroline (8.72 mg, 0.036 mmol), bis(copper(I) trifluoromethanesulfonate), benzene complex (6.09 mg, 0.012 mmol) and cesium carbonate (126 mg, 0.387 mmol) in a screw-topped vial with septum and the mixture was rapidly degassed via three vacuum/nitrogen cycles. The reaction mixture was then shaken and heated to 90° C. for 2 hours. The reaction mixture was heated to 110° C. for 2 hours. Another quantity of bis(copper(I) trifluoromethanesulfonate), benzene complex (6.09 mg, 0.012 mmol) was added and the mixture was heated with shaking to 110° C. for 2 hours. UPLC check shows all the methyl 3-iodo-6-methyl-2-pyridinecarboxylate has reacted but there are still only traces of the expected product methyl 6-methyl-3-[4-(trifluoromethyl)-1H-imidazol-1-yl]-2-pyridinecarboxylate—closer scrutiny showed that signals in the mass spectrum corresponding to the acid 6-methyl-3-[4-(trifluoromethyl)-1H-imidazol-1-yl]-2-pyridinecarboxylic acid co-elute with 4,7-bis(methyloxy)-1,10-phenanthroline in the UPLC. UPLC in basic conditions showed a better separation confirming the formation of the acid 6-methyl-3-[4-(trifluoromethyl)-1H-imidazol-1-yl]-2-pyridinecarboxylic acid as well as de-iodinated product. The reaction mixture was cooled, diluted with water (15 ml) and loaded onto an ENV+ cartridge (1 g). The cartridge was eluted with water and then with MeOH. UPLC check of the water washes indicated they contain NMP as well as the deiodinated product and the excess 4-trifluoromethyl-1H-imidazole. UPLC check of the MeOH washes indicated they contained the acid 6-methyl-3-[4-(trifluoromethyl)-1H-imidazol-1-yl]-2-pyridinecarboxylic acid plus some impurities. The MeOH washes were combined and evaporated under reduced pressure to give a dark brown residue which was purified on the Biotage (mobile phase A was water made up with 0.1% formic acid, mobile phase B was acetonitrile made up with 0.1% formic acid. 12M C18 column was eluted with phase A for 2 column volumes then in gradient 0-50% A/B). The fractions containing the acid of the desired product were not pure by UPLC—they were combined and evaporated under reduced pressure to give 35 mg of a solid residue which was further purified by FractionLynx (Acid LC1, note a considerable quantity of the solid was insoluble in DMSO/MeOH). The fraction containing the desired product was evaporated under reduced pressure to give the title compound D121 (9 mg) of a pale orange glass. UPLC (Basic GEN_QC): rt=0.38 minutes, peak observed: 272 (M+1). C₁₁H₈F₃N₃O₂ requires 271.

### Description D122

### methyl 6-methyl-3-(1,3-thiazol-2-yl)-2-pyridinecarboxylate (D122)

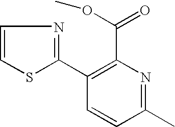

2-(tributylstannanyl)-1,3-thiazole (68 mg, 0.182 mmol) was dissolved in 1,4-dioxane (1 ml). To the stirred solution methyl 3-iodo-6-methyl-2-pyridinecarboxylate D44 (50 mg) was added, followed by Pd(Ph₃P)₄ (20 mg, 0.017 mmol).

The resulting orange solution was heated into a microwave reactor at 120° C. for 30 minutes: complete conversion. The mixture was loaded onto an SCX-5 g column and the column was eluted. It was obtained the crude target material as colorless oil, which was then purified via Biotage (Snap-10 g silica gel column, AcOEt:Cy 25:75). It was obtained the title compound D122 (31.5 mg) as white solid.

UPLC (Basic GEN_QC): rt=0.60 minutes, peak observed: 235 (M+1). C₁₁H₁₀N₂O₂S requires 234.

### Description D123

### lithium 6-methyl-3-(1,3-thiazol-2-yl)-2-pyridinecarboxylate (D123)

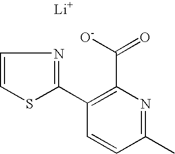

Methyl 6-methyl-3-(1,3-thiazol-2-yl)-2-pyridinecarboxylate D122 (30.2 mg) was dissolved in EtOH (0.7 ml) into a capped vial, then a solution of lithium hydroxide (4.7 mg, 0.196 mmol) in water (0.3 ml) was added in one portion. The mixture was then stirred at room temperature for 90 minutes and the solvent was evaporated at reduced pressure obtaining the title compound D123 as white solid (30.5 mg). UPLC (Basic GEN_QC): rt=0.32 minutes, peak observed: 221 (M+1). C₁₀H₇N₂O₂S. Li⁺ requires 220. ¹H NMR (400 MHz, DMSO-d₆) δ ppm 8.08 (d, 1H), 7.84 (d, 1H), 7.70 (d, 1H), 7.11 (d, 1H), 2.43 (s, 3H).

### Description D124

### 2-chloro-N-(2-hydroxypropyl)-6-methyl-3-pyridinecarboxamide (D124)

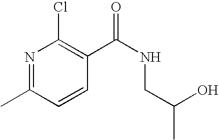

In a 100 ml round bottom flask 2-chloro-6-methyl-3-pyridinecarboxylic acid (1 g, 5.83 mmol) was added and dissolved in DMF (20 ml). To this solution DIPEA (5.09 ml, 29.1 mmol) and TBTU (2.246 g, 6.99 mmol) were added and the mixture stirred at room temperature for 30 minutes. After this time 1-amino-2-propanol (0.876 g, 11.66 mmol) was added and the resulting solution left under stirring at room temperature for 14 hour. After this time the reaction mixture was transferred into a separatory funnel containing brine and extracted with EtOAc. The combined organic phases were dried (Na₂SO₄) and evaporated to give the title compound D124 as crude yellow oil (2.1 g) that was used in the next step without further purification. MS: (ES/+) m/z: 229 (M+1). C₁₀H₁₃ClN₂O₂ requires 228.

### Description D125

### 2-chloro-6-methyl-N-(2-oxopropyl)-3-pyridinecarboxamide (D125)

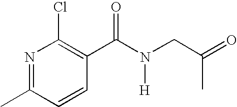

Into a 7 ml capped vial 2-chloro-N-(2-hydroxypropyl)-6-methyl-3-pyridinecarboxamide D124 (1.3 g), DCM (2 ml) and Dess-Martin periodinane (3.13 g, 7.39 mmol) were added and the resulting mixture left under stirring at room temperature for 4 hours. After this time solvent was removed and the crude purified by column chromatography on silica gel (DCM-MeOH=from 100/0 to 50/50). Collected fractions gave the crude title compound D125 (1.1 g) used without further purification. MS: (ES/+) m/z: 227 (M+1). C₁₀H₁₁ClN₂O₂ requires 226.

### Description D126

### 2-chloro-6-methyl-3-(5-methyl-1,3-oxazol-2-yl)pyridine (D126)

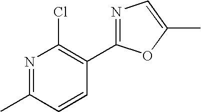

Into a 7 ml screw capped vial 2-chloro-6-methyl-N-(2-oxopropyl)-3-pyridinecarboxamide D125 (1.1 g) was dissolved in THF (2 ml) and Burgess reagent (1.041 g, 4.37 mmol) was added and the reaction mixture stirred at 50° C. for 2 hours. After this time volatiles were removed under vacuum and the crude purified by column chromatography on silica gel (flash master, silica NH₂ cartridge, Cy/EtOAc=from 100/0 to 80/20) to give the title compound D126 (430 mg) as an off-white solid. MS: (ES/+) m/z: 209 (M+1). C₁₀H₉ClN₂O requires 208.

### Description D127

### 2-ethenyl-6-methyl-3-(5-methyl-1,3-oxazol-2-yl)pyridine (D127)

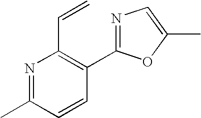

Into a microwave vial 2-chloro-6-methyl-3-(5-methyl-1,3-oxazol-2-yl)pyridine D126 (0.365 g), Pd(Ph₃P)₄ (0.091 g, 0.079 mmol) were added and dissolved in 1,4-dioxane (5 ml). The mixture was degassed and filled with nitrogen, then tributyl(vinyl)tin (0.506 ml, 1.732 mmol) was added and the reaction mixture was stirred at 95° C. for 1.5 hours. The mixture was filtered through a celite pad washed with EtOAc (20 ml), solvent was removed under vacuum to give the title compound D127 (1.15 g) as a dark yellow oil. This material was used in the next step without further purification. UPLC (Basic GEN_QC): rt=0.79 minutes, peak observed: 201 (M+1). C₁₂H₁₂N₂O requires 200.

### Description 128

### 6-methyl-3-(5-methyl-1,3-oxazol-2-yl)-2-pyridinecarbaldehyde (D128)

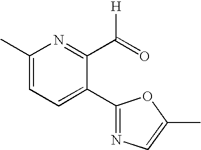

Into a 7 ml screw capped vial 2-ethenyl-6-methyl-3-(5-methyl-1,3-oxazol-2-yl)pyridine D127 (1.15 g), was dissolved in THF (10 ml) and water (15 ml) was added followed by osmium tetroxide 2.5% wt solution in methyl-2-propanol (3.61 ml, 0.287 mmol). After 5 minutes under stirring sodium periodate (1.843 g, 8.61 mmol) was added and the mixture left under stirring at room temperature. The mixture was transferred into a reparatory funnel with EtOAc and brine and the mixture extracted with EtOAc. The combined organic phases were dried (Na₂SO₄) and evaporated under vacuum to give the title compound D128 (0.343 g) as brown crude oil. UPLC (Basic GEN_QC): rt=0.55 minutes, peak observed: 203 (M+1). C₁₁H₁₀N₂O₂ requires 202.

### Description D129

### 6-methyl-3-(5-methyl-1,3-oxazol-2-yl)-2-pyridinecarboxylic acid (D129)

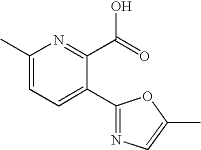

In a 250 ml flask 6-methyl-3-(5-methyl-1,3-oxazol-2-yl)-2-pyridinecarbaldehyde D128 (343 mg) was dissolved in THF (3.50 ml) and water (7 ml), to the mixture sodium hydroxide (67.8 mg, 1.696 mmol) and potassium permanganate (536 mg, 3.39 mmol) were added and stirred at room temperature for 5 min. The organic solvent was removed under vacuum and the residue was filtered on a celite pad, washed with aq 1M HCl. The aqueous layer was charged on Varian C18 column (50 g, washed with 5 CV of water and eluted with 1 CV of MeOH) to give a yellow oil, (126 mg). It was purified by chromatography on silica gel (KP-Sil 25 g column; DCM/MeOH/AcOH 94/4/2). To give a colorless vitreous solid which was triturated with Et₂O (1 ml) yielding, the title compound D129 (30 mg) as white solid. MS (ES−) peak observed 217 (M−1), C₁₁H₁₀N₂O₃ requires 218. UPLC walkup rt=4.40 minutes.

### Description 130

### 3-[(phenylmethyl)amino]-2-butanol (D130)

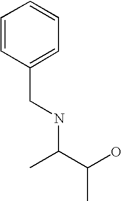

3-hydroxy-2-butanone (2 g, 22.70 mmol) and (phenylmethyl)amine (2.432 g) were dissolved together in DCM (50 ml), then acetic acid (6.50 ml, 114 mmol) and sodium triacetoxyborohydride (5.77 g, 27.2 mmol) were added and the reaction was stirred overnight at room temperature 100 ml of NaHCO₃ saturated solution were added and the product was extracted with DCM. All the organic layers were combined together, dried over Na₂SO₄ anhydrous, filtered and concentrated to give a crude product which was purified by SCX chromatography (Column size 50 g). It was recovered the title compound D130 (4 g). UPLC: (Basic Gen_QC): rt=0.60 minutes, peak observed: 180 (M+1). C₁₁H₁₇NO requires 179.

### Description 131

### (2-{[(1,1-dimethylethyl)(diphenyl)silyl]oxy}-1-methylpropyl)(phenylmethyl)amine (D131)

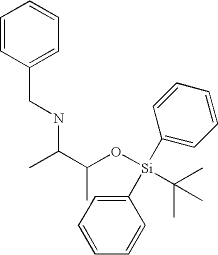

3-[(phenylmethyl)amino]-2-butanol D130 (4 g) was dissolved in DMF (50 ml) then imidazole (4.56 g, 66.9 mmol) and chloro(1,1-dimethylethyl)diphenylsilane (6.13 g, 22.31 mmol) were added and the reaction was stirred at room temperature for 4 hours.

DMF was evaporated under vacuum and the residue was taken up with water (300 ml) and the product was extracted with Et₂O. All the organic layers were combined together, dried over Na₂SO₄ anhydrous, filtered and concentrated under vacuum to give a crude product which was purified by silica gel chromatography (column size 340 g SNAP using Cy:EtOAc=9:1 to Cy:EtOAc=7:3). It was recovered the title compound D131 (5.63 g). UPLC: (Basic Gen_QC): rt=1.31 minutes, peak observed: 418 (M+1). C₂₇H₃₅NOSi requires 417.

### Description 132

### (2-{[(1,1-dimethylethyl)(diphenyl)silyl]oxy}-1-methylpropyl)amine (D132)

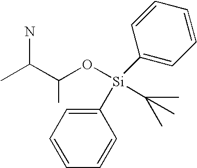

(2-{[(1,1-dimethylethyl)(diphenyl)silyl]oxy}-1-methylpropyl)(phenylmethyl)amine D131 (5.63 g) was dissolved in MeOH (100 ml) then Pd/C (0.143 g, 1.348 mmol) was added and the reaction was hydrogenated in a Buchi reactor under 5 atmospheres pressure of hydrogen at 60° C. for 24 hours. The catalyst was filtered off and the solution was concentrated under vacuum to give a crude which was purified by SCX chromatography (Column size 70 g). It was recovered the title compound D132 (4.3 g). UPLC: (Basic Gen_QC): rt=1.31 minutes, peak observed: 329 (M+2). C₂₀H₂₉NOSi requires 327.

### Description 133

### 2-chloro-N-(2-hydroxy-1-methylpropyl)-6-methyl-3-pyridinecarboxamide (D133)

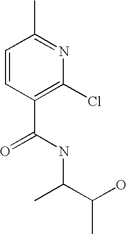

2-chloro-6-methyl-3-pyridinecarboxylic acid (2.05 g, 11.95 mmol) was dissolved in 5 ml of DMF then TBTU (4.22 g, 13.14 mmol) and DIPEA (4.17 ml, 23.90 mmol) were added and the mixture was stirred at room temperature for 1 hour. (2-{[(1,1-dimethylethyl)(diphenyl)silyl]oxy}-1-methylpropyl)amine D132 (4.30 g) dissolved in 5 ml of DMF was added and the reaction was stirred at room temperature for two hours. All volatiles were removed under vacuum (rotary evaporator 55° C.) and the residue was taken up with DCM (10 ml) and it was washed with NaHCO₃ saturated solution (10 ml). The organic phase was dried over Na₂SO₄ anhydrous, filtered and TBAF (11.95 ml, 11.95 mmol) was added. The reaction was stirred for 2 hours at room temperature. All the volatiles were removed under vacuum. The resulting crude product was purified by silica gel chromatography (Biotage SP—Column size 100 g using Cy:EtOAc=8:2 to 2:8).

It was recovered the title compound D133 (1.26 g).

UPLC: (Basic Gen_QC): rt=0.43 minutes, peak observed: 243 (M+1). C₁₁H₁₅ClN₂O₂ requires 242.

### Description 134

### 2-chloro-6-methyl-N-(1-methyl-2-oxopropyl)-3-pyridinecarboxamide (D134)

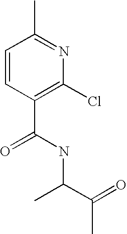

2-chloro-N-(2-hydroxy-1-methylpropyl)-6-methyl-3-pyridinecarboxamide D133 (1.26 g) was dissolved in DCM (100 ml) then DMP (2.202 g, 5.19 mmol) was added and the reaction was stirred for 2 hours at room temperature. 20 ml of aqueous sodium thiosulfate saturated solution and 20 ml of aqueous NaHCO₃ saturated solution were added and the mixture was stirred for 1 hour at room temperature. The organic phase was separated, dried over Na₂SO₄ anhydrous, filtered and concentrated under vacuum to give a crude product which was purified by silica gel chromatography (Biotage SP—column size 100 g, using Cy:EtOAc=8:2 to Cy:EtOAc=5:5). It was recovered the title compound D134 (1.05 g). UPLC: (Basic Gen_QC): rt=0.47 minutes, peak observed: 241 (M+1). C₁₁H₁₃ClN₂O₂ requires 240.

### Description 135

### 2-chloro-3-(4,5-dimethyl-1,3-oxazol-2-yl)-6-methylpyridine (D135)

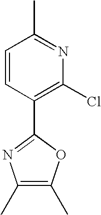

2-chloro-6-methyl-N-(1-methyl-2-oxopropyl)-3-pyridinecarboxamide D134 (1.05 g) was dissolved in THF (35 ml) then Burgess reagent (1.248 g, 5.24 mmol) was added and the mixture was stirred at room temperature for 2 hours. The reaction was not complete and Burgess reagent (1.248 g, 5.24 mmol) was added stirring at room temperature overnight. All volatiles were removed under vacuum and the residue was partitioned between NaHCO₃ (saturated solution 40 ml) and EtOAc. The organic phases were collected together, dried over Na₂SO₄ anhydrous, filtered trough a phase separator tube and concentrated under vacuum to give a crude product which was purified by silica gel chromatography (Biotage SP—column size SNAP 100 g eluting with Cy:EtOAc=8:2 to 2:8). It was recovered the title compound D135 (525 mg). UPLC: (Basic Gen_QC): rt=0.75 minutes, peak observed: 223 (M+1). C₁₁H₁₁ClN₂O requires 222. ¹H NMR (400 MHz, CDCl₃) δ ppm 8.18 (m, 1H) 7.19 (m, 1H) 2.60 (s, 3H) 2.36 (s, 3H) 2.20 (s, 3H).

### Description 136

### 3-(4,5-dimethyl-1,3-oxazol-2-yl)-2-ethenyl-6-methylpyridine (D136)

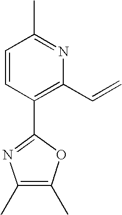

2-chloro-3-(4,5-dimethyl-1,3-oxazol-2-yl)-6-methylpyridine D135 (0.535 g), Pd(Ph₃P)₄ (0.222 g, 0.192 mmol), 2-ethenyl-4,4,5,5-tetramethyl-1,3,2-dioxaborolane (0.448 ml, 2.64 mmol) and potassium carbonate (0.664 g, 4.81 mmol) were mixed together, then water (2 ml) and 1,4-Dioxane (6 ml) were added. The mixture was stirred at 80° C. for 2 hours and 30 minutes: not complete conversion was observed. The solvents were evaporated at reduced pressure and the residue partitioned between NaHCO₃ (saturated solution) (20 ml) and EtOAc (10 ml); water layer extracted with EtOAc. The organic phases were joined and dried over Na₂SO⁴ and evaporated at reduced pressure, obtaining a crude product containing starting material and desired product, so 2-ethenyl-4,4,5,5-tetramethyl-1,3,2-dioxaborolane (0.448 ml, 2.64 mmol), Pd(Ph₃P)₄ (0.222 g, 0.192 mmol) and potassium carbonate (0.664 g, 4.81 mmol) followed by 1,4-Dioxane (6 ml) and water (2 ml) were added and the reaction was stirred at 95° C. for 2 hours: complete conversion was observed. The solvents were evaporated at reduced pressure and the residue partitioned between NaHCO₃ (saturated solution) and EtOAc; water layer extracted with EtOAc. The organic phases were joined and dried over Na₂SO⁴ and evaporated at reduced pressure, obtaining the target material which was purified by silica gel chromatography (Biotage SP—column size SNAP 50 g using Cy:EtOAc=8:2 to Cy:EtOAc=4:60). It was recovered the title compound D136 (275 mg). UPLC: (Basic Gen_QC): rt=0.86 minutes, peak observed: 215 (M+1). C₁₃H₁₄N₂O requires 214. ¹H NMR (400 MHz, CDCl₃) δ ppm 8.08 (d, 1H) 7.97-7.75 (m, 1H) 7.12 (d, 1H) 6.56 (m, 1H) 5.59 (m, 1H) 2.62 (s, 3H) 2.34 (s, 3H) 2.19 (s, 3H).

### Description 137

### 3-(4,5-dimethyl-1,3-oxazol-2-yl)-6-methyl-2-pyridinecarbaldehyde (D137)

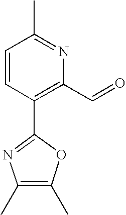

3-(4,5-dimethyl-1,3-oxazol-2-yl)-2-ethenyl-6-methylpyridine D136 (275 mg) was dissolved in THF (10 ml) and water (10 ml). To this stirred mixture a solution of osmium tetroxide (4% in water) (0.101 ml, 0.013 mmol) was added over 30 seconds and the resulting mixture was then stirred at room temperature for 10 minutes (the mixture became very dark) Sodium periodate (1647 mg, 7.70 mmol) was then added in one portion and the resulting mixture (the former dark colour became clear) was left to stir at room temperature for 70 minutes (white precipitate formed). The mixture was then partitioned between NaHCO₃ saturated solution and Et₂O; water layer extracted with Et₂O. The organic phases were joined and dried over Na₂SO₄ and evaporated at reduced pressure, obtaining the title compound D137 as brown solid (280 mg). UPLC: (Basic Gen_QC): rt=0.62 minutes, peak observed: 217 (M+1). C₁₂H₁₂N₂O₂ requires 216. ¹H NMR (400 MHz, CDCl₃) δ ppm 10.77 (s, 1H) 8.23 (d, 1H) 7.43 (d, 1H) 2.73 (s, 3H) 2.37 (s, 3H) 2.20 (s, 3H).

### Description 138

### 3-(4,5-dimethyl-1,3-oxazol-2-yl)-6-methyl-2-pyridinecarboxylic acid (D138)

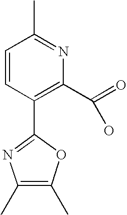

3-(4,5-dimethyl-1,3-oxazol-2-yl)-6-methyl-2-pyridinecarbaldehyde D137 (280 mg) was dissolved in DMSO (5 ml) and pH=3 buffer solution (3 ml) and the mixture was chilled at 0° C. sodium chlorite (3.88 ml, 3.88 mmol) was dropped into the mixture over 10 minutes, then the stirring was continued at room temperature. After 2 hours the reaction was not complete. New pH=3 buffer solution (3 ml), followed by new sodium chlorite (3.88 ml, 3.88 mmol) were dropped into the mixture, which was then stirred at room temperature for other 2 hours. The whole dark mixture has been loaded onto a C18-70 g column [pre-conditioned with 3 CV of methanol and 3 CV of water), firstly eluted with water (7 CV), then with methanol (7 CV)]. It was obtained the title compound D138 (252 mg). UPLC: (Basic GEN_QC): rt=035 minutes, peak observed: 233 (M+1). C₁₂H₁₂N₂O₃ requires 232. ¹H NMR (400 MHz, DMSO-d₆) δ ppm 8.16 (d, 1H) 7.48 (d, 1H) 2.54 (s, 3H) 2.30 (s, 3H) 2.08 (s, 3H).

### EXAMPLES

In the following Examples the relative stereochemistry of the compounds is derived from the stereochemistry of the previous intermediates from which the compounds were synthesised. In some Examples the relative stereochemistry has been confirmed on the final compounds as well. In most Examples the final compounds are present as a mixture of conformers of variable ratio according to the specific Example. For Example E3 is assigned the TRANS configuration based on the stereochemistry of the intermediate D14, the product is present as a mixture of conformers (ratio of approximately 75/25).

### Example 1

### N-[((1R,4S,6R)-3-{[6-methyl-3-(propyloxy)-2-pyridinyl]carbonyl}-3-azabicyclo[4.1.0]hept-4-yl)methyl]-5-(trifluoromethyl)-2-pyridinamine (HCl salt) (E1)

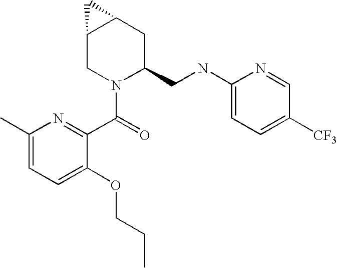

To a solution of 6-methyl-3-(propyloxy)-2-pyridinecarboxylic acid D35 (0.0293 g) in DMF (1 ml), DIPEA (0.14 ml, 0.82 mmol) and TBTU (0.0613 g, 0.19 mmol) were added and the reaction mixture left under stirring at room temperature for 30 minutes. A solution of N-[(1R,4S,6R)-3-azabicyclo[4.1.0]hept-4-ylmethyl]-5-(trifluoromethyl)-2-pyridinamine D14 (0.037 g) in DMF (1 ml) was added. The reaction mixture was stirred for 1 hour, diluted with brine and extracted with DCM. The organic phase was separated, dried (Na₂SO₄), filtered and concentrated under reduced pressure. The residue was purified by flash chromatography (on silica —NH₂ cartridge, Biotage SP 25 M, DCM 100) to afford the free base of the title compound (0.043 g, 0.096 mmol, 70% yield). MS: (ES/+) m/z: 449 (M+1). C₂₃H₂₇F₃N₄O₂ requires 448. The free base (0.043 g, 0.096 mmol) was dissolved in anhydrous DCM (1 ml) and a 1 M HCl solution in Et₂O (0.14 ml, 0.14 mmol) was added and the mixture left under stirring for 1 hour. Volatiles were removed under reduced pressure and the resulting solid triturated with Et₂O to give the title compound E1 (0.046 g) as a yellow solid. UPLC (Basic GEN_QC): rt1=0.77 minutes and rt2=0.78 minutes (rotamers present), peaks observed: 449 (M+1−HCl). C₂₃H₂₈F₃ClN₄O₂ requires 484. ¹H NMR [the TRANS relative stereochemistry is derived from the stereochemistry of the previous intermediate D14. The product is present as a mixture of conformers (ratio ca. 70/30). The assignment is provided for the major component] (500 MHz, DMSO-d₆) δ (ppm): 7.94-8.10 (m, 1H), 7.08-7.89 (m, 4H), 6.63 (d, 1H), 4.49 (d, 1H), 3.28-3.87 (m, 6H), 2.12 (s, 3H), 1.57-1.88 (m, 4H), 0.82-1.15 (m, 5H), 0.65-0.76 (m, 1H), 0.09-0.19 (m, 1H).

### Example 2

### N-({(1R,4S,6R)-3-[(6-methyl-2-pyridinyl)carbonyl]-3-azabicyclo[4.1.0]hept-4-yl}methyl)-5-(trifluoromethyl)-2-pyridinamine (HCl salt) (E2)

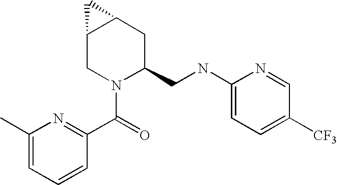

To a solution of 6-methyl-2-pyridinecarboxylic acid (Aldrich #462128) (0.0205 g, 0.15 mmol) in DMF (1 ml), DIPEA (0.026 ml, 0.15 mmol) and TBTU (0.0479 g, 0.15 mmol) were added and the reaction mixture left under stirring at room temperature for 1 hour. A solution of N-[(1R,4S,6R)-3-azabicyclo[4.1.0]hept-4-ylmethyl]-5-(trifluoromethyl)-2-pyridinamine D14 (0.027 g) in DMF (1 ml) was added. The reaction mixture was stirred for 2 hours at room temperature and evaporated to dryness under reduced pressure. The residue was purified by flash chromatography on silica gel (Biotage SP 10 g SNAP, from Cy 100 to Cy/EtOAc 50/50); and then on silica —NH cartridge (Biotage SP4 12M, from Cy 100 to Cy/EtOAc 60/40) to afford the free base of the title compound (0.0123 g, 0.031 mmol, 31% yield). UPLC (Acid FINAL_QC): rt1=0.85 minutes, peak observed: 391 (M+1). C₂₀H₂₁F₃N₄O requires 390. ¹H NMR [the TRANS relative stereochemistry is derived from the stereochemistry of the previous intermediate D14. The product is present as a mixture of conformers (ratio ca. 75/25). The assignment is provided for the major component] (400 MHz, CDCl₃) δ (ppm): 8.32 (bs, 1H), 7.70 (t, 1H), 7.43-7.61 (m, 2H), 7.40 (d, 1H), 7.16-7.26 (m, 1H), 6.52 (d, 1H), 4.73 (d, 1H), 4.01-4.21 (m, 1H), 3.51-3.75 (m, 1H), 3.08-3.33 (m, 2H), 2.59 (s, 3H), 0.94-1.94 (m, 4H), 0.79-0.90 (m, 1H), 0.16 (q, 1H). The free base (0.0123 g, 0.031 mmol) in DCM (1 ml) was cooled down to 0° C. and a 1 M HCl solution in Et₂O (0.05 ml, 0.05 mmol) was added. Volatiles were removed under reduced pressure and the resulting solid triturated with Et₂O to give the title compound E2 (0.0134 g) as a white solid. MS: (ES/+) m/z: 391 (M+1−HCl). C₂₀H₂₂F₃ClN₄O requires 426.

### Example 3

### N-[((1R,4S,6R)-3-{[6-methyl-3-(methyloxy)-2-pyridinyl]carbonyl}-3-azabicyclo[4.1.0]hept-4-yl)methyl]-5-(trifluoromethyl)-2-pyridinamine (HCl salt) (E3)

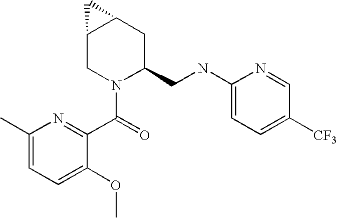

To a solution of 6-methyl-3-(methyloxy)-2-pyridinecarboxylic acid D37 (0.0407 g) in DMF (1 ml), DIPEA (0.053 ml, 0.30 mmol) and TBTU (0.098 g, 0.30 mmol) were added and the reaction mixture left under stirring at room temperature for 1 hour. A solution of N-[(1R,4S,6R)-3-azabicyclo[4.1.0]hept-4-ylmethyl]-5-(trifluoromethyl)-2-pyridinamine D14 (0.055 g) in DMF (1 ml) was added. The reaction mixture was stirred for 2 hours at room temperature and evaporated to dryness under reduced pressure. The residue was purified by flash chromatography (on silica —NH cartridge, Biotage SP SNAP 10 g, from Cy 100 to Cy/EtOAc 50/50) to afford the free base of the title compound (0.045 g, 0.11 mmol, 53% yield). MS: (ES/+) m/z: 421 (M+1). C₂₁H₂₃F₃N₄O₂ requires 420.

¹H NMR [the TRANS relative stereochemistry is derived from the stereochemistry of the previous intermediate D14. The product is present as a mixture of conformers (ratio ca. 75/25). The assignment refers to the major component] (500 MHz, DMSO-d₆) δ (ppm): 7.92-8.03 (m, 1H), 7.37-7.62 (m, 2H), 7.02-7.32 (m, 2H), 6.41-6.55 (m, 1H), 4.45 (d, 1H), 3.60 (s, 3H), 3.29-3.59 (m, 4H), 2.15 (s, 3H), 1.60-1.83 (m, 2H), 1.05-1.13 (m, 1H), 0.92-1.02 (m, 1H), 0.64-0.74 (m, 1H,) 0.06-0.16 (m, 1H).

The free base (0.045 g, 0.11 mmol) was dissolved in DCM (1 ml) and a 1 M HCl solution in Et₂O (0.16 ml, 0.16 mmol) was added. Volatiles were removed under reduced pressure and the resulting solid triturated with Et₂O (3 ml) to give the title compound E3 (0.048 g). UPLC (Acid FINAL_QC): rt1=0.87 minutes and rt2=0.89 minutes (rotamers present), peaks observed: 421 (M+1−HCl). C₂ᵢH₂₄F₃ClN₄O₂ requires 456.

### Example 4

### N-[((1R,4S,6R)-3-{[3-(ethyloxy)-6-methyl-2-pyridinyl]carbonyl}-3-azabicyclo[4.1.0]hept-4-yl)methyl]-5-(trifluoromethyl)-2-pyridinamine (HCl salt) (E4)

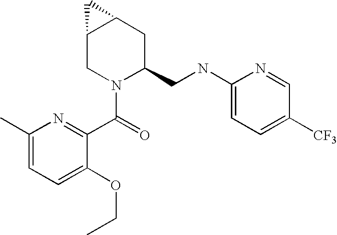

To a solution of 3-(ethyloxy)-6-methyl-2-pyridinecarboxylic acid D39 (0.0441 g) in DMF (1 ml), DIPEA (0.053 ml, 0.30 mmol) and TBTU (0.098 g, 0.30 mmol) were added and the reaction mixture left under stirring at room temperature for 1 hour. A solution of N-[(1R,4S,6R)-3-azabicyclo[4.1.0]hept-4-ylmethyl]-5-(trifluoromethyl)-2-pyridinamine D14 (0.055 g) in DMF (1 ml) was added. The reaction mixture was stirred for 2 hours at room temperature and evaporated to dryness under reduced pressure. The residue was purified by flash chromatography on NH cartridge (Biotage SP SNAP 10 g, from Cy 100 to Cy/EtOAc 50/50) to afford the free base of the title compound (0.045 g). MS: (ES/+) m/z: 435 (M+1). C₂₂H₂₅F₃N₄O₂ requires 434.

The free base (0.045 g) was dissolved in DCM (1 ml) and a 1 M HCl solution in Et₂O (0.14 ml, 0.14 mmol) was added. Volatiles were removed under reduced pressure and the resulting solid triturated with Et₂O (3 ml) to give the title compound E4 (0.042 g). UPLC (Basic GEN_QC): rt1=0.92 min and 0.93 min, peak observed: 435 (M+1−HCl). C₂₂H₂₆ClF₃N₄O₂ requires 470. ¹H NMR [the TRANS relative stereochemistry is derived from the stereochemistry of the previous intermediate D14. The product is present as a mixture of conformers (ratio ca. 70/30). The assignment is provided for the major component] ¹H NMR (500 MHz, DMSO-d₆) δ (ppm): 7.63-8.19 (m, 3H), 7.14-7.58 (m, 2H), 6.64-6.77 (m, 1H), 4.49 (d, 1H), 3.89-4.21 (m, 2H), 3.29-3.78 (m, 4H), 2.14 (s, 3H), 1.69-1.91 (m, 2H), 1.20-1.39 (m, 3H), 0.92-1.18 (m, 2H), 0.65-0.76 (m, 1H), 0.13-0.20 (m, 1H).

The following compounds were prepared using a similar procedure to that described for Example 4 (in some examples the solvent used was DCM instead of DMF and/or the order of addition of the reagents was different). Each compound was obtained by amide coupling of N-[(1R,4S,6R)-3-azabicyclo[4.1.0]hept-4-ylmethyl]-heteroarylamine derivative with the appropriate carboxylic acid or suitable salt thereof. This is provided merely for assistance to the skilled chemist. The starting material may not necessarily have been prepared from the batch referred to.

Unless specified the free base was not treated with the HCl solution to give the corresponding HCl salt.

| | | Amide coupling | |
|---|---|---|---|
| No. | | Reactants | Characterising data |
| E5 | 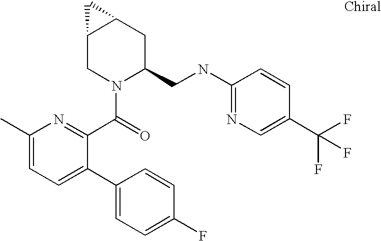 | D14 and D52 | N-[((1R,4S,6R)-3-{[3-(4-fluorophenyl)-6-methyl-2- pyridinyl]carbonyl}-3-azabicyclo[4.1.0]hept-4- yl)methyl]-5-(trifluoromethyl)-2-pyridinamine ¹H NMR (400 MHz, CDCl₃) δ ppm 0.88-0.55 (m, 1 H) 0.36-0.58 (m, 1 H) 0.70-1.06 (m, 2 H) 1.38-1.74 (m, 2 H) 2.23 (s, 3 H) 3.20-3.48 (m, 4 H) 4.33 (d, 1 H) 6.50 (d, 1 H) 7.14-7.51 (m, 6 H) 7.53-7.84 (m, 2 H) 7.99-8.11 (m, 1 H) UPLC (Basic GEN_QC): rt1 = 0.96 min, peak observed: 485 (M + 1). C₂₆H₂₄F₄N₄O requires 484. |
| E6 | 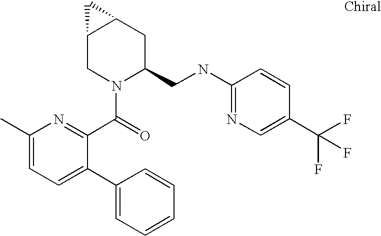 | D14 and D66 | N-({(1R,4S,6R)-3-[(6-methyl-3-phenyl-2- pyridinyl)carbonyl]-3-azabicyclo[4.1.0]hept-4- yl}methyl)-5-(trifluoromethyl)-2-pyridinamine ¹H NMR (400 MHz, CDCl₃) δ ppm 0.86-0.60 (m, 1 H) 0.32-0.52 (m, 1 H) 0.62-1.12 (m, 2 H) 1.31-1.75 (m, 2 H) 2.21 (s, 3 H) 3.16-3.57 (m, 4 H) 4.33 (d, 1 H) 6.44- 6.67 (m, 1 H) 7.15-7.54 (m, 7 H) 7.51-7.82 (m, 2 H) 8.02-8.10 (m, 1 H) UPLC (Basic GEN_QC): rt1 = 0.97 min, peak observed: 467 (M + 1). C₂₆H₂₅F₃N₄O requires 466. |
| E7 | 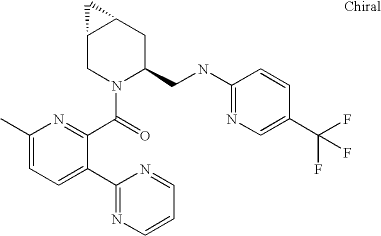 | D14 and D46 | N-[((1R,4S,6R)-3-{[6-methyl-3-(2-pyrimidinyl)-2- pyridinyl]carbonyl}-3-azabicyclo[4.1.0]hept-4- yl)methyl]-5-(trifluoromethyl)-2-pyridinamine ¹H NMR (500 MHz, CDCl₃) δ ppm 0.51-0.60 (m, 1 H) 0.79-0.92 (m, 1 H) 1.01-1.20 (m, 2 H) 1.73-1.87 (m, 1 H) 2.07-2.28 (m, 1 H) 2.64 (s, 3 H) 3.11-3.19 (m, 1 H) 3.20-3.29 (m, 1 H) 3.88-3.97 (m, 1 H) 3.97-4.16 (m, 1 H) 4.78 (d, 1 H) 6.53-6.60 (m, 1 H) 7.27-7.36 (m, 2 H) 7.43 (br. s., 1 H) 7.50-7.61 (m, 1 H) 8.31-8.42 (m, 1 H) 8.51-8.57 (m, 1 H) 8.81-8.91 (m, 2 H) UPLC (Basic GEN_QC): rt1 = 0.90 min, peak observed 469 (M + 1). C₂₄H₂₃F₃N₆O requires 468 A large scale synthesis for E7 is given in example 48 |
| E8 | 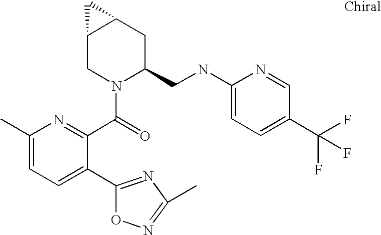 | D14 and D64 | N-[((1R,4S,6R)-3-{[6-methyl-3-(3-methyl-1,2,4- oxadiazol-5-yl)-2-pyridinyl]carbonyl}-3- azabicyclo[4.1.0]hept-4-yl)methyl]-5-(trifluoromethyl)- 2-pyridinamine ¹H NMR (500 MHz, CDCl₃) δ ppm 0.58-0.91 (m, 2 H) 1.01-1.12 (m, 1 H) 1.09-1.25 (m, 1 H) 1.73-1.89 (m, 1 H) 1.93-2.05 (m, 1 H) 2.50 (s, 3 H) 2.66 (s, 3 H) 3.12- 3.22 (m, 1 H) 3.28 (d, 1 H) 3.67-3.78 (m, 1 H) 3.98- 4.13 (m, 1 H) 4.81 (d, 1 H) 6.54 (d, 1 H) 6.77 (br. s., 1 H) 7.36 (d, 1 H) 7.50-7.61 (m, 1 H) 8.23-8.39 (m, 2 H). UPLC (Basic GEN_QC): rt1 = 0.90 min, peak observed 473 (M + 1). C₂₃H₂₃F₃N₆O₂ requires 472 |
| E9 | 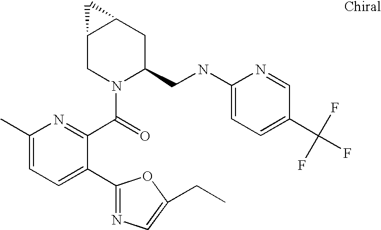 | D14 and D58 | N-[((1R,4S,6R)-3-{[3-(5-ethyl-1,3-oxazol-2-yl)-6-methyl- 2-pyridinyl]carbonyl}-3-azabicyclo[4.1.0]hept-4- yl)methyl]-5-(trifluoromethyl)-2-pyridinamine ¹H NMR (500 MHz, DMSO-d₆) δ ppm 0.14-0.34 (m, 1 H) 0.67-0.79 (m, 1 H) 0.89-1.16 (m, 2 H) 1.17-1.28 (m, 3 H) 1.42-1.99 (m, 2 H) 2.38 (s, 3 H) 2.66-2.80 (m, 2 H) 3.34-3.67 (m, 4 H) 4.42 (d, 1 H) 6.30-6.53 (m, 1 H) 7.01 (s, 1 H) 7.32-7.47 (m, 2 H) 7.48-7.57 (m, 1 H) 7.90-8.06 (m, 1 H) 8.15 (d, 1 H) UPLC (Basic GEN_QC): rt1 = 0.97 min, rt2 = 1.00 min (rotamers peak) peaks observed 486 (M + 1). C₂₅H₂₆F₃N₅O requires 485 |
| E10 | 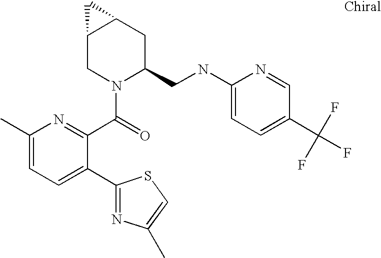 | D14 and D48 | N-[((1R,4S,6R)-3-{[6-methyl-3-(4-methyl-1,3-thiazol-2- yl)-2-pyridinyl]carbonyl}-3-azabicyclo[4.1.0]hept-4- yl)methyl]-5-(trifluoromethyl)-2-pyridinamine ¹H NMR (400 MHz, DMSO-d₆) δ ppm 0.04-0.69 (m, 2 H) 0.76-1.43 (m, 2 H) 1.63-1.73 (m, 2 H) 2.27 (s, 3 H) 2.31 (s, 3 H) 3.27-3.62 (m, 4 H) 4.23 (d, 1 H) 6.20-6.41 (m, 1 H) 7.22-7.35 (m, 3 H) 7.44 (d, 1 H) 7.93 (br. s., 1 H) 8.00 (d, 1 H) UPLC (Basic GEN_QC): rt1 = 0.95 min, peak observed 488 (M + 1). C₂₄H₂₄F₃N₅OS requires 487 |
| E11 | 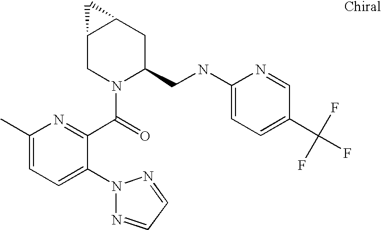 | D14 and D50 | N-[((1R,4S,6R)-3-{[6-methyl-3-(2H-1,2,3-triazol-2-yl)-2- pyridinyl]carbonyl}-3-azabicyclo[4.1.0]hept-4- yl)methyl]-5-(trifluoromethyl)-2-pyridinamine ¹H NMR (500 MHz, DMSO-d₆) δ ppm 0.12-0.23 (m, 1 H) 0.65-0.74 (m, 1 H) 0.90-1.02 (m, 1 H) 1.01-1.13 (m, 1 H) 1.58-1.78 (m, 2 H) 2.35 (s, 3 H) 3.33-3.41 (m, 2 H) 3.48-3.76 (m, 2 H) 4.36 (d, 1 H) 6.42-6.58 (m, 1 H) 7.29- 7.47 (m, 2 H) 7.54-7.61 (m, 1 H) 8.00-8.09 (m, 1 H) 8.10-8.19 (m, 3 H)] UPLC (Acid QC_POS _50-800): rt1 = 0.90 min, peak observed 458 (M + 1). C₂₂H₂₂F₃N₇O requires 457 |
| E12 | 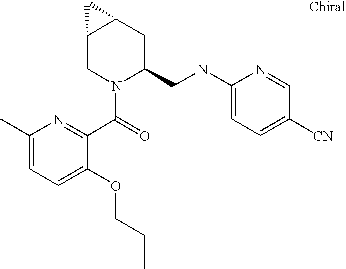 | D18 and D35 | 6-{[((1R,4S,6R)-3-{[6-methyl-3-(propyloxy)-2- pyridinyl]carbonyl}-3-azabicyclo[4.1.0]hept-4- yl)methyl]amino}-3-pyridinecarbonitrile (free base) UPLC (Acid FINAL_QC): rt = 0.68 and 0.69 min, peak observed: 406 (M + 1). C₂₃H₂₇N₅O₂ requires 405. ¹H NMR (500 MHz, DMSO-d₆) δ (ppm): 7.94 (bs, 1 H), 7.54-7.61 (m, 2 H), 7.18 (d, 1 H), 6.96 (d, 1 H), 6.36 (bs, 1 H), 4.32-4.38 (m, 1 H), 3.58-3.70 (m, 1 H), 3.07- 3.41 (m, 5 H), 2.01 (bs, 3 H), 1.57-1.66 (m, 1 H), 1.43- 1.58 (m, 2 H), 0.90-1.03 (m, 1 H), 0.73-0.87 (m, 5 H), 0.51-0.63 (m, 1 H), 0.03-0.08 (m, 1 H) 6-{[((1R,4S,6R)-3-{[6-methyl-3-(propyloxy)-2- pyridinyl]carbonyl}-3-azabicyclo[4.1.0]hept-4- yl)methyl]amino}-3-pyridinecarbonitrile Hydrochloride UPLC (Acid FINAL_QC): rt = 0.68 and 0.69 min, peak observed: 406 (M + 1 − HCl). C₂₃H₂₈ClN₅O₂ requires 441. |
| E13 | 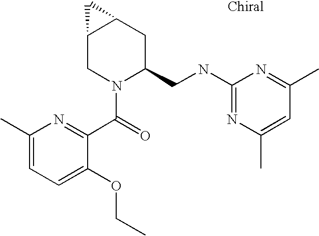 | D20 and D39 | N-[((1R,4S,6R)-3-{[3-(ethyloxy)-6-methyl-2- pyridinyl]carbonyl}-3-azabicyclo[4.1.0]hept-4- yl)methyl]-4,6-dimethyl-2-pyrimidinamine (free base) MS: (LS/+) m/z: 396 (M + 1). C₂₂H₂₉N₅O₂ requires 395. ¹H NMR ¹H NMR (500 MHz, DMSO-d₆) δ ppm: 6.94-7.20 (m, 2 H), 6.28 (s, 1 H), 5.74-6.04 (m, 1 H), 4.75 (d, 1 H), 3.99-4.27 (m, 2 H), 3.22-3.88 (m, 4 H), 2.47 (s, 3 H), 2.25 (s, 6 H), 1.76-2.10 (m, 2 H), 1.45 (t, 3 H), 0.85-1.17 (m, 2 H), 0.72-0.81 (m, 1 H), 0.30-0.37 (m, 1 H). N-[((1R,4S,6R)-3-{[3-(ethyloxy)-6-methyl-2- pyridinyl]carbonyl}-3-azabicyclo[4.1.0]hept-4- yl)methyl]-4,6-dimethyl-2-pyrimidinamine Hydrochloride UPLC (Basic GEN_QC): rt = 0.76 and 0.80 min, peak observed: 396 (M + 1 − HCl). C₂₂H₃₀ClN₅O₂ requires 431. |
| E14 | 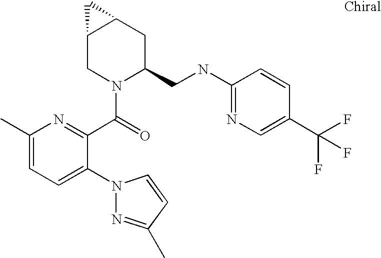 | D14 and D71 | N-[((1R,4S,6R)-3-{[6-methyl-3-(3-methyl-1H-pyrazol-1- yl)-2-pyridinyl]carbonyl}-3-azabicyclo[4.1.0]hept-4- yl)methyl]-5-(trifluoromethyl)-2-pyridinamine ¹H NMR (400 MHz, DMSO-d₆) δ ppm −0.08-0.06 (m, 1 H) 0.65-0.75 (m, 1 H) 0.86-1.16 (m, 3 H) 1.66-1.81 (m, 1 H) 2.28 (s, 3 H) 2.32 (br. s., 3 H) 3.29-3.43 (m, 2 H) 3.47-3.77 (m, 2 H) 4.38 (d, 1 H) 6.33-6.37 (m, 1 H) 6.45- 6.60 (m, 1 H) 7.35 (d, 1 H) 7.43-7.50 (m, 1 H) 7.61 (d, 1 H) 7.83-8.06 (m, 2 H) 8.04-8.14 (m, 1 H) UPLC (Basic QC_POS_50-800): rt1 = 0.90 minutes and rt2 = 0.92 minutes (rotamers present), peaks observed: 471 (M + 1). C₂₄H₂₅F₃N₆O requires 470. |
| E15 | 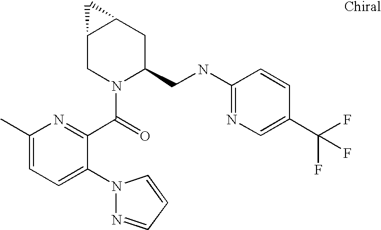 | D14 and D73 | N-[((1R,4S,6R)-3-{[6-methyl-3-(1H-pyrazol-1-yl)-2- pyridinyl]carbonyl}-3-azabicyclo[4.1.0]hept-4- yl)methyl]-5-(trifluoromethyl)-2-pyridinamine ¹H NMR (500 MHz, DMSO-d₆) δ ppm −0.19-0.04 (m, 1 H) 0.58-0.69 (m, 1 H) 0.82-1.12 (m, 3 H) 1.55-1.75 (m, 1 H) 2.27 (br. s., 3 H) 3.24-3.40 (m, 2 H) 3.42-3.65 (m, 2 H) 4.36 (d, 1 H) 6.44-6.59 (m, 2 H) 7.29-7.44 (m, 2 H) 7.59 (d, 1 H) 7.73-7.79 (m, 1 H) 7.96-8.16 (m, 3 H) UPLC (Basic QC_POS_50-800): rt1 = 0.88 minutes and rt2 = 0.89 minutes (rotamers present), peaks observed: 457 (M + 1). C₂₃H₂₃F₃N₆O requires 456. |
| E16 | 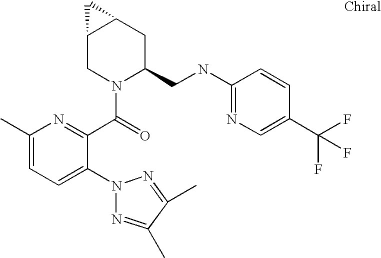 | D14 and D75 | N-[((1R,4S,6R)-3-{[3-(4,5-dimethyl-2H-1,2,3-triazol-2- yl)-6-methyl-2-pyridinyl]carbonyl}-3- azabicyclo[4.1.0]hept-4-yl)methyl]-5-(trifluoromethyl)- 2-pyridinamine ¹H NMR (500 MHz, DMSO-d₆) δ ppm 0.23 (br. s., 1 H) 0.68-0.76 (m, 1 H) 0.79-1.19 (m, 3 H) 1.68-1.78 (m, 1 H) 2.22-2.30 (m, 6 H) 2.50 (s, 3 H) 3.29-3.50 (m, 1 H) 3.51-3.66 (m, 3 H) 4.36 (d, 1 H) 6.51 (br. s., 1 H) 7.41- 7.50 (m, 1 H) 7.52-7.65 (m, 2 H) 8.02-8.17 (m, 2 H) UPLC (Basic QC_POS_50-800): rt1 = 0.98 minutes, peak observed: 486 (M + 1). C₂₄H₂₆F₃N₇O requires 485. |
| E17 | 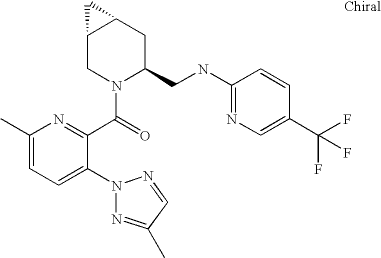 | D14 and D79 | N-[((1R,4S,6R)-3-{[6-methyl-3-(4-methyl-2H-1,2,3- triazol-2-yl)-2-pyridinyl]carbonyl}-3- azabicyclo[4.1.0]hept-4-yl)methyl]-5-(trifluoromethyl)- 2-pyridinamine ¹H NMR (500 MHz, DMSO-d₆) δ ppm 0.17-0.24 (m, 1 H) 0.66-0.76 (m, 1 H) 0.92-1.13 (m, 2 H) 1.59-1.67 (m, 1 H) 1.68-1.78 (m, 1 H) 2.30-2.38 (m, 6 H) 3.27-3.76 (m, 4 H) 4.37 (d, 1 H) 6.50 (br. s., 1 H) 7.35-7.44 (m, 2 H) 7.58 (d, 1 H) 7.85-7.92 (m, 1 H) 8.04 (br. s., 1 H) 8.10 (d, 1H) UPLC (Acid QC_POS_50-800): rt1 = 0.76 minutes and rt2 = 0.80 minutes (rotamers present), peaks observed: 472 (M + 1). C₂₃H₂₄F₃N₇O requires 471. |
| E18 | 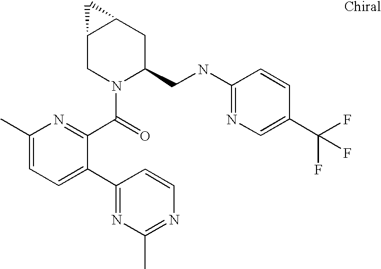 | D14 and D81 | N-[((1R,4S,6R)-3-{[6-methyl-3-(2-methyl-4- pyrimidinyl)-2-pyridinyl]carbonyl}-3- azabicyclo[4.1.0]hept-4-yl)methyl]-5-(trifluoromethyl)- 2-pyridinamine ¹H NMR (500 MHz, DMSO-d₆) δ ppm −0.20-−0.04 (m, 1 H) 0.67-0.75 (m, 1 H) 0.92-1.13 (m, 2 H) 1.31-1.50 (m, 1 H) 1.75-1.85 (m, 1 H) 2.40 (s, 3 H) 2.63 (s, 3 H) 3.36- 3.74 (m, 4 H) 4.28 (d, 1 H) 6.41 (d, 1 H) 7.28-7.70 (m, 4 H) 7.92-8.05 (m, 1 H) 8.11 (d, 1 H) 8.69-8.76 (m, 1 H) UPLC (Acid QC_POS_50-800): rt1 = 0.68 minutes and rt2 = 0.71 minutes (rotamers present), peaks observed: 483 (M + 1). C₂₅H₂₅F₃N₆O requires 482. |
| E19 | 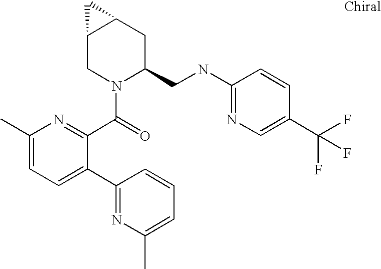 | D14 and D83 | N-({(1R,4S,6R)-3-[(6,6′-dimethyl-2,3′-bipyridin-2′- yl)carbonyl]-3-azabicyclo[4.1.0]hept-4-yl}methyl)-5- (trifluoromethyl)-2-pyridinamine ¹H NMR (500 MHz, DMSO-d₆) δ ppm −0.30 (br. s., 1 H) 0.58-0.67 (m, 1 H) 0.83-1.10 (m, 3 H) 1.61-1.79 (m, 1 H) 2.32 (s, 3 H) 2.50 (s, 3 H) 3.31-3.35 (m, 1 H) 3.44- 3.58 (m, 3 H) 4.27 (d, 1 H) 6.43 (br. s., 1 H) 7.18-7.34 (m, 2 H) 7.37-7.49 (m, 2 H) 7.50-7.58 (m, 1 H) 7.69-7.78 (m, 1H) 7.96 (d, 1H) 8.00-8.08 (m, 1H) UPLC (Acid GEN_QC): rt1 = 0.73 minutes and rt2 = 0.75 minutes (rotamers present), peaks observed: 482 (M + 1). C₂₆H₂₆F₃N₅O requires 481. |
| E20 | 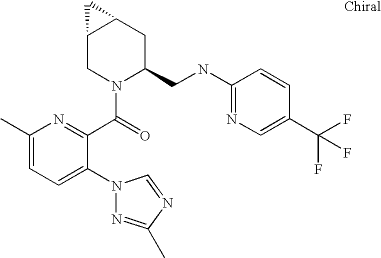 | D14 and D85 | N-[((1R,4S,6R)-3-{[6-methyl-3-(3-methyl-1H-1,2,4- triazol-1-yl)-2-pyridinyl]carbonyl}-3- azabicyclo[4.1.0]hept-4-yl)methyl]-5-(trifluoromethyl)- 2-pyridinamine ¹H NMR (500 MHz, DMSO-d₆) δ ppm −0.20-−0.03 (m, 1 H) 0.63-0.72 (m, 1 H) 0.91-0.99 (m, 1 H) 1.04-1.12 (m, 1 H) 1.39-1.53 (m, 1 H) 1.70-1.81 (m, 1 H) 2.33 (s, 3 H) 2.50-2.52 (m, 9 H) 3.33-3.69 (m, 4 H) 4.34 (d, 1 H) 6.49 (d, 1 H) 7.36-7.53 (m, 2 H) 7.55-7.61 (m, 1 H) 7.92 (d, 1 H) 8.04-8.09 (m, 1 H) 8.64 (s, 1 H) UPLC (Acid GEN_QC): rt1 = 0.68 minutes and rt2 = 0.72 minutes (rotamers present), peaks observed: 472 (M + 1). C₂₃H₂₄F₃N₇O requires 471. ¹H NMR (500 MHz, DMSO-d₆) δ ppm −0.20-−0.03 (m, 1 H), 0.63-0.72 (m, 1 H), 0.91-0.99 (m, 1 H), 1.04-1.12 (m, 1 H), 1.39-1.53 (m, 1 H), 1.70-1.81 (m, 1 H), 2.33 (s, 3 H), 2.50-2.52 (m, 3 H), 3.33-3.69 (m, 4 H), 4.34 (d, 1 H), 6.49 (d, 1 H), 7.36-7.53 (m, 2 H), 7.55-7.61 (m, 1 H), 7.92 (d, 1 H), 8.04-8.09 (m, 1 H), 8.64 (s, 1 H). UPLC (Acid GEN_QC): rt1 = 0.68 minutes and rt2 = 0.72 minutes (rotamers present), peaks observed: 472 (M + 1). C₂₃H₂₄F₃N₇O requires 471. |
| E21 | 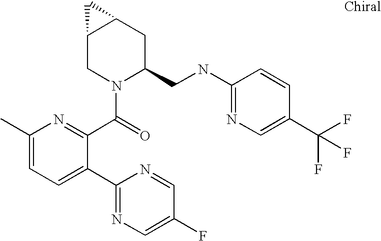 | D14 and D87 | N-[((1R,4S,6R)-3-{[3-(5-fluoro-2-pyrimidinyl)-6-methyl- 2-pyridinyl]carbonyl}-3-azabicyclo[4.1.0]hept-4- yl)methyl]-5-(trifluoromethyl)-2-pyridinamine UPLC (Basic GEN_QC): rt = 0.94 minutes, peak observed: 487 (M + 1). C₂₄H₂₂F₄N₆O requires 486. ¹H NMR (400 MHz, DMSO-d₆) δ ppm 0.20-0.32 (m, 1 H) 0.72-0.83 (m, 1 H) 0.86-1.20 (m, 2 H) 1.54-1.85 (m, 2 H) 2.45 (s, 3 H) 3.20-3.43 (m, 2 H) 3.52-3.76 (m, 2 H) 4.35 (d, 1 H) 6.45 (d, 1 H) 7.30-7.50 (m, 2 H) 7.54 (d, 1 H) 8.05 (br. s., 1 H) 8.24-8.51 (m, 1 H) 8.81-9.10 (m, 2 H) |
| E22 | 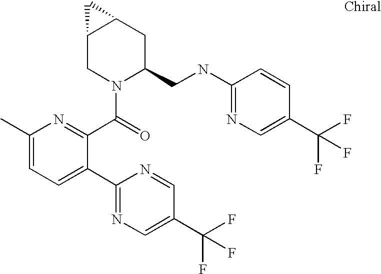 | D14 and D89 | N-{[(1R,4S,6R)-3-({6-methyl-3-[5-(trifluoromethyl)-2- pyrimidinyl]-2-pyridinyl}carbonyl)-3- azabicyclo[4.1.0]hept-4-yl]methyl}-5-(trifluoromethyl)- 2-pyridinamine UPLC (Basic GEN_QC): rt = 1.02 minutes, peak observed: 537 (M + 1). C₂₅H₂₂F₆N₆O requires 536. ¹H NMR (400 MHz, DMSO-d₆) δ ppm 0.21-0.32 (m, 1 H) 0.73-0.85 (m, 1 H) 0.89-1.26 (m, 2 H) 1.53-1.76 (m, 2 H) 2.57 (s, 3 H) 3.24-3.46 (m, 2 H) 3.53-3.83 (m, 2 H) 4.35 (d, 1 H) 6.41 (d, 1 H) 7.35-7.55 (m, 3 H) 7.99 (br. s., 1 H) 8.50 (d, 1 H) 9.25-9.40 (m, 2 H) |
| E23 | 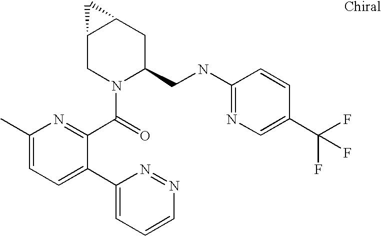 | D14 and D91 | N-[((1R,4S,6R)-3-{[6-methyl-3-(3-pyridazinyl)-2- pyridinyl]carbonyl}-3-azabicyclo[4.1.0]hept-4- yl)methyl]-5-(trifluoromethyl)-2-pyridinamine UPLC (Basic GEN_QC): rt1 = 0.81 minutes and rt2 = 0.83 minutes (rotamers present), peak observed: (M + 1). C₂₄H₂₃F₃N₆O requires 468. ¹H NMR (500 MHz, DMSO-d₆) δ ppm 0.04-0.14 (m, 1 H) 0.61-0.69 (m, 1 H) 0.87-1.11 (m, 3 H) 1.64-1.78 (m, 1 H) 2.44 (s, 3 H) 3.21 (dd, 1 H) 3.28-3.77 (m, 2 H) 3.63- 3.72 (m, 1 H) 4.34 (d, 1 H) 6.71-6.82 (m, 1 H) 7.42-7.51 (m, 2 H) 7.60 (d, 1 H) 7.80-7.87 (m, 1 H) 7.95-8.02 (m, 1 H) 8.10 (d, 1 H) 8.15-8.18 (m, 1 H) 9.27 (d, 1 H) |
| E24 | 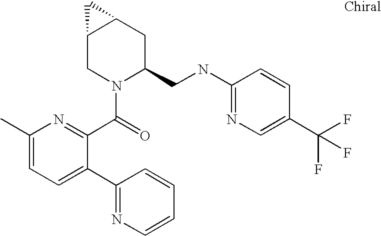 | D14 and D93 | N-({(1R,4S,6R)-3-[(6′-methyl-2,3′-bipyridin-2′- yl)carbonyl]-3-azabicyclo[4.1.0]hept-4-yl}methyl)-5- (trifluoromethyl)-2-pyridinamine UPLC (Basic GEN_QC): rt = 0.90, peak observed: 468 (M + 1). C₂₅H₂₄F₃N₅O requires 467. ¹H NMR (500 MHz, DMSO-d₆) δ ppm −0.18-−0.03 (m, 1 H) 0.59-0.68 (m, 1 H) 0.80-1.10 (m, 3 H) 1.56-1.75 (m, 1H) 2.35 (s, 3 H) 3.30 (dd, 1 H) 3.47-3.70 (m, 3 H) 4.35 (d, 1 H) 6.47-6.56 (m, 1 H) 7.32 (d, 1 H) 7.41 (d, 1 H) 7.43-7.54 (m, 1 H) 7.59 (d, 1 H) 7.65 (d, 1 H) 7.81-7.92 (m, 1 H) 8.01 (d, 1 H) 8.05-8.14 (m, 1 H) 8.59-8.70 (m, 1 H) |
| E25 | 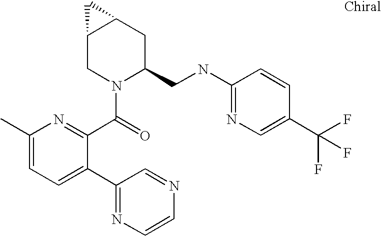 | D14 and D95 | N-[((1R,4S,6R)-3-{[6-methyl-3-(2-pyrazinyl)-2- pyridinyl]carbonyl}-3-azabicyclo[4.1.0]hept-4- yl)methyl]-5-(trifluoromethyl)-2-pyridinamine UPLC (Basic GEN_QC): rt = 0.86, peak observed: 469 (M + 1). C₂₄H₂₃F₃N₆O requires 468. ¹H NMR (500 MHz, DMSO-d₆) δ ppm −0.18-−0.03 (m, 1 H) 0.62-0.72 (m, 1 H) 0.86-1.14 (m, 3 H) 1.66-1.79 (m, 1 H) 2.38 (s, 3 H) 3.25-3.39 (m, 1 H) 3.28-3.65 (m, 2 H) 3.60-3.75 (m, 1 H) 4.35 (d, 1 H) 6.48 (d, 1 H) 7.39 (d, 1 H) 7.44-7.50 (m, 1 H) 7.56 (d, 1 H) 8.02-8.15 (m, 2 H) 8.60- 8.75 (m, 2 H) 8.86-8.94 (m, 1 H) |
| E26 | 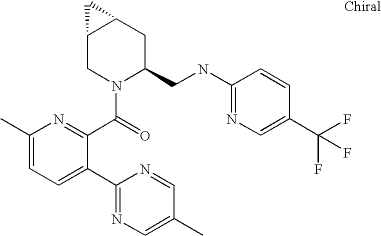 | D14 and D97 | N-[((1R,4S,6R)-3-{[6-methyl-3-(5-methyl-2- pyrimidinyl)-2-pyridinyl]carbonyl}-3- azabicyclo[4.1.0]hept-4-yl)methyl]-5-(trifluoromethyl)- 2-pyridinamine UPLC (Acid GEN_QC): rt1 = 0.74 minutes and rt2 = 0.80 min (rotamers present), peaks observed: 483 (M + 1). C₂₅H₂₅F₃N₆O requires 482. ¹H NMR (400 MHz, DMSO-d₆) δ ppm 0.28-0.35 (m, 1 H) 0.73-0.81 (m, 1 H) 0.81-1.16 (m, 2 H) 1.61-1.84 (m, 2 H) 2.33 (s, 3 H) 2.44 (s, 3 H) 3.35-3.80 (m, 4 H) 4.37 (d, 1 H) 6.42-6.56 (m, 1 H) 7.34-7.47 (m, 2 H) 7.54-7.62 (m, 1 H) 8.05-8.18 (m, 1 H) 8.38 (d, 1 H) 8.73 (s, 2 H). |
| E27 | 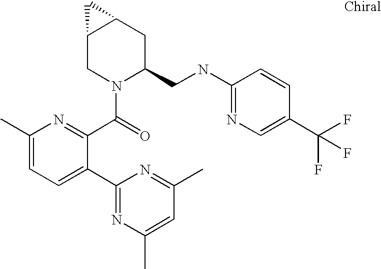 | D14 and D99 | N-[((1R,4S,6R)-3-{[3-(4,6-dimethyl-2-pyrimidinyl)-6- methyl-2-pyridinyl]carbonyl}-3-azabicyclo[4.1.0]hept-4- yl)methyl]-5-(trifluoromethyl)-2-pyridinamine UPLC (Acid GEN_QC): rt1 = 0.76 minutes and rt2 = 0.79 min (rotamers present), peaks observed: 497 (M + 1). C₂₆H₂₇F₃N₆O requires 496. ¹H NMR (500 MHz, DMSO-d₆) δ ppm 0.06-0.16 (m, 1 H) 0.75-0.82 (m, 1 H) 0.97-1.17 (m, 2 H) 1.43-1.63 (m, 1 H) 1.80-1.91 (m, 1 H) 2.42-2.47 (m, 9 H) 3.40-3.68 (m, 4 H) 4.10-4.30 (m, 1 H) 6.25-6.40 (m, 1 H) 7.19 (s, 1 H) 7.32-7.54 (m, 3 H) 7.91-8.02 (m, 1 H) 8.39 (d, 1 H) |
| E28 | 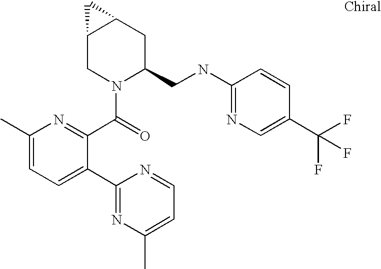 | D14 and D100 | N-[((1R,4S,6R)-3-{[6-methyl-3-(4-methyl-2- pyrimidinyl)-2-pyridinyl]carbonyl}-3- azabicyclo[4.1.0]hept-4-yl)methyl]-5-(trifluoromethyl)- 2-pyridinamine UPLC (Basic GEN_QC): rt = 0.93 minutes, peak observed: 483 (M + 1). C₂₅H₂₅F₃N₆O requires 482. ¹H NMR (500 MHz, DMSO-d₆) δ ppm 0.17-0.24 (m, 1 H) 0.72-0.80 (m, 1 H) 0.92-1.19 (m, 2 H) 1.55-1.67 (m, 1 H) 1.75-1.85 (m, 1 H) 2.44-2.47 (s, 3 H) 2.53-2.55 (s, 3 H) 3.27-3.45 (m, 2 H) 3.56-3.78 (m, 2 H) 4.24-4.35 (d, 1 H) 6.36-6.48 (m, 1 H) 7.32-7.35 (m, 1 H) 7.39-7.44 (m, 2 H) 7.50-7.57 (d, 1 H) 7.98-8.09 (m, 1 H) 8.38-8.44 (d, 1 H) 8.66-8.79 (m, 1 H). |
| E29 | 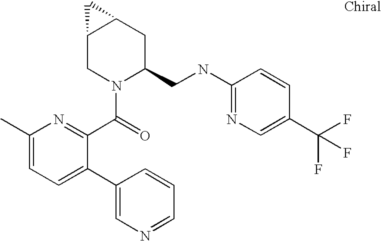 | D14 and D101 | N-({(1R,4S,6R)-3-[(6-methyl-3,3′-bipyridin-2- yl)carbonyl]-3-azabicyclo[4.1.0]hept-4-yl}methyl)-5- (trifluoromethyl)-2-pyridinamine UPLC (Basic GEN_QC): rt1 = 0.80 minutes and rt2 = 0.82 min (rotamers present), peaks observed: 468 (M + 1). C₂₅H₂₄F₃N₅O requires 467. ¹H NMR (500 MHz, DMSO-d₆) δ ppm −0.73 (br. s., 1 H) 0.40-0.53 (m, 1 H) 0.74-1.03 (m, 3 H) 1.56-1.66 (m, 1 H) 2.25 (s, 3 H) 3.23-3.44 (m, 4 H) 4.34 (d, 1 H) 6.52 (d, 1 H) 7.30 (d, 1 H) 7.41-7.50 (m, 2 H) 7.59 (dd, 1 H) 7.75-7.89 (m, 2 H) 8.05 (s, 1 H) 8.54-8.65 (m, 2 H) |
| E30 | 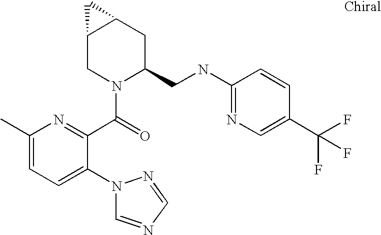 | D14 and D103 | N-[((1R,4S,6R)-3-{[6-methyl-3-(1H-1,2,4-triazol-1-yl)-2- pyridinyl]carbonyl}-3-azabicyclo[4.1.0]hept-4- yl)methyl]-5-(trifluoromethyl)-2-pyridinamine UPLC (Basic GEN_QC): rt = 0.80 minutes, peak observed: 458 (M + 1). C₂₂H₂₂F₃N₇O requires 457. ¹H NMR (500 MHz, DMSO-d₆) δ ppm −0.22-−0.09 (m, 1 H) 0.61-0.70 (m, 1 H) 0.87-1.13 (m, 2 H) 1.31-1.49 (m, 1 H) 1.69-1.80 (m, 1 H) 2.32 (s, 3 H) 3.28-3.65 (m, 4 H) 4.33 (d, 1 H) 6.50 (d, 1 H) 7.40-7.47 (m, 2 H) 7.57 (d, 1 H) 7.97 (d, 1 H) 8.04-8.09 (m, 1 H) 8.25 (s, 1 H) 8.81 (s, 1 H) |
| E31 | 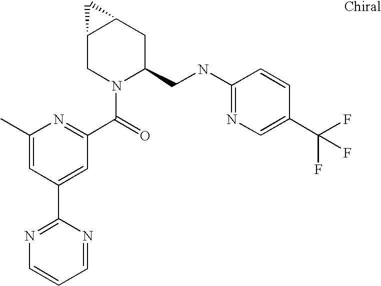 | D14 and D105 | N-[((1R,4S,6R)-3-{[6-methyl-4-(2-pyrimidinyl)-2- pyridinyl]carbonyl}-3-azabicyclo[4.1.0]hept-4- yl)methyl]-5-(trifluoromethyl)-2-pyridinamine UPLC (Basic GEN_QC): rt = 0.87 minutes, peak observed: 469 (M + 1). C₂₄H₂₃F₃N₆O requires 468. ¹H NMR (500 MHz, DMSO-d₆) δ ppm 0.05-0.13 (m, 1 H) 0.71-0.82 (m, 1 H) 0.99-1.12 (m, 1 H) 1.09-1.22 (m, 1 H) 1.79-1.95 (m, 2 H) 2.50 (s, 3 H) 3.14-3.61 (m, 2 H) 3.36-3.46 (m, 1 H) 4.04-4.17 (m, 1 H) 4.37 (d, 1 H) 6.33- 6.51 (m, 1 H) 7.17-7.26 (m, 1 H) 7.47-7.78 (m, 4 H) 8.01-8.06 (m, 1 H) 8.90-8.97 (m, 2 H) |
| E32 | 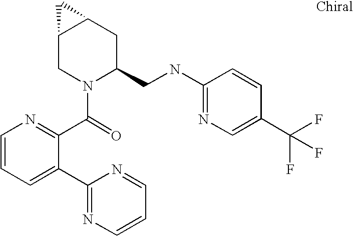 | D14 and D106 | N-[((1R,4S,6R)-3-{[3-(2-pyrimidinyl)-2- pyridinyl]carbonyl}-3-azabicyclo[4.1.0]hept-4- yl)methyl]-5-(trifluoromethyl)-2-pyridinamine UPLC (Acid IPQC): rt1 = 0.93 minutes and rt2 = 1.00 min (rotamers present), peaks observed: 455 (M + 1). C₂₃H₂₁F₃N₆O requires 454. ¹H NMR (400 MHz, DMSO-d₆) δ ppm 0.19-0.28 (m, 1 H) 0.69-0.80 (m, 1 H) 0.88-1.17 (m, 3 H) 1.71-1.84 (m, 1 H) 3.22-3.68 (m, 4 H) 4.39 (d, 1 H) 6.47 (d, 1 H) 7.34- 7.69 (m, 4 H) 8.01 -8.10 (m, 1 H) 8.42-8.59 (m, 2 H) 8.85-8.98 (m, 2 H) |
| E33 | 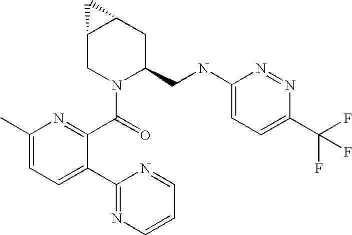 | D31 and D69 | N-[((1R,4S,6R)-3-{[6-methyl-3-(2-pyrimidinyl)-2- pyridinyl]carbonyl}-3-azabicyclo[4.1.0]hept-4- yl)methyl]-6-(trifluoromethyl)-3-pyridazinamine UPLC: (Acid Final_QC): rt = 0.67, peaks observed: 470 (M + 1). C₂₃H₂₂F₃N₇O requires 469. ¹H NMR (400 MHz, CDCl₃) δ ppm 8.92-8.80 (m, 2H) 8.55 (d, 1H) 7.95-6.78 (m, 5H) 4.78 (d, 1H) 4.54-3.13 (m, 4H) 2.63 (s, 3H) 2.42-0.44 (m, 6H). |
| E34 | 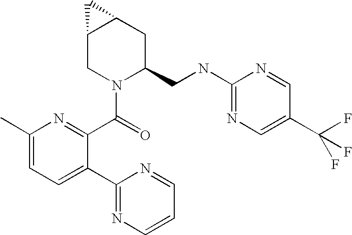 | D33 and D69 | N-[((1R,4S,6R)-3-{[6-methyl-3-(2-pyrimidinyl)-2- pyridinyl]carbonyl}-3-azabicyclo[4.1.0]hept-4- yl)methyl]-6-(trifluoromethyl)-3-pyrimidinamine UPLC: (Acid Gen_QC_SS): rt = 0.88 and 0.90 minutes (two rotamers) peaks observed: 470 (M + 1). C₂₃H₂₂F₃N₇O requires 469. ¹H NMR (400 MHz, CDCl₃) δ ppm 8.06-9.18 (m, 6 H) 7.29-7.38 (m, 2 H) 4.77 (d, 1 H) 3.05-4.30 (m, 4 H) 2.70 (s, 3 H) 0.45-2.50 (m, 6 H) |
| E35 | 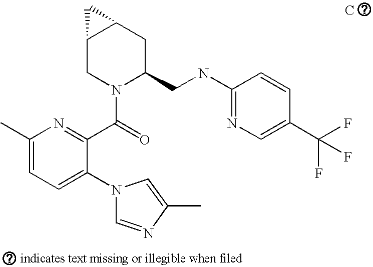 | D14 and D118 | N-[((1R,4S,6R)-3-{[6-methyl-3-(4-methyl-1H-imidazol- 1-yl)-2-pyridinyl]carbonyl}-3-azabicyclo[4.1.0]hept-4- yl)methyl]-5-(trifluoromethyl)-2-pyridinamine UPLC: (Basic GEN_QC): rt = 0.77 peak observed: 471 (M + 1). C₂₄H₂₅F₃N₆O requires 470. ¹H NMR (400 MHz, DMSO-d₆) δ ppm 7.98-8.07 (m, 1 H), 7.71-7.77 (m, 1 H), 7.68-7.70 (m, 1 H), 7.59 (dd, 1 H), 7.40-7.49 (m, 1 H), 7.30 (d, 1 H), 6.98-7.02 (m, 1 H), 6.52 (d, 1 H), 4.38 (d, 1 H), 3.34-3.55 (m, 4 H), 2.19 (s, 3 H), 2.13 (s, 3 H), 1.61-1.74 (m, 2 H), 0.79-1.09 (m, 2 H), 0.54-0.64 (m, 1 H), −0.53-−0.34 (m, 1 H) |
| E36 | 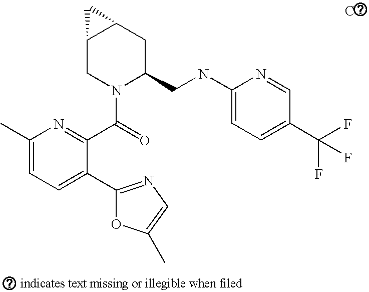 | D14 and D129 | N-[((1R,4S,6R)-3-{[6-methyl-3-(5-methyl-1,3-oxazol-2- yl)-2-pyridinyl]carbonyl}-3-azabicyclo[4.1.0]hept-4- yl)methyl]-5-(trifluoromethyl)-2-pyridinamine HPLC (walk up): rt1 = 4.46 and rt2 = 4.87 minutes ¹H NMR (500 MHz, DMSO-d₆) δ ppm 7.92-8.17 (m, 2 H), 7.24-7.71 (m, 3 H), 6.99 (s, 1 H), 6.33-6.52 (m, 1 H), 4.44 (d, 1 H), 3.42-3.79 (m, 4 H), 2.31-2.39 (m, 6 H), 1.59-1.85 (m, 2 H), 0.50-1.18 (m, 3 H), 0.21-0.35 (m, 1 H) |
| E37 | 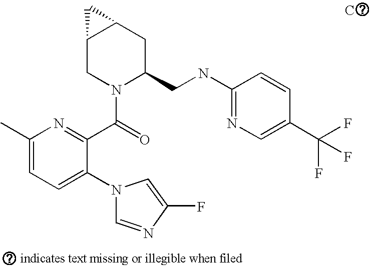 | D14 and D120 | N-[((1R,4S,6R)-3-{[3-(4-fluoro-1H-imidazol-1-yl)-6- methyl-2-pyridinyl]carbonyl}-3-azabicyclo[4.1.0]hept-4- yl)methyl]-5-(trifluoromethyl)-2-pyridinamine UPLC: (Acid QC_POS_70_900): rt = 0.72 peak observed: 475 (M + 1). C₂₃H₂₂F₄N₆O requires 474. ¹H NMR (500 MHz, DMSO-d₆) δ ppm 8.44-8.54 (m, 1 H), 8.29 (d, 1 H), 8.04-8.09 (m, 1 H), 7.99-8.01 (m, 1 H), 7.94-7.99 (m, 1H), 7.83 (d, 1 H), 7.53-7.60 (m, 1 H), 6.94-7.04 (m, 1 H), 4.85 (d, 1 H), 3.82-4.04 (m, 4 H), 2.69 (s, 3 H), 2.15-2.26 (m, 2 H), 1.48-1.57 (m, 1 H), 1.30-1.41 (m, 1 H), 1.05-1.14 (m, 1 H), −0.14-0.13 (m, 1 H) |
| E38 | 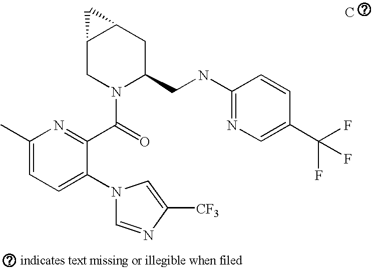 | D14 and D121 | N-{[(1R,4S,6R)-3-({6-methyl-3-[4-(trifluoromethyl)-1H- imidazol-1-yl]-2-pyridinyl}carbonyl)-3- azabicyclo[4.1.0]hept-4-yl]methyl}-5-(trifluoromethyl)- 2-pyridinamine UPLC: (Basic GEN_QC): rt = 0.89 peak observed: 525 (M + 1). C₂₄H₂₂F₆N₆O requires 524. ¹H NMR (500 MHz, DMSO-d₆) δ ppm 7.95-8.09 (m, 3 H), 7.90 (d, 1 H), 7.46-7.68 (m, 2 H), 7.40 (d, 1 H), 6.54 (d, 1 H), 4.34 (d, 1 H), 3.40-3.65 (m, 4 H), 2.23 (s, 3 H), 1.68-1.78 (m, 1 H), 1.10-1.29 (m, 1 H), 0.81-1.07 (m, 2 H), 0.47-0.61 (m, 1 H), −0.75-−0.48 (m, 1 H) |
| E39 | 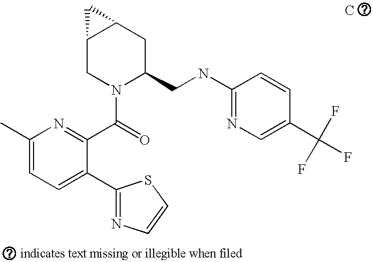 | D14 and D123 | N-[((1R,4S,6R)-3-{[6-methyl-3-(1,3-thiazol-2-yl)-2- pyridinyl]carbonyl}-3-azabicyclo[4.1.0]hept-4- yl)methyl]-5-(trifluoromethyl)-2-pyridinamine UPLC: (Basic GEN_QC): rt = 0.91 peak observed: 474 (M + 1). C₂₃H₂₂F₃N₅OS requires 473. ¹H NMR (500 MHz, DMSO-d₆) δ ppm 7.99-8.36 (m, 2 H), 7.90-7.99 (m, 1 H), 7.80-7.89 (m, 1 H), 7.48-7.61 (m, 1 H), 7.30-7.46 (m, 2 H), 6.30-6.53 (m, 1 H), 4.39 (d, 1 H), 3.34-3.58 (m, 4 H), 2.34 (s, 3 H), 1.66-1.77 (m, 2 H), 1.04-1.13 (m, 1 H), 0.90-0.99 (m, 1 H), 0.63- 0.75 (m, 1 H), 0.07-0.16 (m, 1 H). |
| E40 | 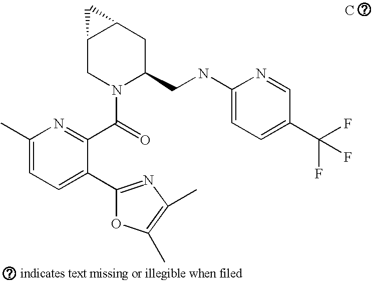 | D14 and D138 | N-[((1R,4S,6R)-3-{[3-(4,5-dimethyl-1,3-oxazol-2-yl)-6- methyl-2-pyridinyl]carbonyl}-3-azabicyclo[4.1.0]hept-4- yl)methyl]-5-(trifluoromethyl)-2-pyridinamine MS: (ES/+) m/z: 486 (M + 1). C₂₅H₂₆F₃N₅O₂ requires 485. ¹H NMR (500 MHz, DMSO-d₆) δ ppm 8.06-8.13 (m, 1 H), 7.90-8.04 (m, 1 H), 7.50-7.68 (m, 1 H), 7.36-7.41 (m, 1 H), 7.28-7.35 (m, 1 H), 6.29-6.54 (m, 1 H), 4.39- 4.47 (m, 1 H), 3.33-3.79 (m, 4 H), 2.31-2.38 (m, 3 H), 2.27 (s, 3 H), 2.06 (s, 3 H), 1.71-1.79 (m, 1 H), 1.42- 1.56 (m, 1 H), 1.07-1.15 (m, 1 H), 0.90-1.03 (m, 1 H), 0.70-0.78 (m, 1 H), 0.25-0.38 (m, 1 H) |
| E41 | 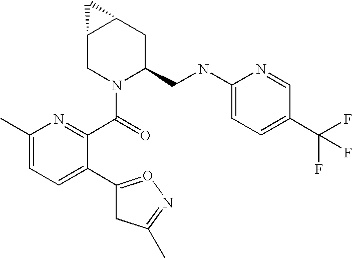 | D14 and D117 | N-[((1R,4S,6R)-3-{[6-methyl-3-(3-methyl-5- isoxazolyl)-2-pyridinyl]carbonyl}-3- azabicyclo[4.1.0]hept-4-yl)methyl]-5- (trifluoromethyl)-2-pyridinamine UPLC: (Basic GEN_QC): rt1 = 0.90 rt2 = 0.91 rotamers present, peaks observed: 472 (M + 1). C₂₄H₂₄F₃N₅O₂ requires 471. ¹H NMR (500 MHz, DMSO-d₆) δ ppm 7.84-8.06 (m, 2 H), 7.44-7.51 (m, 1 H), 7.38-7.43 (m, 1 H), 7.35 (d, 1 H), 6.48 (br. s., 1 H), 6.33 (br. s., 1 H), 4.49 (d, 1 H), 3.40-3.71 (m, 4 H), 2.52 (s, 3 H), 2.25 (s, 3 H), 1.57-1.76 (m, 2 H), 1.06-1.14 (m, 1 H), 0.92-1.00 (m, 1 H), 0.67- 0.77 (m, 1 H), 0.04-0.13 (m, 1 H) |
| E42 | 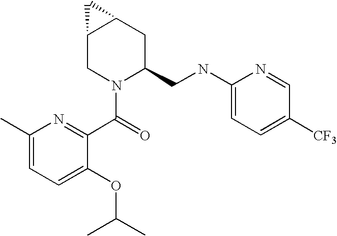 | D14 and D113 | N-{[(1R,4S,6R)-3-({6-methyl-3-[(1-methylethyl)oxy]-2- pyridinyl}carbonyl)-3-azabicyclo[4.1.0]hept-4- yl]methyl}-5-(trifluoromethyl)-2-pyridinamine MS: (ES/+) m/z: 449 (M + 1). C₂₃H₂₇F₃N₄O₂ requires 448. ¹H NMR (500 MHz, DMSO-d₆) δ ppm 0.17 (m, 1 H) 0.68 (m, 1 H) 1.15 (m, 2 H) 1.23 (m, 6 H) 1.73 (m, 2 H) 2.38 (s, 3 H) 3.33 (m, 1 H) 3.45 (m, 1 H) 3.51 (m, 2 H) 4.57 (m, 2 H) 6.49 (m, 1 H) 7.36 (m, 3 H) 7.55 (m, 1 H) 8.00 (br.s, 1 H). N-{[(1R,4S,6R)-3-({6-methyl-3-[(1-methylethyl)oxy]-2- pyridinyl}carbonyl)-3-azabicyclo[4.1.0]hept-4- yl]methyl}-5-(trifluoromethyl)-2-pyridinamine (HCl salt) UPLC (Basic GEN_QC): rt1 = 0.93 and rt2 = 0.94 minutes, rotamers present, peaks observed: 449 (M + 1 − HCl). C₂₃H₂₇F₃N₄O₂•HCl requires 485. |

### Example 43

### 6-{[((1R,4S,6R)-3-{[6-methyl-3-(2-pyrimidinyl)-2-pyridinyl]carbonyl}-3-azabicyclo[4.1.0]hept-4-yl)methyl]amino}-4-(trifluoromethyl)-3-pyridinecarbonitrile (E43)

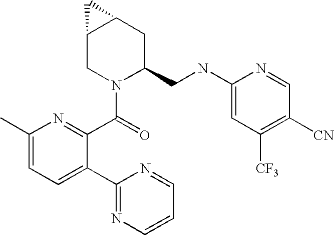

2-chloro-6-{[((1R,4S,6R)-3-{[6-methyl-3-(2-pyrimidinyl)-2-pyridinyl]carbonyl}-3-azabicyclo[4.1.0]hept-4-yl)methyl]amino}-4-(trifluoromethyl)-3-pyridinecarbonitrile D111 (30 mg), palladium(II) acetate (1.276 mg, 5.68 μmol), triphenylphosphine (5.96 mg, 0.023 mmol), K₂CO₃ (15.71 mg, 0.114 mmol) were collected and shaken at 50° C. overnight. A few drops of 1 M HCl was added, and then concentrated under vacuum. The resulting crude was purified with Biotage SP1, over a 50 g SNAP C18 column, eluting with a gradient of ACN and water (modified with 0.5% HCOOH). Fractions containing the required product were collected and neutralised with a 1 g SCX column to give the title compound E43 as colourless solid (15 mg). C₂₅H₂₂F₃N₇O requires 494. ¹H NMR (500 MHz, DMSO-d₆): 9.00-8.76 (m, 2H), 8.74-8.20 (m, 3H), 7.61-7.26 (m, 2H), 6.85-6.74 (s, 1H), 4.43-4.26 (m, 1H), 3.91-3.38 (m, 4H), 2.40-2.31 (s, 3H), 1.82-1.46 (m, 2H), 1.18-0.92 (m, 2H), 0.80-0.71 (m, 1H), 0.29-0.16 (m, 1H).

### Example 44

### 3-fluoro-N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyridinamine (E44)

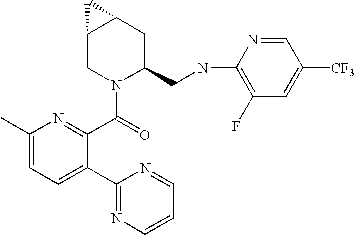

To a mixture of [((1R,4S,6R)-3-{[6-methyl-3-(2-pyrimidinyl)-2-pyridinyl]carbonyl}-3-azabicyclo[4.1.0]hept-4-yl)methyl]amine D25 (50 mg) and potassium carbonate (42.7 mg, 0.309 mmol) in dry DMF (1.5 ml), a solution of 2,3-difluoro-5-(trifluoromethyl)pyridine (34.0 mg, 0.186 mmol) in DMF (0.5 ml) was added and the suspension was shacken at 70° C. in a screw-capped vial for 1 hour. After cooling mixture was diluted with AcOEt and washed with water and brine. Organics were dried and evaporated, and the crude was purified by flash chromatography (KP-Sil SNAP 10 g eluting with Cy/AcOEt 1:1) affording the title compound E44 (53 mg). UPLC (Acid GEN_QC SS): rt=0.96, peak observed: 487 (M+1). C₂₄H₂₂F₄N₆O requires 486. ¹H NMR (500 MHz, DMSO-d₆) δ ppm 8.51-8.65 (m, 2H), 8.11 (d, 1H), 7.68 (br. s., 1H), 7.41 (d, 1H), 7.30 (br. s., 1H), 7.16-7.21 (m, 1H), 7.11 (d, 1H), 4.13 (d, 1H), 3.46-3.69 (m, 2H), 3.07-3.13 (m, 2H), 2.15 (s, 3H), 1.28-1.56 (m, 2H), 0.81 (br. s., 1H), 0.75 (d, 1H), 0.48 (d, 1H), 0.00 (d, 1H).

### Example 45

### N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyrazinamine (E45)

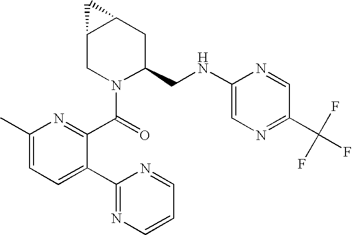

[((1R,4S,6R)-3-{[6-methyl-3-(2-pyrimidinyl)-2-pyridinyl]carbonyl}-3-azabicyclo[4.1.0]hept-4-yl)methyl]amine D25 (80 mg) and 2-bromo-5-(trifluoromethyl)pyrazine (67.4 mg, 0.297 mmol) were dissolved in DMF (2 ml) then sodium carbonate (52.4 mg, 0.495 mmol) was added and the mixture was heated to 50° C. for 2 hours. DMF was evaporated under vacuum and the residue was dissolved in DCM (4 ml) and washed with NaHCO₃ saturated solution (4 ml). The organic phase was filtered through a phase separator tube, concentrated under vacuum and the resulting crude product was purified by SCX Chromatography (column size 5 g). Another purification was performed by silica —NH chromatography (Biotage SP—column size 25 g using Cy:EtOAc=5:5 to EtOAc as eluent). It was recovered the title compound E45 (30 mg). UPLC: (Acid Final_QC): rt=0.78 and 0.79 minutes (two rotamers), peaks observed: 470 (M+1). C₂₃H₂₂F₃N₇O requires 469. ¹H NMR (400 MHz, DMSO-d₆) δ ppm 8.91-8.82 (m, 2H), 8.36 (d, 1H), 8.20-7.86 (m, 3H), 7.47 (t, 1H), 7.36 (d, 1H) 4.40 (d, 1H), 3.81-3.55 (m, 2H), 3.49-3.35, (m, 2H), 2.38-2.30 (br. s., 3H), 1.80-1.65 (m, 2H), 1.15-1.06 (m, 1H), 1.03-0.91 (m, 1H), 0.80-0.71 (m, 1H), 0.29-0.19 (m, 1H).

### Example 46

### N-[((1R,4S,6R)-3-{[3-methyl-6-(2-pyrimidinyl)-2-pyridinyl]carbonyl}-3-azabicyclo[4.1.0]hept-4-yl)methyl]-5-(trifluoromethyl)-2-pyridinamine (E46)

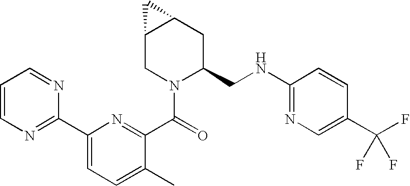

3-methyl-6-(2-pyrimidinyl)-2-pyridinecarboxylic acid HCl salt D110 (55.7 mg) was treated with DCM (1 ml) and TEA (3 drops) and evaporated to dryness to remove the NH₄Cl. To the resulting solid under Argon, dry DCM (2 ml) was added followed by pentafluorophenol (40.7 mg, 0.221 mmol) and N,N′-dicyclohexylcarbodiimide (45.6 mg, 0.221 mmol). The heterogeneous slurry was stirred at room temperature for 4 hours. N-[(1R,4S,6R)-3-azabicyclo[4.1.0]hept-4-ylmethyl]-5-(trifluoromethyl)-2-pyridinamine D14 (50 mg) was then added followed by TEA (0.051 ml, 0.369 mmol). The reaction mixture was stirred at room temperature overnight. The reaction mixture was taken up with DCM (4 ml) and filtered. The eluted DCM was treated with NaHCO₃ saturated solution (3 ml) and brine. Evaporation of the organic solvent afforded crude material, (160 mg) as yellow solid that was purified by preparative LCMS (AA_Prep_Purification). The solution was recovered from preparative LCMS, evaporated and the residue treated with water (30 ml)/DCM (50 ml). The phases were separated and the aqueous phase was back extracted with DCM (2×50 ml). The combined organics were dried over Na₂SO₄ and evaporated to dryness to get, after standing under high vacuum overnight, the title compound E46 (40 mg) as white solid. MS: (ES/+) m/z: 469 (M+1). C₂₄H₂₃F₃N₆O requires 468. ¹H NMR (400 MHz, DMSO-d₆) δ ppm 0.07 (m, 1H) 0.78 (m, 1H) 1.02 (m, 1H) 1.15 (m, 1H) 1.79 (m, 2H) 2.14 (s, 3H) 3.13 (m, 1H) 3.25 (m, 1H) 3.72 (m, 1H) 3.91 (m, 1H) 4.5 (d, 1H) 6.77 (m, 1H) 7.56 (m, 2H) 7.75 (m, 2H) 8.07 (m, 1H) 8.32 (d, 1H) 8.97 (d, 2H).

### Example 47

### N-[((1R,4S,6R)-3-{[6-methyl-3-(5-methyl-1,3-oxazol-2-yl)-2-pyridinyl]carbonyl}-3-azabicyclo[4.1.0]hept-4-yl)methyl]-5-(trifluoromethyl)-2-pyrimidinamine (E47)

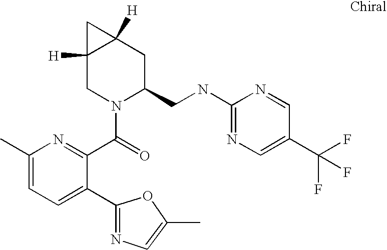

In a 8 ml screw cap vial 6-methyl-3-(5-methyl-1,3-oxazol-2-yl)-2-pyridinecarboxylic acid D129 (15 mg) was dissolved in DMF (0.5 ml), to the solution DIPEA (0.048 ml, 0.275 mmol) and TBTU (30.9 mg, 0.096 mmol) were added sequentially and the resulting mixture was stirred for 30 min at room temperature. After this time a solution of N-[(1R,4S,6R)-3-azabicyclo[4.1.0]hept-4-ylmethyl]-5-(trifluoromethyl)-2-pyrimidinamine D33 (18.72 mg) in DMF (1.5 ml) was added to the reaction mixture and the stirring was maintained for 1.5 hours. A saturated solution of NaHCO₃ (2 ml) was added and the mixture was evaporated under reduced pressure to give a brown solid which was dissolved with EtOAc (4 ml) and then filtered. The organic solvent was removed under vacuum and the brown oil obtained was purified by column chromatography on silica gel (Biotage NH 25+M; eluted with Cy/EtOAc: 8 CV 1/0 to 7/3, 12 CV 7/3). The fractions were collected and evaporated to give a pale yellow solid, the title compound E47 (11 mg). UPLC (Basic GEN_QC): rt1=0.89 minutes and rt2=0.95 (rotamers present), peaks observed: 473 (M+1). C₂₃H₂₃F₃N₆O₂ requires 472.

### N-[((1R,4S,6R)-3-{[6-methyl-3-(5-methyl-1,3-oxazol-2-yl)-2-pyridinyl]carbonyl}-3-azabicyclo[4.1.0]hept-4-yl)methyl]-5-(trifluoromethyl)-2-pyrimidinamine hydrochloride

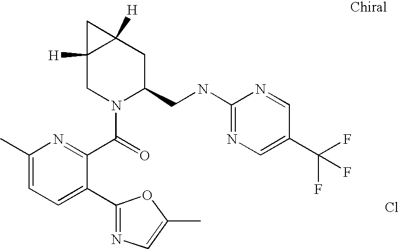

To an ice cooled solution of N-[((1R,4S,6R)-3-{[6-methyl-3-(5-methyl-1,3-oxazol-2-yl)-2-pyridinyl]carbonyl}-3-azabicyclo[4.1.0]hept-4-yl)methyl]-5-(trifluoromethyl)-2-pyrimidinamine (10.3 mg, 0.022 mmol) in DCM (0.5 ml) was added HCl (1M in diethylether) (0.044 mL, 0.044 mmol) and stirred at room temperature. After 1 hour the solvents were removed under vacuum and the sticky pale yellow solid obtained was triturated with anhydrous Et₂O (0.7 ml), then it was removed by suction to give a white powdery solid, the title compound (9 mg). UPLC (Basic GEN_QC): rt1=0.89 minutes and rt2=0.95 (rotamers present), peaks observed: 473 (M+1−HCl). C₂₃H₂₃F₃N₆O₂HCl requires 508.

### Example 48

### N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyridinamine

A larger scale synthesis for the compound of example 7 is described here as example 48. The synthesis is in 5 stages.

### Stage 1

### 1,1-dimethylethyl (1R,6R)-2-oxo-3-azabicyclo[4.1.0]heptane-4-carboxylate

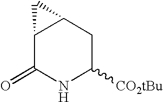

Sodium iodide (391 g, 2.6 mol, 1.5 eq) was partially dissolved in acetonitrile (1.7 L) after stirring at 20° C. for 10 min under nitrogen atmosphere. TMS-Cl (0.323 L, 2.5 mol, 1.5 eq) was added over 10 minutes and the resulting yellow slurry was stirred at 20° C. for 1 hour. A solution of (1R,5S)-3-oxabicyclo[3.1.0]hexan-2-one (Minakem supplier, 170 g, 1.73 mol, 1 eq) in acetonitrile (340 mL) was added over 5 minutes at 20° C. The suspension was heated to 50° C. (internal temperature), then kept for 3 hour 45 minutes at 50° C. The mixture was diluted with methanol (1.7 L) at 20° C. and concentrated to 5 volumes (850 ml) under reduced pressure. Methanol (1.7 L) was then added followed by TMS-Cl (0.102 L, 0.8 mol, 0.5 eq). The resulting mixture was stirred at 20° C. for 15 hours 30 minutes. The mixture was concentrated under vacuum to 5 volumes (0.85 L), then 2-MeTHF was added (1.7 L) and the solution was concentrated to 5 volumes (0.85 L). 2-MeTHF was added (1.7 L). The dark red solution was washed with aqueous Na₂SO₃ 20% w/w (0.68 L) at 20° C. (solution became colourless-light yellow). The biphasic system was separated and the organic layer was washed with water (0.68 L), then concentrated under vacuum to 4 volumes (0.68 L). 2-MeTHF (1.7 L) was added and the solution of methyl (1R,2S)-2-(iodomethyl)cyclopropanecarboxylate was concentrated to 5 volumes (0.85 L), diluted with 2-Me-THF (0.51 L).

N-(Diphenylmethylene)glycine t-butylester (503.2 g, 1.7 mol, 1.2 eq) was suspended in dry Me-THF (1.7 L) at 20° C. under nitrogen. The mixture was cooled to 0° C. and KOtBu (195.5 g, 1.74 mol, 1 eq) was added in three portions. The slurry was become a yellow-orange solution and was stirred at 0° C. for 30 minutes. The previous solution of methyl (1R,2S)-2-(iodomethyl)cyclopropanecarboxylate in 2-MeTHF was slowly added over 25 minutes, keeping the temperature lower than 5° C. during the addition. The mixture was stirred at 0° C. for 2.5 hours. The mixture was quenched with buffer pH=7 (KH₂PO₄/Na₂HPO₄) (340 ml) at 0° C. The biphasic system was warmed at 20° C. The water phase was discharged. To the organic phase at 0° C. was added citric acid 30% w/w (1.36 L) keeping the temperature 0-5° C. and the biphasic system was stirred for 16 hours 20 minutes at 20° C. Cyclohexane (3.4 L) was added and the phases were separated. The water phase was washed with cyclohexane (3.4 L). Ethyl acetate (3.4 L) was added to the water phase, and then the system was basified to pH=8.5 with aqueous saturated K₂CO₃ (0.85 L) then diluted with water (0.425 L). The biphasic system was separated. The aqueous layer was back extracted with ethyl acetate (3.4 L). The combined organic phases were washed with water (0.51 L), concentrated to 10 volumes (1.7 L). Toluene (3.4 L) was added and the solution was concentrated to 10 volumes (1.7 L), diluted again with toluene (0.85 L). To this solution was added HCl 37% (0.85 ml, catalytic amount). The solution was heated to 105° C. for 20 hours. The solution was cooled at 40° C., reduced to 4 volumes (0.68 L) under reduced pressure and heptane (1.19 L) was added over 1 hour. The mixture was stirred at 40° C. for 30 minutes and then cooled at 15° C. over 1 hour: a solid was precipitated. The slurry was stirred at 15° for approximately 16 hours and then filtered. The solid was washed with heptane (2×0.425 L), dried in a vacuum oven at 40° C. for 20 hours and 30 minutes. 1,1-dimethylethyl (1R,6R)-2-oxo-3-azabicyclo[4.1.0]heptane-4-carboxylate (syn/anti mixture, 194 g) was obtained as white solid.

¹H NMR (600 MHz, DMSO-d6) δ ppm 6.86-7.39 (1H, 2 m), 3.81 (1H, 2 dd), 2.20-2.33 (1H, 2 m), 1.74-2.11 (1H, 2 m), 1.42 (9H, s), 1.4-1.6 (1H, m), 0.90-1.12 (1H, 2 m), 0.69-0.88 (1H, 2 m)

### Stage 2

### 1,1-dimethylethyl(1R,4S,6R)-4-(hydroxymethyl)-3-azabicyclo[4.1.0]heptane-3-carboxylate

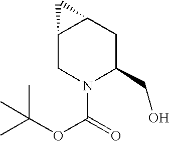

The 1,1-dimethylethyl (1R,6R)-2-oxo-3-azabicyclo[4.1.0]heptane-4-carboxylate, (150 g, 1 eq) was dissolved in toluene (0.450 L) and MeOH (1.05 L) stirred for 5 minutes at 20° C. The temperature was cooled to 15° C. and KOH (60 g, 1.06 mol, 1.5 eq) was added in two portions. The solution was stirred at 20° C. for 3 hours. The solution was cooled to 10° C., TMSCl (0.36 L, 2.84 mol, 4 eq) was added keeping the temperature around 10-15° C. over 40 minutes. White solid was precipitated (KCl). The slurry was stirred at room temperature overnight. The pH of the organic phase was measured and found to be 1. NaHCO₃ solid (240 g) was added in four portions to reach pH=5.5. The volume was reduced to 4 volumes (0.6 L). THF (1.5 L) was added and the volume is reduced to 4 volumes (0.6 L) by distillation under reduced pressure. The solid was filtered (note: 60 ml of the slurry was collected prior to filtration, so 10% of input was removed) and washed with THF (3×0.3 L). The filtrate appeared cloudy. The solution was reduced to 2.2 volumes (0.337 L) by distillation under reduced pressure and BF₃.THF (422.55 mL, 3.83 mol, 6 eq considering the 10% removed) was added under stirring whilst maintaining an internal temperature of 25° C. The resulting solution was added slowly to a solution of LiBH₄ (4M in THF) (0.648 L, 2.59 mol, 4 eq) diluted with THF (0.405 L) keeping the temperature at 25-30° C. (the line was washed with THF (0.337 L)). The mixture was stirred at 30° C. overnight (17 hours). The mixture was quenched slowly with MeOH (0.54 L) at 25-30° C. The solution was stirred at 50° C. for approximately 1 hour. After this time, the solution was reduced to 5.5 volumes (742.5 mL) by distillation under reduced pressure. HCl 3M (0.540 L) was then added at 10-15° C. The mixture was stirred at 20° C. for 1 hour and toluene (0.54 L) was added. The phases were separated. The aqueous phase was washed with toluene (3×0.54 L). The aqueous layer was basified with 6M NaOH (405 mL) until pH=9. To the basic aqueous solution at 25° C. were successively added THF (67.5 mL) and a solution of ditert-butyl dicarbonate in THF (50% wt/vol, d=0.92, 0.25 L, 0.626 mol, 0.93 eq) The pH was adjusted to pH=8.5 by addition of 6M NaOH (0.135 L). The resulting slurry was stirred for 30 minutes at 25° C. and the pH was adjusted to pH=9 by addition of 6M NaOH (0.135 L). The slurry was then stirred for 3 hours and then filtered. The inorganic salts were washed with MTBE (2×0.27 L). The filtrate was diluted with MTBE (1.08 L). The biphasic system was separated. The organic phase was washed with NaCl 20% w/w (0.54 L) and then concentrated under reduced pressure to 2.5 volumes (337.5 mL). Heptane (1.35 L) was added and the solution was reduced to 5 volumes (0.675 L), diluted with heptane (0.675 L) and concentrated to 5 volumes (0.675 L) by distillation under reduced pressure. Seed (135 mg) of the title compound was added at 40° C. and the slurry was cooled at 20° C. in 1 hour. The slurry was stirred for at least 4 hours and filtered. The solid was washed with cold heptane (0.27 L) and dried in a vacuum oven at 40° C. for 14 hours and 30 minutes. 1,1-dimethylethyl(1R,4S,6R)-4-(hydroxymethyl)-3-azabicyclo[4.1.0]heptane-3-carboxylate (98 g) was obtained as white solid. ¹H NMR (600 MHz, DMSO-d6) δ ppm 4.67 (1H, br. s.), 3.6-3.9 (2H, m), 3.2-3.5 (3H, m), 1.89 (1H, m), 1.54 (1H, m), 1.37 (9H, br. s.), 0.90 (2H, m), 0.58 (1H, m), −0.09 (1H, q)

### Stage 3

### 1,1-dimethylethyl (1R,4S,6R)-4-(bis{[5-(trifluoromethyl)-2-pyridinyl]amino}methyl)-3-azabicyclo[4.1.0]heptane-3-carboxylate

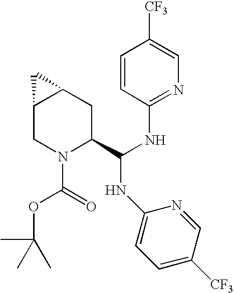

In a vessel, 1,1-dimethylethyl(1R,4S,6R)-4-(hydroxymethyl)-3-azabicyclo[4.1.0]heptane-3-carboxylate (200 g, 1 eq) was dissolved in ethyl acetate (0.4 L) and triethylamine (0.49 L, 3.5 mol, 4 eq) and the resulting solution was cooled down to 10° C. In a second vessel, sulfur trioxide pyridine complex (276 g, 1.73 mol, 1.97 eq) was dissolved at 20° C. in dimethylsulfoxide (1.2 L) and the resulting solution was added dropwise in the first vessel for 40 minutes keeping the internal temperature below 15° C. The reaction mixture was stirred at 10° C. for 35 minutes. Water (1 L) was carefully added dropwise over 35 minutes at 13° C. to quench the mixture, maintaining the internal temperature below 15° C. (quench was exothermic). The quenched reaction mixture was purged with nitrogen for 1 hour 30 minutes while the evolved gas dimethylsulfide was scrubbed with aqueous NaClO. Ethyl acetate (1.6 L) was added to extract the aldehyde, the aqueous layer was discharged. The organic layer was washed with citric acid aq 10% w/w (2×1 L), with NaCl aq 10% w/w (1 L). The organic layer was concentrated under vacuum to 3 volumes (0.6 L), CH₃CN (1.2 L) was added and the solution of the aldehyde was concentrated again to 3 volumes (0.6 L).

To this solution, 5-(trifluoromethyl)-2-pyridinamine (340 g, 2.09 mol, 2.38 eq) was added and followed by acetic acid (0.2 L, 3.49 mol, 3.97 eq) and more CH₃CN (0.6 L). The resulting solution was stirred at 20° C. overnight. Water (2 L) was added at 20° C. to complete the precipitation and the resulting suspension was stirred for 2 hours and 20 minutes at 20° C. The slurry was filtered and the wet cake was washed twice with a mixture CH₃CN/water 1:4 (2×0.6 L), dried in the oven at 40° C. for at least 16 hours. 1,1-dimethylethyl (1R,4S,6R)-4-(bis{[5-(trifluoromethyl)-2-pyridinyl]amino}methyl)-3-azabicyclo[4.1.0]heptane-3-carboxylate (368 g) was obtained as white solid.

¹H NMR (600 MHz, Acetone-d6) δ ppm 8.34 (2H, m), 7.67 (2H, m), 7.0-7.2 (1H, m), 6.6-6.9 (3H, m), 6.31 (1H, m), 4.4-4.7 (1H, m), 3.72-4.00 (1H, 2d), 3.32-3.47 (1H, 2d), 2.22 (1H, m), 1.71 (1H, m), 1.41 (9H, s), 1.05 (2H, m), 0.68 (1H, m), −0.07 (1H, m)

### Stage 4

### 1,1-dimethylethyl (1R,4S,6R)-4-({[5-(trifluoromethyl)-2-pyridinyl]amino}methyl)-3-azabicyclo[4.1.0]heptane-3-carboxylate

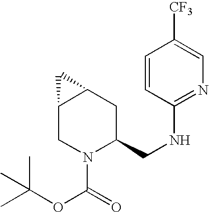

To a solution suspension of 1,1-dimethylethyl (1R,4S,6R)-4-(bis{[5-(trifluoromethyl)-2-pyridinyl]amino}methyl)-3-azabicyclo[4.1.0]heptane-3-carboxylate (300 g, 1 eq) in THF (1.05 L) was added sodium triacetoxyborohydride (600 g, 2.83 mol, 5.05 eq) was added portion wise (at least 5 portions) whilst maintaining the temperature below 25° C. Acetic acid (0.45 L, 4.4 mol, 7.86 eq) was then added at 15° C. The mixture was heated gently to 40° C. and stirred for 4 hours and 45 minutes. After cooling to 10° C. over 30 minutes, water (3 L) was added and the quenched mixture was warmed to 20° C. The seed (300 mg-0.001 wt) was then added. The resulting slurry was stirred for approximately 17 hours at 20° C. and then filtered. The solid was washed with a mixture THF/water 1:4 (2×900 ml), dried in the vacuum oven at 40° C. for 22 hours. 1,1-dimethylethyl (1R,4S,6R)-4-({[5-(trifluoromethyl)-2-pyridinyl]amino}methyl)-3-azabicyclo[4.1.0]heptane-3-carboxylate (178 g) was obtained as white solid.

¹H NMR (600 MHz, DMSO-d6) δ ppm 8.28 (1H, br. s.), 7.61 (1H, d), 7.3-7.5 (1H, m), 6.59 (1H, d), 4.0-4.3 (1H, m), 3.6-3.9 (1H, m), 3.2-3.5 (4H, m), 1.83 (1H, m), 1.59 (1H, m), 1.34-1.14 (9H, 2s), 0.96 (2H, m), 0.63 (1H, dt), −0.13 (1H, m)

### Stage 5

### N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyridinamine

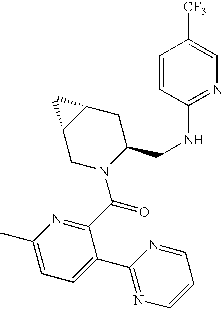

A) To a suspension of 1,1-dimethylethyl (1R,4S,6R)-4-({[5-(trifluoromethyl)-2-pyridinyl]amino}methyl)-3-azabicyclo[4.1.0]heptane-3-carboxylate (150 g, 1 eq.) in DCM (300 ml) at 25° C. was added 6M HCl (0.75 L) dropwise. The mixture was stirred vigorously at 25° C. for 5 hours, cooled to 10° C. and basified with 6M NaOH (0.75 L) over 15 min (pH of approximately 12). DCM (1.2 L) was added. The biphasic system was stirred vigorously for 5 minutes and separated. The aqueous layer was back extracted with DCM (0.75 L). The combined organic layers were washed with water (0.75 L). The organic solution of N-[(1R,4S,6R)-3-azabicyclo[4.1.0]hept-4-ylmethyl]-5-(trifluoromethyl)-2-pyridinamine was concentrated to 3 vol (0.45 L) at atmospheric pressure.

B) To a suspension of 6-methyl-3-(2-pyrimidinyl)-2-pyridinecarboxylic acid (Manchester Organics, approximately 65% wt pure, 147 g, mol 1.1 eq) in DCM (0.54 L) at 22° C. was added a solution of pentafluorophenol (PFP, 82.5 g, mol, 1.1 eq) in DCM (0.27 L) over 5 minutes, followed by a solution of dicyclohexylcarbodiimide (DCC, 91.5 g, mol, 1.1 eq) in DCM (0.27 L) over 15 minutes. The resulting mixture was stirred at 22° C. for 3 hours. The previous solution of N-[(1R,4S,6R)-3-azabicyclo[4.1.0]hept-4-ylmethyl]-5-(trifluoromethyl)-2-pyridinamine (0.45 L) was then added, followed by triethylamine (109.5 mL, 0.79 mol, 2 eq) added over 14 minutes. The resulting suspension was stirred at 22° C. for at least 20 hours. The mixture was filtered. The solid was washed with DCM (2×0.225 L). The filtrates were collected and the resulting organic solution was washed with 1N HCl (0.525 L), then with 1N NaOH (0.525 L) and water (0.525 L) and then concentrated down to 3 volumes (0.45 L). 2-Propanol (1.05 L) was added. The mixture was concentrated down to 5 volumes (0.75 L). 2-Propanol (0.75 L) was added and the mixture was warmed at reflux (81° C.) to obtain a clear solution. Then the solution was cooled down to 22° C. over 30 minutes and then stirred for approximately 17 hours. The solid was filtered and washed with IPA (2×0.225 L) and dried at 40° C. in vacuo for 6.5 hours. The title compound, N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyridinamine, (146 g) was obtained as white solid.

¹H NMR (600 MHz, METHANOL-d4) δ ppm 8.87 (1H, d), 8.55 (1H, d), 8.05 (1H, br. s.), 7.52 (1H, d), 7.41 (1H, m), 6.49 (1H, d), 4.57 (1H, d), 3.82 (2H, m), 3.43 (1H, dd), 2.55 (2H, s), 1.85 (2H, m), 1.0-1.2 (2H, m), 0.87 (1H, td, 4.4 Hz), 0.43 (1H, q)

[This procedure was also performed on 5 g scale of 1,1-dimethylethyl (1R,4S,6R)-4-({[5-(trifluoromethyl)-2-pyridinyl]amino}methyl)-3-azabicyclo[4.1.0]heptane-3-carboxylate and the resulting N-[(1R,4S,6R)-3-azabicyclo[4.1.0]hept-4-ylmethyl]-5-(trifluoromethyl)-2-pyridinamine (3.6 g) was isolated. The stereochemistry has been proven via NOESY experiment].

The scheme for the synthesis of example 48 is shown below as scheme 4.

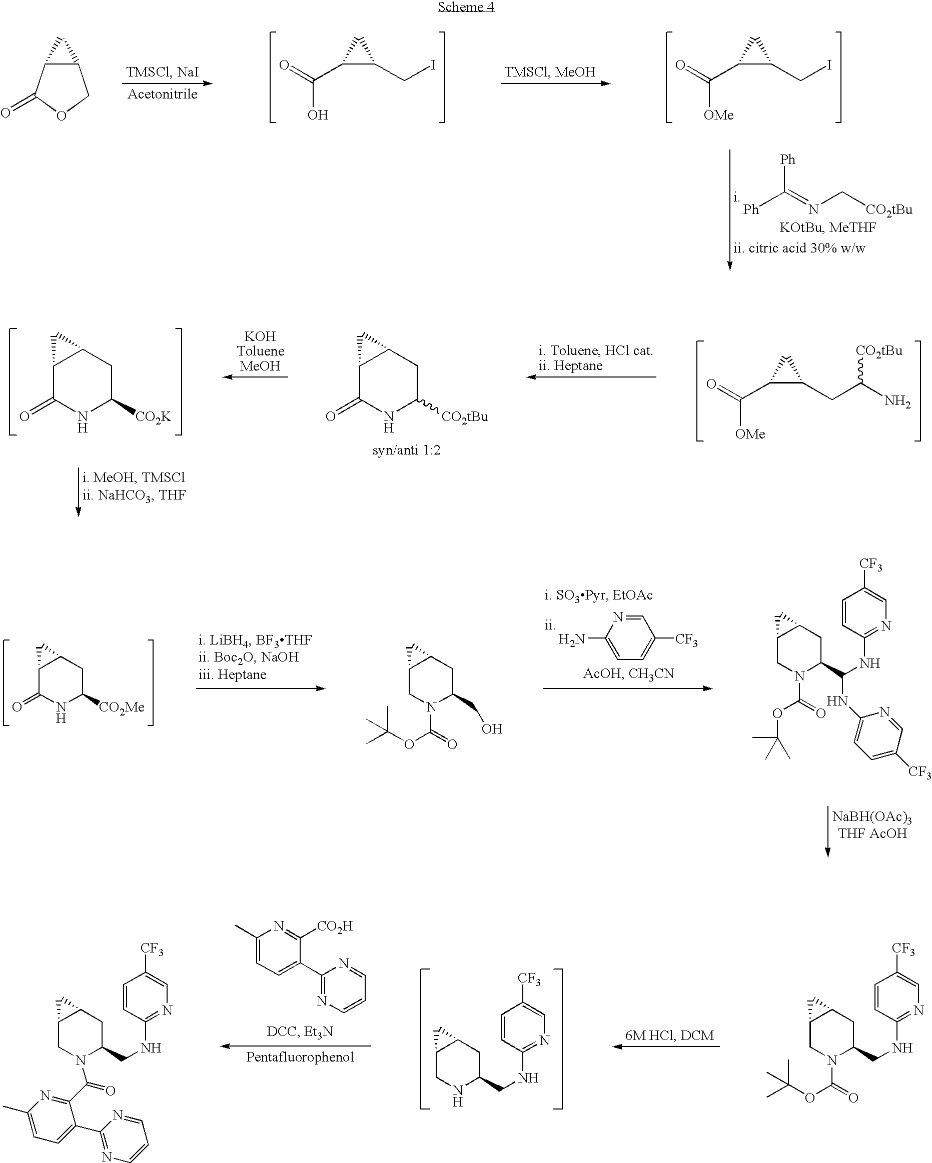

Examples 49 to 59 were made using methods similar to those described above for examples 1 to 47.

### Example 49

### N-[((1R,4S,6R)-3-{[6-methyl-3-(5-methyl-1,3,4-oxadiazol-2-yl)-2-pyridinyl]carbonyl}-3-azabicyclo[4.1.0]hept-4-yl)methyl]-5-(trifluoromethyl)-2-pyridinamine

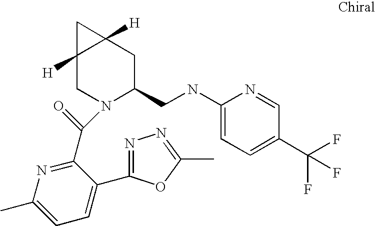

| No. | | Reactants | Characterising data |
|---|---|---|---|
| E50 | 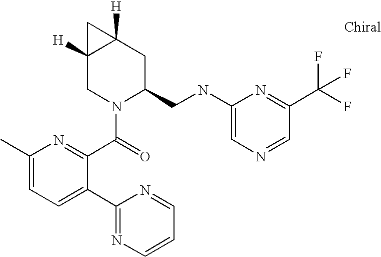 | D25 and 2-chloro-6- (trifluoromethyl) pyrazine | N-[((1R,4S,6R)-3-{[6-methyl-3-(2-pyrimidinyl)-2- pyridinyl]carbonyl}-3-azabicyclo[4.1.0]hept-4- yl)methyl]-6-(trifluoromethyl)-2-pyrazinamine MS: (ES/+) m/z: 470 (M + 1). C₂₃H₂₂F₃N₇O requires 469. ¹H NMR (500 MHz, DMSO-d₆) δ ppm 8.84-8.91 (m, 2 H), 8.32-8.36 (m, 1 H), 7.99-8.16 (m, 2 H), 7.76- 7.83 (m, 1 H), 7.47 (t, 1 H), 7.36 (d, 1 H), 4.42 (d, 1 H), 3.57-3.76 (m, 3 H), 3.37-3.41 (m, 1 H), 2.38 (s, 3 H), 1.60-1.81 (m, 2 H), 1.06-1.15 (m, 1 H), 0.97-1.03 (m, 1 H), 0.71-0.80 (m, 1 H), 0.21-0.29 (m, 1 H) |

| | | Amide coupling | |
|---|---|---|---|
| No. | | Reactants | Characterising data |
| E51 | 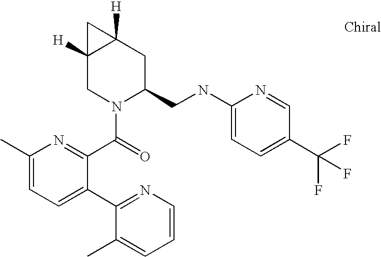 | D14 and 3,6′- dimethyl-2,3′- bipyridine-2′- carboxylic acid (synthesis similar to D93) | N-({(1R,4S,6R)-3-[(3,6′-dimethyl-2,3′-bipyridin-2′- yl)carbonyl]-3-azabicyclo[4.1.0]hept-4-yl}methyl)-5- (trifluoromethyl)-2-pyridinamine ¹H NMR (400 MHz, DMSO-d₆) δ ppm 8.44 (d, 1 H), 8.20 (s, 1 H), 7.68-7.82 (m, 2 H), 7.63 (dd, 1 H), 7.54 (t, 1 H), 7.22-7.41 (m, 2 H), 6.58 (d, 1 H), 4.19 (d, 1 H), 3.67-3.78 (m, 1 H), 3.55-3.68 (m, 1 H), 3.31- 3.45 (m, 1 H), 3.09-3.16 (m, 1 H), 2.40 (s, 3 H), 2.19 (s, 3 H), 1.67-1.83 (m, 2 H), 0.85-1.06 (m, 2 H), 0.50-0.59 (m, 1 H), −0.42-−0.31 (m, 1 H) |
| E52 | 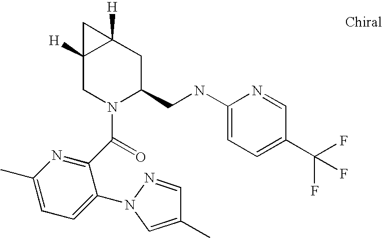 | D14 and 6-methyl-3- (4-methyl-1H- pyrazol-1-yl)-2- pyridinecarboxylic acid (synthesis simliar to D71) | N-[((1R,4S,6R)-3-{[6-methyl-3-(4-methyl-1H- pyrazol-1-yl)-2-pyridinyl]carbonyl}-3- azabicyclo[4.1.0]hept-4-yl)methyl]-5- (trifluoromethyl)-2-pyridinamine MS: (ES/+) m/z: 471 (M + 1). C₂₄H₂₅F₃N₆O requires 470. ¹H NMR (500 MHz, DMSO-d₆) δ ppm 8.06 (br. s., 1 H), 7.85 (d, 1 H), 7.77 (s, 1 H), 7.52-7.62 (m, 2 H), 7.40-7.46 (m, 1 H), 7.30 (d, 1 H), 6.51 (d, 1 H), 4.40 (d, 1 H), 3.50 (d, 2 H), 3.28 (d, 2 H), 2.14-2.35 (m, 3 H), 2.07 (s, 3 H), 1.51-1.74 (m, 2 H), 1.01-1.10 (m, 1 H), 0.84-0.95 (m, 1 H), 0.60-0.69 (m, 1 H), −0.18- 0.00 (m, 1H) |

### Example 53

### N-[((1R,4S,6R)-3-{[5-methyl-6-(2-pyrimidinyl)-2-pyridinyl]carbonyl}-3-azabicyclo[4.1.0]hept-4-yl)methyl]-5-(trifluoromethyl)-2-pyridinamine

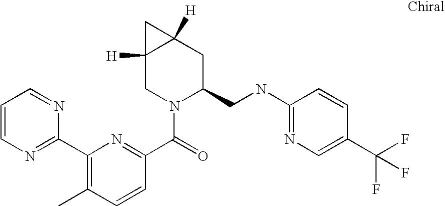

### Example 54

### N-{[(1R,4S,6R)-3-({3-[(cyclopropylmethyl)oxy]-6-methyl-2-pyridinyl}carbonyl)-3-azabicyclo[4.1.0]hept-4-yl]methyl}-5-methyl-2-pyridinamine hydrochloride

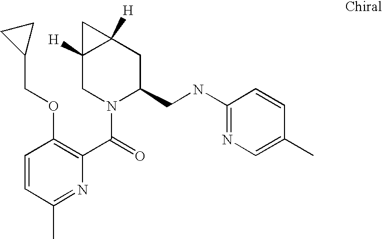

### Example 55

### N-[((1R,4S,6R)-3-{[3-(ethyloxy)-6-methyl-2-pyridinyl]carbonyl}-3-azabicyclo[4.1.0]hept-4-yl)methyl]-5-methyl-2-pyrimidinamine hydrochloride

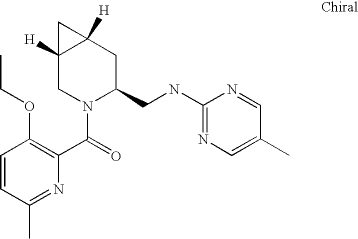

### Example 56

### N-[((1R,4S,6R)-3-{[3-(ethyloxy)-6-methyl-2-pyridinyl]carbonyl}-3-azabicyclo[4.1.0]hept-4-yl)methyl]-5-(trifluoromethyl)-2-pyrimidinamine hydrochloride

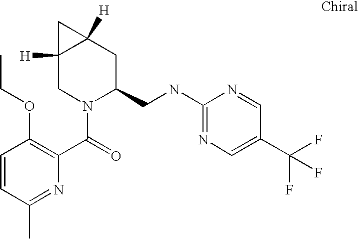

### Example 57

### N-[((1R,4S,6R)-3-{[6-methyl-3-(propyloxy)-2-pyridinyl]carbonyl}-3-azabicyclo[4.1.0]hept-4-yl)methyl]-5-(trifluoromethyl)-2-pyrimidinamine hydrochloride

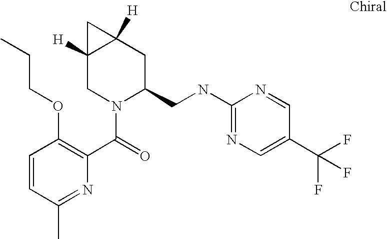

### Example 58

### 5,6-dimethyl-N-[((1R,4S,6R)-3-{[6-methyl-3-(propyloxy)-2-pyridinyl]carbonyl}-3-azabicyclo[4.1.0]hept-4-yl)methyl]-2-pyrazinamine hydrochloride

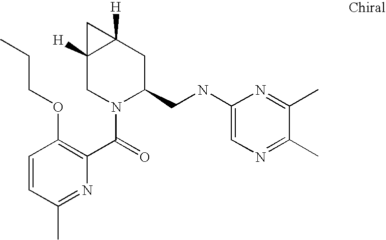

### Example 59

### N-[((1R,4S,6R)-3-{[6-methyl-3-(4-methyl-1,3-oxazol-2-yl)-2-pyridinyl]carbonyl}-3-azabicyclo[4.1.0]hept-4-yl)methyl]-5-(trifluoromethyl)-2-pyrimidinamine hydrochloride

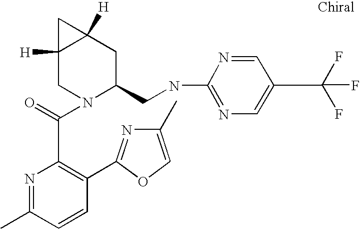

### Example 60

### Determination of antagonist affinity at human Orexin-1 and 2 receptors using FLIPR

### Cell Culture

Adherent Chinese Hamster Ovary (CHO) cells, stably expressing the recombinant human Orexin-1 or human Orexin-2 receptors or Rat Basophilic Leukaemia Cells (RBL) stably expressing recombinant rat Orexin-1 or rat Orexin-2 receptors were maintained in culture in Alpha Minimum Essential Medium (Gibco/Invitrogen, cat. no.; 22571-020), supplemented with 10% decomplemented foetal bovine serum (Life Technologies, cat. no. 10106-078) and 400 μg/mL Geneticin G418 (Calbiochem, cat. no. 345810). Cells were grown as monolayers under 95%:5% air: CO₂ at 37° C.

The sequences of the human orexin 1, human orexin 2, rat orexin 1 and rat orexin 2 receptors used in this example were as published in Sakurai, T. et al (1998) Cell, 92 pp 573 to 585. Some examples were tested against the human orexin 1 receptor as published in Sakurai et al supra with the exception that the amino acid residue at position 280 was alanine and not glycine.

### Measurement of [Ca²⁺]ᵢ Using the FLIPR™

Cells were seeded into black clear-bottom 384-well plates (density of 20,000 cells per well) in culture medium as described above and maintained overnight (95%:5% air:CO₂ at 37° C.). On the day of the experiment, culture medium were discarded and the cells washed three times with standard buffer (NaCl, 145 mM; KCl, 5 mM; HEPES, 20 mM; Glucose, 5.5 mM; MgCl₂, 1 mM; CaCl₂, 2 mM) added with Probenecid 2.5 mM. The plates were then incubated at 37° C. for 60 minutes in the dark with 2 μM FLUO-4AM dye to allow cell uptake of the FLUO-4AM, which is subsequently converted by intracellular esterases to FLUO-4, which is unable to leave the cells. After incubation, cells were washed three times with standard buffer to remove extracellular dye and 30 μL of buffer were left in each well after washing.

Compounds of the invention were tested in a final assay concentration range from 1.66×10⁻⁵M to 1.58×10⁻¹¹M. Compounds of the invention were dissolved in dimethylsulfoxide (DMSO) at a stock concentration of 10 mM. These stock solutions were serially diluted with DMSO and 1 μL of each dilution was transferred to a 384 well compound plate. Immediately before introducing compound to the cells, buffer solution (50 μl/well) was added to this plate. To allow agonist stimulation of the cells, a stock plate containing a solution of human orexin A (hOrexin A) was diluted with buffer to final concentration just before use. This final concentration of hOrexin A was equivalent to the calculated EC80 for hOrexinA agonist potency in this test system. This value was obtained by testing hOrexinA in concentration response curve (at least 16 replicates) the same day of the experiment.

The loaded cells were then incubated for 10 min at 37° C. with test compound. The plates were then placed into a FLIPR™ (Molecular Devices, UK) to monitor cell fluorescence (λₑₓ=488 nm, λ_{EM}=540 nm) (Sullivan E, Tucker E M, Dale I L. Measurement of [Ca²⁺]ᵢ using the fluometric imaging plate reader (FLIPR). In: Lambert DG (ed.), Calcium Signaling Protocols. New Jersey: Humana Press, 1999, 125-136). A baseline fluorescence reading was taken over a 5 to 10 second period, and then 10 μL of EC80 hOrexinA solution was added. The fluorescence was then read over a 45 minute period.

### Data Analysis

Functional responses using FLIPR were measured as peak fluorescence intensity minus basal fluorescence and expressed as a percentage of a non-inhibited Orexin-A-induced response on the same plate. Iterative curve-fitting and parameter estimations were carried out using a four parameter logistic model and Microsoft Excel (Bowen W P, Jerman J C. Nonlinear regression using spreadsheets. Trends Pharmacol. Sci. 1995; 16: 413-417). Antagonist affinity values (IC₅₀) were converted to functional pKᵢ values using a modified Cheng-Prusoff correction (Cheng Y C, Prusoff W H. Relationship between the inhibition constant (Kᵢ) and the concentration of inhibitor which causes 50 percent inhibition (IC₅₀) of an enzymatic reaction. Biochem. Pharmacol. 1973, 22: 3099-3108).

$$fpKi = - log \frac{( {IC}_{50} )}{{( 2 + {( \frac{[ agonist ]}{( {EC}_{50} )} )}^{n} )}^{1 / n} - 1}$$

Where [agonist] is the agonist concentration, EC₅₀ is the concentration of agonist giving 50% activity derived from the agonist dose response curve and n=slope of the dose response curve. When n=1 the equation collapses to the more familiar Cheng-Prusoff equation.

The compounds of examples 1 to 47, 49 and 50 were tested according to the method of example 60. All compounds gave fpKi values from 7.9 to 10.1 at the human cloned orexin-1 receptor (as published in Sakuri et al supra or having the amino acid residue alanine at position 280 and not glycine) and from 5.8 to 9.4 at the human cloned orexin-2 receptor.

Compounds of the following examples tested according to this example gave fpKi values as follows:

| Example | Orexin 1 receptor | Orexin 2 receptor |
|---|---|---|
| 51 | 9.2 | 7.8 |
| 52 | 9.2 | 9.4 |
| 53 | 6.0 | <5.48 |
| 54 | 9.3 | 8.4 |
| 55 | 9.1 | 7.0 |
| 56 | 9.5 | 6.4 |
| 57 | 9.5 | 7.2 |
| 58 | 9.0 | 8.9 |
| 59 | 6.0 | 5.8 |

1. A compound which is N-[((1R,4S,6R)-3-{[6-methyl-3-(2-pyrimidinyl)-2-pyridinyl]carbonyl}-3-azabicyclo[4.1.0]hept-4-yl)methyl]-5-(trifluoromethyl)-2-pyridinamine, having the formula: 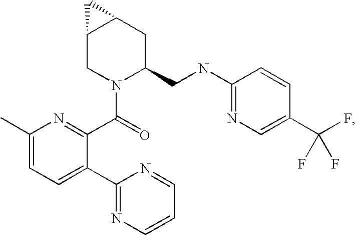
or a pharmaceutically acceptable salt thereof.

2. A method for the treatment of a disease or disorder where an antagonist of a human orexin receptor is required, in a human in need thereof, comprising administering to said human an effective amount of the compound or salt as defined in claim 1, where the disease or disorder is a sleep disorder, a depression or mood disorder, an anxiety disorder, a substance-related disorder or a feeding disorder.

3. A method according to claim 2, where the disease or disorder is a sleep disorder.

4. A method according to claim 2, where the disease or disorder is insomnia.

5. A method according to claim 3, where the sleep disorder is selected from the group consisting of Primary Insomnia; Breathing-Related Sleep Disorders; Circadian Rhythm Sleep Disorder; Dyssomnia Not Otherwise Specified; Nightmare Disorder; Sleep Terror Disorder; Sleepwalking Disorder; Parasomnia Not Otherwise Specified; Insomnia Related to Another Mental Disorder; insomnia associated with a disease selected from a neurological disorder, neuropathic pain, restless leg syndrome, heart disease and lung disease; Insomnia Type Substance-Induced Sleep Disorder; Parasomnia Type Substance-Induced Sleep Disorder; Mixed Type Substance-Induced Sleep Disorder; Sleep Apnea; and Jet-Lag Syndrome.

6. A pharmaceutical composition comprising the compound or salt as defined in claim 1, and one or more pharmaceutically acceptable carriers.
